(19) United States
(12) Patent Application Publication
(10) Pub. No.: US 2021/0276967 A1
(43) Pub. Date: Sep. 9, 2021

(54) INHIBITORS OF HEPATITIS B VIRUS

(71) Applicants: OSPEDALE SAN RAFFAELE S.R.L., Milano (MI) (IT); IRBM S.P.A., Pomezia (RM) (IT); PROMIDIS S.R.L., Milano (MI) (IT); ISTITUTO NAZIONALE DI GENETICA MOLECOLARE - INGM, Milano (MI) (IT)

(72) Inventors: Raffaele DE FRANCESCO, Milano (MI) (IT); Adolfo PRANDI, Milano (MI) (IT); Pietro RANDAZZO, Milano (MI) (IT); Lorena DONNICI, Milano (MI) (IT); Luca GUIDOTTI, Milano (MI) (IT); Matteo IANNACONE, Milano (MI) (IT); Romano DI FABIO, Pomezia (RM) (IT); Vincenzo SUMMA, Pomezia (RM) (IT); Leda Ivanova BENCHEVA, Milano (MI) (IT); Marilenia DE MATTEO, Milano (MI) (IT); Luca FERRANTE, Milano (MI) (IT)

(73) Assignees: OSPEDALE SAN RAFFAELE S.R.L., Milano (MI) (IT); IRBM S.P.A., Pomezia (RM) (IT); PROMIDIS S.R.L., Milano (MI) (IT); ISTITUTO NAZIONALE DI GENETICA MOLECOLARE - INGM, Milano (MI) (IT)

(21) Appl. No.: 17/261,223

(22) PCT Filed: Jul. 19, 2019
(86) PCT No.: PCT/EP2019/069550

§ 371 Date: Jan. 19, 2021

(30) Foreign Application Priority Data
Jul. 19, 2018 (EP) 18184456.4

(51) Int. Cl. C07D 305/08; A61K 45/06; C07D 207/48; C07D 213/42; C07D 205/04; C07D 213/73; C07D 211/96; C07C 311/16

(52) U.S. Cl. CPC C07D 305/08; A61K 45/06; C07D 207/48; C07C 311/16; C07D 205/04; C07D 213/73; C07D 211/96; C07D 213/42

(57) ABSTRACT

The present invention relates to compounds that are inhibitors of hepatitis B virus (HBV). Compounds of this invention are useful alone or in combination with other agents for treating, ameliorating, preventing or curing HBV infection and related conditions. The present invention also relates to pharmaceutical compositions containing said compounds.

### FIELD OF THE INVENTION

The present invention relates to compounds that are inhibitors of hepatitis B virus (HBV). Compounds of this invention are useful alone or in combination with other agents for treating, ameliorating, preventing or curing HBV infection and related conditions. The present invention also relates to pharmaceutical compositions containing said compounds.

### BACKGROUND OF THE INVENTION

The Hepatitis B virus (HBV) is an enveloped, partially double-stranded DNA (dsDNA) virus of the hepadnaviridae family that is spread by contact with infected blood and body fluids and causes acute and chronic necroinflammatory liver diseases of varying severity (Guidotti L G, Chisari F V. Annu Rev Pathol. 2006; 1:23-61). The HBV lipid envelope contains 3 in-frame viral envelope proteins (large, middle and small), each of which possesses the hepatitis B virus surface antigen (HBsAg) determinant (Seeger C, Mason W S.Virology. 2015 May; 479-480:672-86). This envelope encloses a protein shell, or capsid, that is composed of 240 monomers of the core protein and each monomer possesses the hepatitis B virus core antigen (HBcAg or Cp) determinant. The capsid in turn encloses a partially double-stranded, relaxed circular DNA (rcDNA) form of the viral genome as well as a molecule of the viral polymerase. Upon entry into susceptible cells (i.e. the hepatocytes) via the interaction of the large envelope protein with specific receptors on the hepatocellular membrane, the capsid is released into the cytoplasm and transported at the nuclear membrane. The rcDNA is then released into the nucleus and repaired by cellular polymerases into an episomal “minichromosome”, termed covalently closed circular DNA (cccDNA), which represents the viral transcriptional template. The minus strand of the viral DNA encodes 3.5, 2.4, 2.1 and 0.7 kb mRNA species that are translated into structural (envelope and core) and nonstructural (polymerase, precore and X) proteins of the virus. Following transport into the cytoplasm, one of the 3.5 kb RNAs (termed pregenomic RNA) is selectively packaged into a nascent capsid by interacting with the core and polymerase proteins that have been translated from their respective mRNAs. Within these capsids, the viral polymerase reverse transcribes the pregenomic RNA into a single minus (−) strand DNA molecule that serves as template for the viral polymerase-mediated DNA plus (+) strand synthesis and the cohesive structure of the linear DNA intermediates converts them into a relaxed circular double stranded molecule. A fraction of these HBV DNA-containing “mature” capsids are transported back to the nucleus where second strand synthesis is completed and the ends of both strands are ligated, leading to amplification of the pool of cccDNA. Another fraction of the capsids binds to viral envelope proteins that have been independently translated and translocated to membranes of endoplasmic reticulum (ER)-like structures. Following binding, the enveloped capsids bud into the lumen of the ER and exit the cell as infectious virions to initiate new cycles of infection.

Thus, the HBV core protein and the related capsids are essential components and regulators of the HBV life cycle. The full-length core protein Cp183, or its N-terminal domain Cp149, predominantly assembles into a T=4 icosahedral capsids. Due to its critical roles in capsid assembly, pregenomic RNA packaging, and cccDNA maintenance, it is not surprising that the HBV core protein and the related capsids have been widely recognized as attractive antiviral targets (Durantel D, Zoulim F; J Hepatol. 2016 April; 64(1 Suppl):S117-S131).

According to World Health Organization (WHO) statistics, HBV infection is one of the major medical scourges of our time. As a sexually transmitted disease that is also transferred by intravenous drug abuse and from mother to infant at birth, over one third of the world's population has been infected by HBV at some point in their lives (Burns G S, Thompson A J; Cold Spring Harb Perspect Med. Oct. 30, 2014; 4(12)). While most of these people have successfully cleared the virus, more than 250 million people remain persistently infected and almost 900,000 of these individuals die annually from the complications of chronic infection (i.e. cirrhosis and/or hepatocellular carcinoma). HBV infection is highly endemic in sub-Saharan Africa, the Pacific, and particularly Asia. Regions with high rates of chronic HBV infection also include the Middle East, the Indian subcontinent, areas of South and Central America, and the southern parts of Eastern and Central Europe. In recent years the number of chronic carriers has increased steadily in the western world as well, mostly because of the influx of immigrants from endemic areas. Additionally, HBV acts as a helper virus to hepatitis delta virus (HDV) and it should be noted that the more than 15 million people co-infected with HBV and HDV have an increased risk of rapid progression to cirrhosis and hepatic decompensation (Hughes, S. A. et al. Lancet 2011, 378, 73-85).

Well-tolerated vaccines that elicit neutralizing antibodies to HBsAg efficiently prevent de novo HBV infection, but have no therapeutic potential for the millions of people that are already persistently infected (Zoulim, Durantel D; Cold Spring Harb Perspect Med. Apr. 1, 2015; 5(4)). Therapy for these individuals mainly relies on direct acting antiviral (DAA) drugs (e.g. tenofovir, lamivudine, adefovir, entecavir or telbivudine) that suppress virus production but do not eradicate HBV from the liver, requiring lifelong treatment. Cohorts of patients still receive a therapy based on pegylated interferon-α (PEG-IFN-α), which has the advantages of limited treatment duration and higher rates of HBsAg seroconversion but the relevant disadvantage of greater adverse effects. As such, the number of patients receiving PEG-IFN-α is progressively decreasing.

Different chemical classes of inhibitors targeting the encapsidation process of HBV (also termed capsid assembly modulators or CAMs) are under development, and they include heteroaryldihydropyrimidines (HAPs) and sulfamoylbenzamides (SBAs). For instance, Novira Therapeutics recently utilized a humanized mouse model of HBV infection to show that a combination of CAM and PEG-IFN-α has higher antiviral activity than that previously observed with DAAs. NVR3-778, the first member of this class of CAM, in Phase 1b proof-of-concept clinical studies showed both significant reduction in HBV DNA and serum HBV RNA. This compound was recently discontinued. The compound JNJ-56136379 (or JNJ-379), developed by Janssen, has recently demonstrated potent antiviral activity and is now entering into Phase 2 clinical trial.

WO2013/006394, published on Jan. 10, 2013, relates to a subclass of sulfamoyl-arylamides having general formula A, useful for the treatment of Hepatitis B virus (HBV) infection:

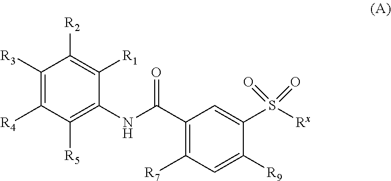

WO2013/096744, published on Jun. 26, 2013, relates to sulfamoyl-arylamides of formula B active against HBV:

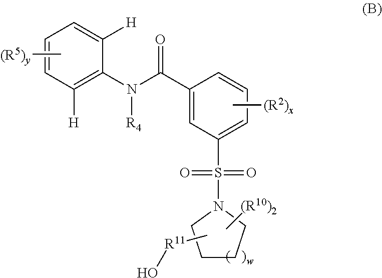

WO2014/106019, published on Jul. 3, 2014, relates to compounds of formula C, useful as nucleocapsid assembly inhibitors for the treatment of viruses, especially but not exclusively, including pregenomic RNA encapsidation inhibitors of HBV for the treatment of Hepatitis B virus (HBV) infection and related conditions:

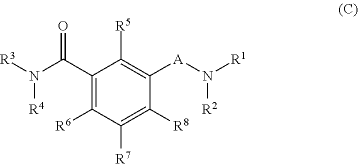

WO2014/165128, published on Oct. 9, 2014, WO2015/109130 published on Jul. 23, 2015, US2015274652, published on Oct. 1, 2015, all relate to sulfamoyl-arylamides compounds active against HBV.

WO2015/120178, published on Aug. 13, 2015, relates to sulfamoyl-arylamides compounds used in combination therapy with peginterferon alfa-2a, or another interferon analog for the treatment of HBV infection.

WO2016/089990, published on Jun. 9, 2016, relates to sulfide alkyl and pyridyl reverse sulphonamide compounds for HBV treatment.

US2016185748, published on Jun. 30, 2016, relates to pyridyl reverse sulfonamides for HBV treatment.

US2016151375, published on Jun. 2, 2016 relates to sulfide alkyl compounds for HBV treatment.

WO2017/001655A1 published on Jan. 5, 2017, relates to cyclized sulfamoylarylamide derivatives.

JP49040221 (also published as GB 1,313,217) describes compound 2-amino-N-(4-chloro-2-methylphenyl)-5-sulfamoylbenzamide (CAS no. 55455-09-9).

WO2010/123139 describes compound N-(2-methoxyphenyl)-2-(methylamino)-5-(piperidin-1-ylsulfonyl)benzamide (CAS no. 1253220-93-7).

Amongst the problems which HBV direct antivirals may encounter are toxicity, mutagenicity, lack of selectivity, poor efficacy, poor bioavailability, low solubility, and/or off-target activity and to date no compounds in any of the structural classes identified above have been approved as a drug for the treatment of HBV patients.

There is a need for additional HBV inhibitors that may overcome at least one of these disadvantages or that have additional advantages such as increased potency, increased bioavailability or an increased safety window.

The present invention provides small molecule drugs obtained through chemical modification of the known sulfamoyl arylamides derivatives. From the structural point of view, the distinguishing feature characterizing the sulfamoyl amides of the invention is the presence of an amino group ortho or para to the sulfamoyl group. This substitution pattern results in potent HBV inhibitors with improved pharmacokinetic properties, good kinetic solubility, stability in mouse and human hepatocytes, low in vivo clearance and positive liver-to-plasma concentration. Given the liver's key role in metabolic regulation and the fact that it is the principal tissue affected by hepatitis B disease, designing HBV inhibitors with hepatoselective distribution profiles is an important strategy in developing safe drug candidates (Tu M. et al., Current Topics in Medicinal Chemistry, 2013, 13, 857-866).

### DESCRIPTION OF THE INVENTION

The compounds of this invention are inhibitors of hepatitis B virus (HBV).

It is therefore an object of the present invention a compound of general formula (I):

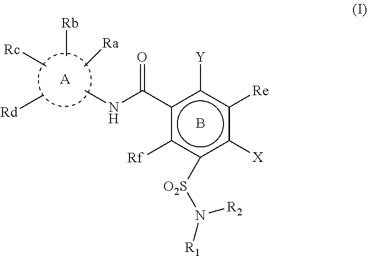

wherein:

A is a 6-membered aromatic or heteroaromatic ring;

B is a 6-membered aryl optionally containing one or more N atoms;

X is H or NR₃R₄;

Y is selected from the group consisting of hydrogen, halogen, C₁₋₆alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;

with the proviso that, when X is H, Y is selected form the group consisting of NH₂, NH(C₁₋₆alkyl), N(CH₃)₂, NHC(O)CH₃;

R₁ and R₂ are each independently selected from H, linear or branched C₁₋₆alkyl, saturated or partially unsaturated C₃₋₇cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀heterocycloalkyl;

or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl and (CH₂)ₙR₅;

each occurrence of n is independently 0, 1, 2, 3 or 4;

R₃ and R₄ are each independently H, or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;

R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;

Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O) CH₃, OH and CN; or is absent;

Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rd is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Re is selected from the group consisting of hydrogen, halogen, C₁₋₃alkyl; or is absent;

Rf is hydrogen, halogen, C₁₋₃alkyl; or is absent;

provided that the compound is not 2-amino-N-(4-chloro-2-methylphenyl)-5-sulfamoylbenzamide or N-(2-methoxyphenyl)-2-(methylamino)-5-(piperidin-1-ylsulfonyl)benzamide; and pharmaceutically acceptable salts, tautomers, isomers, stereoisomers thereof.

Preferably, A is phenyl. Preferably, B is phenyl. Preferably, A and B are both phenyl.

Preferably, X is NR₃R₄, wherein more preferably R₃ and R₄ are both H.

Preferably, Y is selected from the group consisting of: hydrogen, halogen (in particular Cl or Br), C₁₋₆alkyl (in particular methyl) and NH₂. In another preferred embodiment, X is hydrogen and Y is NH₂.

Preferably, R₁ and R₂ are each independently selected from: hydrogen, linear or branched C₁₋₆alkyl optionally substituted with halogen, saturated C₃₋₆cycloalkyl optionally substituted with OH or with NH(C═O)OC₁₋₆alkyl and C₃₋₆heterocycloalkyl optionally substituted with NH(C═O)OC₁₋₆alkyl. In a preferred embodiment, R₁ and R₂ taken together form with the N atom to which they are attached a saturated 4-6 membered heterocyclic ring optionally substituted with OH or with CH₂OH. More preferably, R₁ is hydrogen, methyl, or is selected from the group consisting of:

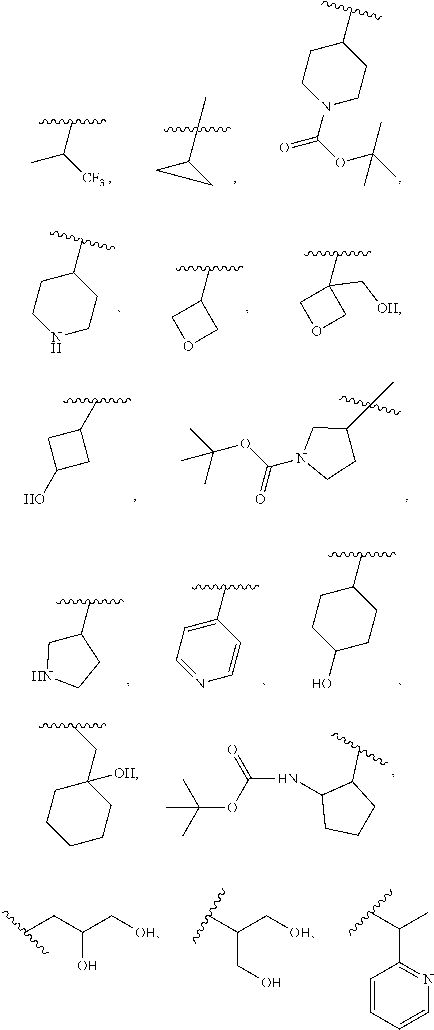

and

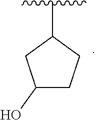 [SMILES: *C1CCC(O)C1]

Also preferably, R₂ is H or methyl.

Preferably, R₃ and R₄ are both H. Preferably, R₅ is OH. Preferably, Ra is H. Preferably Rb and Rd are each independently selected from the group consisting of: hydrogen, F, CF₃, CN, CHF₂, Cl and methyl. Preferably, Rc is F. Preferably, Re is hydrogen or C₁₋₃alkyl, in particular methyl. Preferably, Rf is hydrogen.

In a preferred embodiment, the compound of the invention has general formula (Ia):

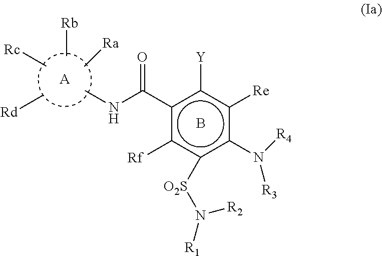

wherein:

A is a 6-membered aromatic or heteroaromatic ring;

B is a 6-membered aryl optionally containing one or more N atoms;

Y is selected from the group consisting of hydrogen, halogen, C₁₋₃alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;

R₁ and R₂ are each independently selected from H, linear or branched C₁₋₆alkyl, saturated or partially unsaturated C₃₋₇cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀heterocycloalkyl; or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl, (CH₂)ₙR₅;

each occurrence of n is independently 0, 1, 2, 3 or 4;

R₃ and R₄ are each independently H or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;

R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;

Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rd is selected from the group consisting hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Re is selected from the group consisting of hydrogen, halogen and C₁₋₃alkyl; or is absent;

Rf is hydrogen, halogen and C₁₋₃alkyl; or is absent;

and pharmaceutically acceptable salts, tautomers, isomers, stereoisomers thereof.

In a further preferred embodiment, the invention relates to a compound of formula (Ia) wherein:

A is a 6-membered aromatic or heteroaromatic ring;

B is a 6-membered aryl optionally containing one or more N atoms;

Y is selected from the group consisting of hydrogen, halogen, C₁₋₃alkyl, NH₂, NH(C₁₋₆alkyl), N(CH₃)₂, NHC(O)CH₃, OH and CN or is absent;

R₁ is H, linear or branched C₁₋₆alkyl, cyclopropyl, cyclobutyl, cyclopentyl, cyclohexyl, piperidinyl, pirrolidinyl, oxetanyl, tetrahydrofuranyl, pyridinyl, said C₁₋₆alkyl, cyclopropyl, cyclobutyl, cyclopentyl, cyclohexyl, piperidinyl, pirrolidinyl, oxetanyl, tetrahydrofuranyl or pyridinyl being optionally substituted with one or more substituents selected from OH, halogen, NH₂, NH(C═O)OC₁₋₆alkyl, NH(C₁₋₆alkyl), C₁₋₆hydroxyalkyl, 5- or 6-membered heteroaryl, C(═O)C₁₋₆alkyl, C(═O)OC₁₋₆alkyl, OC₁₋₆alkyl;

R₂ is H or methyl;

or R₁ and R₂ taken together form with the N atom to which they are attached a heterocyclic ring selected from piperidine, pirrolidine, morpholine, thiomorpholine and piperazine, said ring being optionally substituted with one or more substituents selected from halogen, C₁₋₃alkyl, OH and CH₂R₅;

R₃ and R₄ are each independently H or C₁₋₃alkyl; in particular hydrogen or methyl;

R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;

Ra is selected from the group consisting of hydrogen, halogen, C₁₋₃alkyl, haloC₁₋₃alkyl and CN; or is absent;

Rb is selected from the group consisting of hydrogen, halogen, C₁₋₃alkyl, haloC₁₋₃alkyl and CN; or is absent;

Rc is selected from the group consisting of hydrogen, halogen, C₁₋₃alkyl, haloC₁₋₃alkyl and CN; or is absent;

Rd is selected from the group consisting of hydrogen, halogen, C₁₋₃alkyl, haloC₁₋₃alkyl and CN; or is absent;

Re is selected from the group consisting of hydrogen, halogen, C₁₋₃alkyl, or is absent;

Rf is hydrogen or is absent;

and pharmaceutically acceptable salts, tautomers, isomers, stereoisomers thereof.

In a preferred embodiment, the compounds of the invention have formula (Ia), wherein:

A is a 6-membered aromatic or heteroaromatic ring;

B is a 6-membered aryl optionally containing one or more N atoms;

Y is selected from the group consisting of hydrogen, halogen, C₁₋₃alkyl, or is absent;

R₁ is hydrogen, methyl, or is selected from the group consisting of:

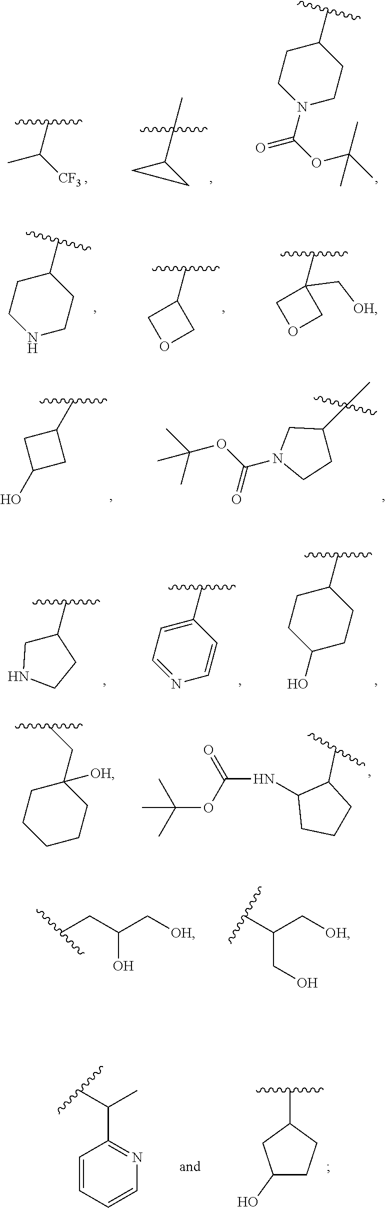

R₂ is H or methyl;

or R₁ and R₂ taken together form with the N atom to which they are attached a heterocyclic ring selected from the group consisting of:

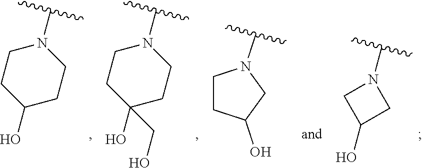

R₃ and R₄ are each independently H or C₁₋₃alkyl; in particular hydrogen or methyl;

Ra is selected from the group consisting of hydrogen, halogen, C₁₋₃alkyl, haloC₁₋₃alkyl and CN; or is absent;

Rb is selected from the group consisting of hydrogen, halogen, C₁₋₃alkyl, haloC₁₋₃alkyl and CN; or is absent;

Rc is selected from the group consisting of hydrogen, halogen, C₁₋₃alkyl, haloC₁₋₃alkyl and CN; or is absent;

Rd is selected from the group consisting of hydrogen, halogen, C₁₋₃alkyl, haloC₁₋₃alkyl and CN; or is absent;

Re is selected from the group consisting of hydrogen, halogen, C₁₋₃alkyl, or is absent;

Rf is hydrogen; or is absent;

and pharmaceutically acceptable salts, tautomers, isomers, stereoisomers thereof.

Preferably, A is phenyl or pyridyl. Preferably, B is phenyl or pyridyl. Preferably, A is phenyl and B is phenyl.

Preferably, at least one of Ra, Rb, Rc and Rd is F and the other(s) is/are hydrogen.

All the definitions of substituents, such as for example “alkyl”, “alkoxy”, “aryl”, “heteroaryl” and so on, are reported herein below and apply to formula (I) and formula (Ia).

In a further embodiment, the invention relates to a compound of formula (Ia) wherein R₁, R₂, R₃, R₄, R₅, Re, Rf and Y are as defined above and A is phenyl or pyridyl, B is phenyl or pyridyl; and

Ra is selected from the group consisting of hydrogen, halogen, C₁₋₃alkyl, haloC₁₋₃alkyl and CN;

Rb is selected from the group consisting of hydrogen, halogen, C₁₋₃alkyl, haloC₁₋₃alkyl and CN;

Rc is selected from the group consisting of hydrogen, halogen, C₁₋₃alkyl, haloC₁₋₃alkyl and CN;

Rd is selected from the group consisting of hydrogen, halogen, C₁₋₃alkyl, haloC₁₋₃alkyl and CN;

and pharmaceutically acceptable salts, tautomers, isomers, stereoisomers thereof.

In a further embodiment, the invention relates to a compound of formula (Ia) wherein R₁, R₂, R₃, R₄, R₅, Re, Rf and Y are as defined above, and A is phenyl, B is phenyl; and

Ra is selected from the group consisting of hydrogen, halogen, C₁₋₃alkyl, haloC₁₋₃alkyl and CN;

Rb is selected from the group consisting of hydrogen, halogen, C₁₋₃alkyl, haloC₁₋₃alkyl and CN;

Rc is selected from the group consisting of hydrogen, halogen, C₁₋₃alkyl, haloC₁₋₃alkyl and CN;

Rd is selected from the group consisting of hydrogen, halogen, C₁₋₃alkyl, haloC₁₋₃alkyl and CN; and pharmaceutically acceptable salts, tautomers, isomers, stereoisomers thereof.

In a further embodiment the invention relates to a compound of formula (Ia) wherein R₁, R₂, R₃, R₄, R₅, Re, Rf and Y are as defined above and A is phenyl; B is phenyl; at least one of Ra, Rb, Rc and Rd are F and the other(s) is/are hydrogen; and pharmaceutically acceptable salts, tautomers, isomers, stereoisomers thereof.

In a further embodiment the invention relates to a compound of formula (Ia) wherein R₁, R₂, R₃, R₄, R₅, Re, Rf and Y are as defined above and A is phenyl; B is phenyl; at least two of Ra, Rb, Rc and Rd are F and the other(s) is/are hydrogen; and pharmaceutically acceptable salts, tautomers, isomers, stereoisomers thereof.

In a further embodiment, the invention relates to a compound having formula (Ia) wherein R₁, R₂, R₃, R₄, R₅, Re, Rf and Y are as defined above and A is phenyl; B is phenyl; at least three of Ra, Rb, Rc and Rd are F and the other(s) is/are hydrogen; and pharmaceutically acceptable salts, tautomers, isomers, stereoisomers thereof.

In a further embodiment, the invention relates to a compound of formula (Ia) wherein R₁, R₂, R₃, R₄, R₅, Re, Rf and Y are as defined above and A is phenyl, B is phenyl; Ra, Rc and Rd are each independently hydrogen or F; Rb is selected from the group consisting of methyl, Cl, CF₃, CHF₂ and CN; and pharmaceutically acceptable salts, tautomers, isomers, stereoisomers thereof.

In a further embodiment, the invention relates to a compound wherein R₁, R₂, R₄, R₅, Re, Rf and Y are as defined above and A is phenyl, B is phenyl; R₃ are each hydrogen; and

Ra is selected from the group consisting of hydrogen, halogen, C₁₋₃alkyl, haloC₁₋₃alkyl and CN;

Rb is selected from the group consisting of hydrogen, halogen, C₁₋₃alkyl, haloC₁₋₃alkyl and CN;

Rc is selected from the group consisting of hydrogen, halogen, C₁₋₃alkyl, haloC₁₋₃alkyl and CN;

Rd is selected from the group consisting of hydrogen, halogen, C₁₋₃alkyl, haloC₁₋₃alkyl and CN;

and pharmaceutically acceptable salts, tautomers, isomers, stereoisomers thereof.

In particular, preferred compounds are selected from the following list:
4-amino-3-(N-methylsulfamoyl)-N-(3,4,5-trifluorophenyl)benzamide;
4-amino-3-sulfamoyl-N-(3,4,5-trifluorophenyl)benzamide;
4-amino-N-(3,4-difluorophenyl)-3-sulfamoylbenzamide;
4-amino-2-chloro-5-sulfamoyl-N-(3,4,5-trifluorophenyl)benzamide;
4-amino-2-bromo-5-sulfamoyl-N-(3,4,5-trifluorophenyl)benzamide;
4-amino-N-(4-fluoro-3-(trifluoromethyl)phenyl)-3-sulfamoylbenzamide;
4-amino-N-(3-cyano-4-fluorophenyl)-3-sulfamoylbenzamide;
4-amino-N-(3-(difluoromethyl)-4-fluorophenyl)-3-sulfamoylbenzamide;
4-amino-N-(3-chloro-4-fluorophenyl)-3-sulfamoylbenzamide;
4-amino-N-(4-fluoro-3-methylphenyl)-3-sulfamoylbenzamide;
4-amino-2-methyl-5-sulfamoyl-N-(3,4,5-trifluorophenyl)benzamide;
(R)-4-amino-N-(3,4,5-trifluorophenyl)-3-(N-(1,1,1-trifluoropropan-2-yl)sulfamoyl)benzamide;
(S)-4-amino-N-(3,4,5-trifluorophenyl)-3-(N-(1,1,1-trifluoropropan-2-yl)sulfamoyl)benzamide;
4-amino-3-(N-cyclopropylsulfamoyl)-N-(3,4,5-trifluorophenyl)benzamide;
trans-4-amino-3-(N-(4-hydroxycyclohexyl)sulfamoyl)-N-(3,4,5-trifluorophenyl)benzamide;
cis-4-amino-3-(N-(4-hydroxycyclohexyl)sulfamoyl)-N-(3,4,5-trifluorophenyl)benzamide;
trans-4-amino-5-(N-(4-hydroxycyclohexyl)sulfamoyl)-2-methyl-N-(3,4,5-trifluorophenyl)benzamide;
cis-4-amino-3-(N-3-hydroxycyclobutyl)sulfamoyl)-N-(3,4,5-trifluorophenyl)benzamide;
trans-4-amino-3-(N-3-hydroxycyclobutyl)sulfamoyl)-N-(3,4,5-trifluorophenyl)benzamide;
4-amino-3-(N-((1R,3R)-3-hydroxycyclopentyl)sulfamoyl)-N-(3,4,5-trifluorophenyl)benzamide;
4-amino-3-((4-hydroxypiperidin-1-yl)sulfonyl)-N-(3,4,5-trifluorophenyl)benzamide;
4-amino-3-(N-(oxetan-3-yl)sulfamoyl)-N-(3,4,5-trifluorophenyl)benzamide;
tert-butyl(S)-3-((2-amino-5-((3,4,5-trifluorophenyl)carbamoyl)phenyl)sulfonamido) pyrrolidine-1-carboxylate;
4-amino-3-methyl-5-sulfamoyl-N-(3,4,5-trifluorophenyl)benzamide;
4-amino-3-(N-(3-(hydroxymethyl)oxetan-3-yl)sulfamoyl)-N-(3,4,5-trifluorophenyl)benzamide;
4-amino-3-(N-((1-hydroxycyclohexyl)methyl)sulfamoyl)-N-(3,4,5-trifluorophenyl)benzamide;
4-amino-N-(4-fluoro-3-methylphenyl)-2-methyl-5-sulfamoylbenzamide;
4-amino-5-(N-((1R,4R)-4-hydroxycyclohexyl)sulfamoyl)-2-methyl-N-(3,4,5-trifluorophenyl) benzamide;
trans-4-amino-N-(3-chloro-4-fluorophenyl)-3-(N-(4-hydroxycyclohexyl)sulfamoyl)benzamide;
4-amino-N-(3-(difluoromethyl)-4-fluorophenyl)-3-(N-((1r1R,4r4R)-4-hydroxycyclohexyl) sulfamoyl)benzamide;
trans-4-amino-N-(3-(difluoromethyl)-4-fluorophenyl)-3-(N-(4-hydroxycyclohexyl)sulfamoyl) benzamide;
4-amino-3-(N-((1S,3R)-3-hydroxycyclopentyl)sulfamoyl)-N-(3,4,5-trifluorophenyl)benzamide;
4-amino-3-(N-((1R,3S)-3-hydroxycyclopentyl)sulfamoyl)-N-(3,4,5-trifluorophenyl)benzamide;
4-amino-3-((4-hydroxy-4-(hydroxymethyl)piperidin-1-yl)sulfonyl)-N-(3,4,5-trifluorophenyl)benzamide;
tert-butyl((1R,2S)-2-((2-amino-5-((3,4,5-trifluorophenyl)carbamoyl)phenyl)sulfonamido) cyclopentyl)carbamate;
tert-butyl((1S,2R)-2-((2-amino-5-((3,4,5-trifluorophenyl)carbamoyl)phenyl)sulfonamido) cyclopentyl)carbamate;
4-amino-3-((3-hydroxypyrrolidin-1-yl)sulfonyl)-N-(3,4,5-trifluorophenyl)benzamide;
4-amino-N-(3-chloro-4-fluorophenyl)-3-((4-hydroxypiperidin-1-yl)sulfonyl)benzamide;
4-amino-N-(3-chloro-4-fluorophenyl)-3-((3-hydroxyazetidin-1-yl)sulfonyl)benzamide;
4-amino-3-(N-(2,3-dihydroxypropyl)sulfamoyl)-N-(3,4,5-trifluorophenyl)benzamide;
trans-4-amino-3-(N-(3-hydroxycyclopentyl)sulfamoyl)-N-(3,4,5-trifluorophenyl)benzamide;
trans-2-amino-5-(N-(4-hydroxycyclohexyl)sulfamoyl)-N-(3,4,5-trifluorophenyl)benzamide;
2-amino-5-((4-hydroxypiperidin-1-yl)sulfonyl)-N-(3,4,5-trifluorophenyl)benzamide;
(R)-4-amino-2-methyl-N-(3,4,5-trifluorophenyl)-5-(N-(1,1,1-trifluoropropan-2-yl)sulfamoyl) benzamide;
(S)-4-amino-2-methyl-N-(3,4,5-trifluorophenyl)-5-(N-(1,1,1-trifluoropropan-2-yl)sulfamoyl) benzamide;
4-amino-N-(3-chloro-4,5-difluorophenyl)-2-methyl-5-sulfamoylbenzamide;
4-amino-N-(4-fluoro-3-(trifluoromethyl)phenyl)-2-methyl-5-sulfamoylbenzamide;
4-amino-N-(3-(difluoromethyl)-4-fluorophenyl)-2-methyl-5-sulfamoylbenzamide;
4-amino-N-(3-cyano-4-fluorophenyl)-2-methyl-5-sulfamoylbenzamide;
4-amino-N-(3-chloro-4-fluorophenyl)-2-methyl-5-sulfamoylbenzamide;
and pharmaceutically acceptable salts, tautomers, isomers, stereoisomers thereof.
In particular, compounds 4-amino-3-sulfamoyl-N-(3,4,5-trifluorophenyl)benzamide and 4-amino-2-methyl-5-sulfamoyl-N-(3,4,5-trifluorophenyl)benzamide are preferred.

Preferred compounds exhibit an HBV inhibition percentage activity, as defined hereinbelow, greater than 50% at the test concentration (preferably greater than 60%, more preferably greater than 75%) and/or an EC₅₀, as defined hereinbelow, lower than 1 μM. HBV inhibition indicates inhibition of HBV expression and/or replication. The inhibition activity of the compound of the invention can be measured as described hereinafter.

It is an object of the invention a compound as defined above for medical use. Preferably, the compound as defined above is for use in the treatment and/or prevention of an HBV infection and/or a condition related to an HBV infection.

In a preferred embodiment, the compound for use in the treatment and/or prevention of an HBV infection and/or a condition related to an HBV infection has general formula (I):

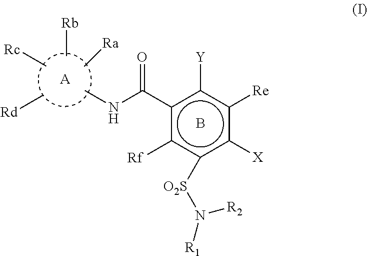

wherein:

A is a 6-membered aromatic or heteroaromatic ring;

B is a 6-membered aryl optionally containing one or more N atoms;

X is H or NR₃R₄;

Y is selected from the group consisting of hydrogen, halogen, C₁₋₆alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;

with the proviso that, when X is H, Y is selected form the group consisting of NH₂, NH(C₁₋₆alkyl), N(CH₃)₂, NHC(O)CH₃;

R₁ and R₂ are each independently selected from H, linear or branched C₁₋₆alkyl, saturated or partially unsaturated C₃₋₇cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀heterocycloalkyl;

or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl and (CH₂)ₙR₅;

each occurrence of n is independently 0, 1, 2, 3 or 4;

R₃ and R₄ are each independently H, or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;

R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;

Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O) CH₃, OH and CN; or is absent;

Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Rd is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;

Re is selected from the group consisting of hydrogen, halogen, C₁₋₃alkyl; or is absent;

Rf is hydrogen, halogen, C₁₋₃alkyl; or is absent;

and pharmaceutically acceptable salts, tautomers, isomers, stereoisomers thereof.

It is a further object of the invention a compound as defined above for use in treating, eradicating, reducing, slowing or inhibiting an HBV infection in an individual in need thereof, and/or in reducing the viral load associated with an HBV infection in an individual in need thereof, and/or in reducing reoccurrence of an HBV infection in an individual in need thereof, and/or in inducing remission of hepatic injury from an HBV infection in an individual in need thereof, and/or in prophylactically treating an HBV infection in an individual afflicted with a latent HBV infection.

Preferably, the compound as defined above is for use in combination with at least one further therapeutic agent. Preferably, said use in combination comprises the administration of at least one therapeutic agent.

It is an object of the invention a pharmaceutical composition comprising the compound as defined above, alone or in combination with at least one further therapeutic agent, and at least one pharmaceutically acceptable excipient.

Preferably, the at least one further therapeutic agent is selected from the group consisting of: a therapeutic vaccine; an RNA interference therapeutic/antisense oligonucleotide; an immunomodulator; a STING agonist; a RIG-I modulator; a NKT modulator; an IL agonist; an interleukin or another immune acting protein; a therapeutic and prophylactic vaccine; an immune checkpoint modulator/inhibitor; an HBV entry inhibitor; a cccDNA modulator; an inhibitor of HBV protein espression; an agent targeting HBV RNA; a capsid assembly inhibitor/modulator; a core or X protein targeting agent; a nucleotide analogue; a nucleoside analogue; an interferon or a modified interferon; an HBV antiviral of distinct or unknown mechanism; a cyclophilin inhibitor; a sAg release inhibitor; an HBV polymerase inhibitor; a dinucleotide; a SMAC inhibitor; a HDV targeting agent; a viral maturation inhibitor; a reverse transcriptase inhibitor and an HBV RNA destabilizer or another small-molecule inhibitor of HBV protein expression; or a combination thereof.

Preferably, the therapeutic vaccine is selected from: HBsAG-HBIG, HB-Vac, ABX203, NASVAC, GS-4774, GX-110 (HB-110E), CVI-HBV-002, RG7944 (INO-1800), TG-1050, FP-02 (Hepsyn-B), AIC649, VGX-6200, KW-2, TomegaVax-HBV, ISA-204, NU-500, INX-102-00557, HBV MVA and PepTcell.

Preferably, the RNA interference therapeutic is a siRNA, a ddRNA or a shRNA. Preferably, the RNA interference therapeutic is selected from: TKM-HBV (ARB-1467), ARB-1740, ARC-520, ARC-521, BB-HB-331, REP-2139, ALN-HBV, ALN-PDL, LUNAR-HBV, GS3228836 and GS3389404.

Preferably, the immunomodulator is a TLR agonist. Preferably the TLR agonist is a TLR7, TLR8 or TLR9 agonist. Preferably, the TLR7, TLR8 or TLR9 agonist is selected from: RG7795 (RO-6864018), GS-9620, SM360320 (9-benzyl-8-hydroxy-2-(2-methoxy-ethoxy)adenine), AZD 8848 (methyl [3-({[3-(6-amino-2-butoxy-8-oxo-7,8-dihydro-9H-pyrin-9-yl)propyl][3-(4-morpholinyl)propyl]amino}methyl)phenyl]acetate) and ARB-1598.

Preferably, the RIG-I modulator is SB-9200. Preferably, the IL agonist or other immune acting protein is INO-9112 or recombinant IL12. Preferably, the immune checkpoint modulator/inhibitor is BMS-936558 (Opdivo (nivolumab)) or pembrolizumab. Preferably, the HBV entry inhibitor is Myrcludex B, IVIG-Tonrol or GC-1102.

Preferably, the cccDNA modulator is selected from: a direct cccDNA inhibitor, an inhibitor of cccDNA formation or maintenance, a cccDNA epigenetic modifier and an inhibitor of cccDNA transcription.

Preferably, the capsid assembly inhibitor/modulator, core or X protein targeting agent, direct cccDNA inhibitor, inhibitor of cccDNA formation or maintenance, or cccDNA epigenetic modifier is selected from: BAY 41-4109, NVR 3-778, GLS-4, NZ-4 (W28F), Y101, ARB-423, ARB-199, ARB-596, AB-506, JNJ-56136379, ASMB-101 (AB-V102), ASMB-103, CHR-101, CC-31326, AT-130 and R07049389.

Preferably, the interferon or modified interferon is selected from: interferon alpha (IFN-α), pegylated interferon alpha (PEG-IFN-α), interferon alpha-2a, recombinant interferon alpha-2a, peginterferon alpha-2a (Pegasys), interferon alpha-2b (Intron A), recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-λ), peginterferon lambda-1, interferon omega, interferon tau, interferon gamma (IFN-γ), interferon alfacon-1, interferon alpha-n1, interferon alpha-n3, albinterferon alpha-2b, BLX-883, DA-3021, PI 101 (also known as AOP2014), PEG-infergen, Belerofon, INTEFEN-IFN, albumin/interferon alpha 2a fusion protein, rHSA-IFN alpha 2a, rHSA-IFN alpha 2b, PEG-IFN-SA and interferon alpha biobetter. Particularly preferred are: peginterferon alpha-2a, peginterferon alpha-2b, glycosylated interferon alpha-2b, peginterferon beta-1a, and peginterferon lambda-1. More particularly preferred is peginterferon alpha-2a. Preferably, the HBV antiviral of distinct or unknown mechanism is selected from: AT-61 ((E)-N-(1-chloro-3-oxo-1-phenyl-3-(piperidin-1-yl)prop-1-en-2-yl)benzamide), AT130 ((E)-N-(1-bromo-1-(2-methoxyphenyl)-3-oxo-3-(piperidin-1-yl)prop-1-en-2-yl)-4-nitrobenzamide), analogues thereof, REP-9AC (REP-2055), REP-9AC′ (REP-2139), REP-2165 and HBV-0259. Preferably, the cyclophilin inhibitor is selected from: OCB-030 (NVP-018), SCY-635, SCY-575 and CPI-431-32.

Preferably, said HBV polymerase inhibitor is selected from: entecavir (Baraclude, Entavir), lamivudine (3TC, Zeffix, Heptovir, Epivir, and Epivir-HBV), telbivudine (Tyzeka, Sebivo), clevudine, besifovir, adefovir (hepsera), tenofovir. Preferably, tenofovir is in a salt form. Preferably, tenofovir is in a salt form selected from: tenofovir disoproxil fumarate (Viread), tenofovir alafenamide fumarate (TAF), tenofovir disoproxil orotate (DA-2802), tenofovir disopropxil aspartate (CKD-390), AGX-1009, and CMX157.

Preferably, the dinucleotide is SB9200. Preferably, the SMAC inhibitor is Birinapant. Preferably, the HDV targeting agent is Lonafamib.

Preferably, the HBV RNA destabilizer or other small-molecule inhibitor of HBV protein expression is RG7834 or AB-452.

Preferably, the at least one further therapeutic agent is an agent useful in the treatment and prevention of hepatitis B. Preferably, the at least one further therapeutic agent is an anti-HDV agent, an anti-HCV agent and/or an anti-HIV agent.

Preferably the at least one further therapeutic agent is selected from the group consisting of: HBV polymerase inhibitor, interferon, viral entry inhibitor, BAY 41-4109, reverse transcriptase inhibitor, a TLR-agonist, AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and a combination thereof, wherein the HBV polymerase inhibitor is preferably at least one of Lamivudine, Entecavir, Tenofovir, Adefovir, Telbivudine, Clevudine; and wherein the TLR agonist is preferably selected from the group consisting of SM360320 (9-benzyl-8-hydroxy-2-(2-methoxy-ethoxy)adenine), AZD 8848 (methyl [3-({[3-(6-amino-2-butoxy-8-oxo-7,8-dihydro-9H-purin-9-yl)propyl][3-(4-morpholinyl)propyl]amino}methyl)phenyl] acetate) and a combination thereof.

Preferably, the compound of the invention is for use in combination with one, two or more further therapeutic agent(s) as defined above.

Preferably, the pharmaceutical composition of the invention comprises one, two or more further therapeutic agent(s) as defined above.

The pharmaceutical composition defined above is preferably intended for use in the treatment and/or prevention of an HBV infection and/or a condition related to an HBV infection. Preferably, the pharmaceutical composition of the invention is for use in treating, eradicating, reducing, slowing or inhibiting an HBV infection in an individual in need thereof, and/or in reducing the viral load associated with an HBV infection in an individual in need thereof, and/or in reducing reoccurrence of an HBV infection in an individual in need thereof, and/or in inducing remission of hepatic injury from an HBV infection in an individual in need thereof, and/or in prophylactically treating an HBV infection in an individual afflicted with a latent HBV infection.

In an embodiment, the invention provides a kit comprising at least one pharmaceutically acceptable vial or container containing one or more doses of a compound of the invention or of a pharmaceutical composition of the invention and optionally a) instructions for use thereof in mammals and/or b) an infusion bag or container containing a pharmaceutically acceptable diluent. It is a further object of the invention a process for the synthesis of a compound of general formula (I) or (Ia).

In particular, it is a further object of the invention a process for the synthesis of the compound of formula I or the pharmaceutically acceptable salt, tautomer, solvate, isomer or stereoisomer thereof as defined above, said process comprising at least one of the following steps:

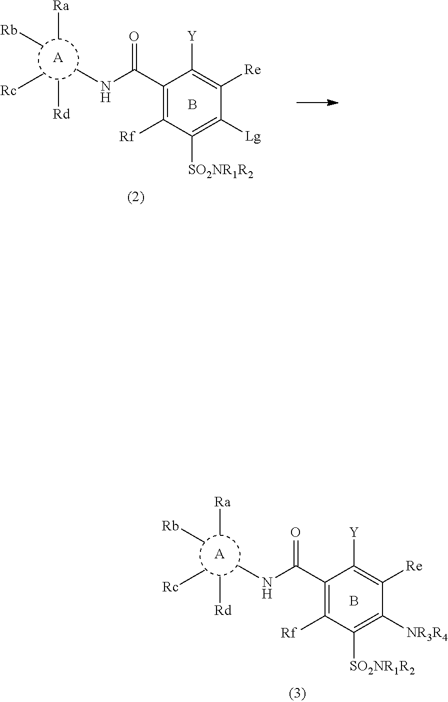

reacting a compound of formula (2) with an amine of formula NHR₃R₄ to obtain a compound of formula (3), wherein A, B, Ra, Rb, Rc, Rd, Re, Rf, Y, R₁, R₂, R₃ and R₄ are as defined above, and Lg is a leaving group such as Cl or F;

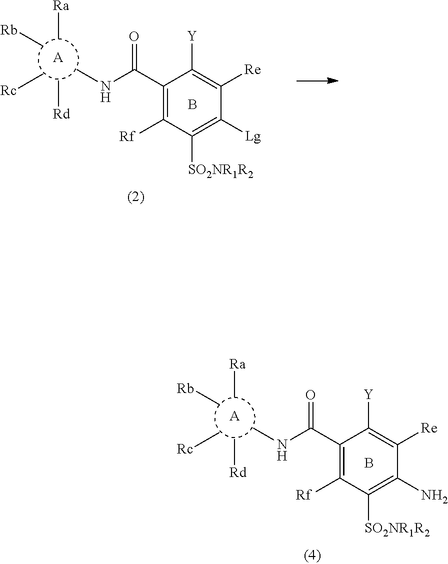

reacting a compound of formula (2) with an ammonium salt such as NH₄OH to obtain a compound of formula (4), wherein A, B, Ra, Rb, Rc, Rd, Re, Rf, Y, R₁ and R₂ are as defined above, and Lg is a leaving group such as Cl or F;

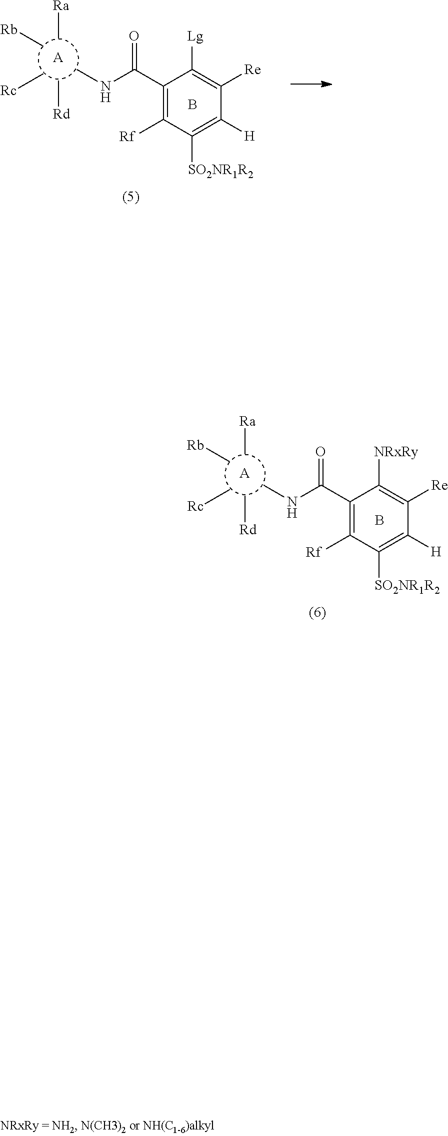

reacting a compound of formula (5) with an amine of formula (CH₃)₂NH or (C₁₋₆)alkylNH₂ or with NH₄OH to obtain a compound of formula (6) wherein A, B, Ra, Rb, Rc, Rd, Re, Rf, R₁ and R₂ are as defined above and Lg is a leaving group such as Cl or F.

It is a further object of the invention a pharmaceutical composition comprising an effective amount of one or more compounds as defined above or a pharmaceutically acceptable prodrug thereof, alone or in combination with other active compounds, and at least one pharmaceutically acceptable excipient.

The present invention includes within its scope prodrugs of the compounds of formula (I) or (Ia) above. In general, such prodrugs will be functional derivatives of the compounds of the invention which are readily convertible in vivo into the required compound of formula (I) or (Ia). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The invention also includes all suitable isotopic variations of a compound of the disclosure. Examples of isotopes that can be incorporated into compounds of the disclosure include isotopes such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the disclosure, for example, those in which a radioactive isotope such as ³H or ¹⁴C is incorporated, are useful in drug and/or substrate tissue distribution studies. Further, substitution with isotopes such as deuterium ²H, may afford certain therapeutic advantages resulting from greater metabolic stability. Isotopic variations of the compounds of the disclosure can generally be prepared by conventional procedures such as by the illustrative methods or by the preparations described in the examples hereafter using appropriate isotopic variations of suitable reagents. The present invention includes within its scope solvates of the compounds of formula (I) or (Ia) or of the relative salts, for example, hydrates, alcoholates and the like.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention.

Pure stereoisomeric forms of the compounds and intermediates of this invention may be obtained by the application of art-known procedures and are intended to be encompassed by the scope of the invention. In particular, “pure stereoisomeric form” or “stereoisomerically pure” indicate a compound having stereoisomeric excess of at least 80%, preferably of at least 85%. For instance, enantiomers may be separated from each other by the selective crystallization of their diastereomeric salts or by chromatographic techniques using chiral stationary phases. Pure stereochemically isomeric forms may also be derived from the corresponding pure stereochemically isomeric forms of the appropriate starting materials, provided that the reaction occurs stereospecifically. The term “enantiomerically pure” shall be interpreted in a similar way, having regard to the enantiomeric ratio.

In addition, the compounds disclosed herein may exist as tautomers and all tautomeric forms are intended to be encompassed by the scope of the invention, even though only one tautomeric structure is depicted.

The compounds may exist in different isomeric forms, all of which are encompassed by the present invention. For example, specific compounds of the invention may exist as cis and trans geometric isomers, and all are encompassed by the invention.

When any variable (e.g. R₃ and R₄, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” should be taken to be equivalent to the phrase “unsubstituted or substituted with one or more substituents” and in such cases the preferred embodiment will have from zero to three substituents. More particularly, there are zero to two substituents.

The skilled person will readily understand the meaning of the term “absent” referring to a given substituent. In particular, it will be understood that such term applies when the atom to which the substituent would be bound has reached its maximum valency and thus cannot accommodate any further substituent. For instance, a substituent may be absent when the atom to which it would be bound is involved in a multiple bond.

It is also understood that the expression “is absent” (referring to a given substituent) means that the atom to which the substituent would be bound is a heteroatom, preferably nitrogen, which is comprised in a heteroaryl ring, such as a pyridine or a pyrimidine ring.

The expressions “one or more substituents” refer in particular to 1, 2, 3, 4 or more substituents, in particular to 1, 2, 3 or 4 substituents, more in particular to 1, 2 or 3 substituents.

As used herein, “alkyl” is intended to include both branched and straight-chain saturated aliphatic hydrocarbon groups having the specified number of carbon atoms. For example, “C₁₋₆alkyl” is defined to include groups having 1, 2, 3, 4, 5 or 6 carbons in a linear or branched arrangement. For example, “C₁₋₆ alkyl” specifically includes methyl, ethyl, n-propyl, i-propyl, n-butyl, t-butyl, i-butyl, pentyl, hexyl, and so on. “C₁₋₄alkyl” is defined to include groups having 1, 2, 3 or 4 carbons in a linear or branched arrangement. “C₁₋₃alkyl” is defined to include groups having 1, 2, or 3 carbons in a linear or branched arrangement. Preferred alkyl groups are methyl, ethyl, i-propyl or t-butyl.

As used herein, “alkoxy” represents an alkyl group of indicated number of carbon atoms attached through an oxygen bridge. “Alkoxy” therefore encompasses the definitions of alkyl above. C₁₋₆ alkoxy group is preferably a linear or branched C₁₋₄ alkoxy group, more preferably a C₁₋₃ alkoxy group, still more preferably a C₁₋₂ alkoxy group. Examples of suitable alkoxy groups include, but are not limited to methoxy, ethoxy, n-propoxy, i-propoxy, n-butoxy, s-butoxy or t-butoxy. The preferred alkoxy group is methoxy.

As used herein, the terms “haloC₁₋₆alkyl” and “haloC₁₋₆alkoxy” mean a C₁₋₆alkyl or C₁₋₆alkoxy group in which one or more (in particular, 1 to 3) hydrogen atoms have been replaced by halogen atoms, especially fluorine or chlorine atoms. HaloC₁₋₆alkoxy group is preferably a linear or branched haloC₁₋₄alkoxy group, more preferably a haloC₁₋₃alkoxy group, still more preferably a haloC₁₋₂alkoxy group, for example OCF₃, OCHF₂, OCH₂F, OCH₂CH₂F, OCH₂CHF₂ or OCH₂CF₃, and most especially OCF₃ or OCHF₂. HaloC₁₋₆alkyl group is preferably a linear or branched haloC₁₋₃alkyl group, more preferably a haloC₁₋₂alkyl group for example, CF₃, CHF₂, CH₂F, CH₂CH₂F, CH₂CHF₂, CH₂CF₃ or CH(CH₃)CF₃, and most especially CF₃, CHF₂ or CH(CH₃)CF₃.

As used herein, the term “C₁₋₆hydroxyalkyl” means a C₁₋₆alkyl group in which one or more (in particular, 1 to 3) hydrogen atoms have been replaced by hydroxy groups. Similarly, the term “hydroxyC₁₋₄alkyl” means a C₁₋₄alkyl group in which one or more (in particular, 1 to 2) hydrogen atoms have been replaced by hydroxy groups. Illustrative examples include, but are not limited to CH₂OH, CH₂CH₂OH, CH(CH₃) OH and CHOHCH₂OH.

As used herein, the term “aryl” means a monocyclic or polycyclic aromatic ring comprising carbon atoms and hydrogen atoms. If indicated, such aromatic ring may include one or more heteroatoms, then also referred to as “heteroaryl” or “heteroaromatic ring”, preferably, 1 to 3 heteroatoms, independently selected from nitrogen, oxygen, and sulfur, preferably nitrogen. As is well known to those skilled in the art, heteroaryl rings have less aromatic character than their all-carbon counter parts. Thus, for the purposes of the present invention, a heteroaryl group need only have some degree of aromatic character. Illustrative examples of aryl groups are optionally substituted phenyl. Illustrative examples of heteroaryl groups according to the invention include optionally substituted thiophene, oxazole, thiazole, thiadiazole, imidazole, pyrazole, pyrimidine, pyrazine and pyridine. Thus, examples of monocyclic aryl optionally containing one or more heteroatoms, for example one or two heteroatoms, are a 5- or 6-membered aryl or heteroaryl group such as, but not limited to, phenyl, pyridyl, pyrimidinyl, pyrazinyl, pyridazinyl, pyrrolyl, thienyl, thiazolyl, thiadiazolyl, pyrazolyl, imidazolyl, triazolyl, tetrazolyl, furyl, isoxazolyl, oxadiazolyl and oxazolyl. Examples of polycyclic aromatic ring, optionally containing one or more heteroatoms, for example one or two heteroatoms, are a 8-10 membered aryl or heteroaryl group such as, but not limited to, benzimidazolyl, benzofurandionyl, benzofuranyl, benzofurazanyl, benzopyrazolyl, benzotriazolyl, benzothienyl, benzoxazolyl, benzoxazolonyl, benzothiazolyl, benzothiadiazolyl, benzodioxolyl, benzoxadiazolyl, benzoisoxazolyl, benzoisothiazolyl, indolyl, indolizinyl, isoindolinyl, indazolyl, isobenzofuranyl, isoindolyl, isoquinolyl, quinazolinyl, quinolyl, quinoxalinyl, quinolizinyl, naphtyl, naphthyridinyl and phthalazinyl. A preferred aromatic ring according to the present invention is phenyl. A preferred heteroaromatic ring according to the present invention is pyridyl.

Heterocycle, heterocyclic compound or ring structure is a cyclic compound that has atoms of at least two different elements as members of its ring(s).

As used herein, the term “heterocyclic ring” is a saturated or partially saturated non aromatic monocyclic or bicyclic ring system, of 3 to 10 members which contains one or more heteroatoms selected from N, O or S. Examples include, but are not limited to azetidinyl, piperazinyl, piperidinyl, morpholinyl, thiomorpholinyl, thiazolidinyl, pyrrolidinyl, azepanyl, diazepanyl, oxazepanyl, thiazepanyl, azocanyl, oxazocanyl, hexahydrofuro[2,3-b]furanyl or octahydrocyclopenta[b]pyrrole.

A substituent on a saturated, partially saturated or unsaturated heterocycle can be attached at any substitutable position.

As used herein, the terms “C₃₋₇cycloalkyl”, “C₃₋₁₀cylcloalkyl respectively” mean saturated cyclic hydrocarbon (cycloalkyl) with 3, 4, 5, 6 or 7 or with 3, 4, 5, 6, 7, 8, 9 or 10 carbon atoms and are respectively generic to cyclopropyl, cyclobutyl, cyclopentyl, cyclohexyl, cycloheptyl and to cyclopropyl, cyclobutyl, cyclopentyl, cyclohexyl, cycloheptyl, cyclooctyl, cyclononyl or cyclodecyl. Said saturated ring optionally contains one or more heteroatoms (also referred to as heterocyclyl, C₃₋₁₀heterocycloalkyl, heterocyclic ring or heterocycloalkyl), such that at least one carbon atom is replaced by a heteroatom selected from N, O and S, in particular from N and O. Depending on the dimension of the ring, it can be of a cyclic or bicyclic ring structure. Examples include, but are not limited to oxetanyl, azetidinyl, tetrahydro-2H-pyranyl, piperazinyl, piperidinyl, tetrahydrofuranyl, morpholinyl, thiomorpholinyl, thiazolidinyl, thiolane 1,1-dioxide, pyrrolidinyl, azepanyl, diazepanyl, oxazepanyl, thiazepanyl, azocanyl or oxazocanyl. Preferred are saturated cyclic hydrocarbons with 3, 4 or 5 carbon atoms and 1 oxygen or 1 nitrogen atom. Examples include oxetanyl, tetrahydrofuranyl, tetrahydro-2H-pyranyl, piperidinyl or pyrrolidinyl. It should be noted that different isomers of the various heterocycles may exist within the definitions as used throughout the specification. For example, pyrrolyl may be 1H-pyrrolyl or 2H-pyrrolyl.

It should also be noted that the radical positions on any molecular moiety used in the definitions may be anywhere on such moiety as long as it is chemically stable. For example, pyridyl includes 2-pyridyl, 3-pyridyl, 4-pyridyl.

As used herein, the term “halogen” includes fluorine, chlorine, bromine and iodine, of which fluorine, chlorine and bromine are preferred.

The term “heteroatom” refers to an atom other than carbon or hydrogen in a ring structure or a saturated backbone as defined herein. Typical heteroatoms include N(H), O, S.

Included in the instant invention is the free base of compounds of formula (I) or (Ia), as well as the pharmaceutically acceptable salts and stereoisomers thereof. Some of the specific compounds exemplified herein are the protonated salts of amine compounds. Compounds of formula (I) or (Ia) containing one or more N atoms may be protonated on any one, some or all of the N atoms. The term “free base” refers to the amine compounds in non-salt form. The encompassed pharmaceutically acceptable salts not only include the salts exemplified for the specific compounds described herein, but also all the typical pharmaceutically acceptable salts of the free form of compounds of formula (I) or (Ia). The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base. In a preferred embodiment, the compounds of the invention have at least one acidic proton and the corresponding sodium or potassium salt can be formed, for example, by reaction with the appropriate base.

Thus, pharmaceutically acceptable salts of the compounds of this invention include the conventional non-toxic salts of the compounds of this invention as formed by reacting a basic instant compound with an inorganic or organic acid or an acid compound with an inorganic or organic base. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional non-toxic salts further include those derived from an inorganic base, such as potassium, sodium hydroxide, magnesium or calcium hydroxide, as well as salts prepared from organic bases, such as ethylene diamine, lysine, tromethamine, meglumine and the like. Preferably, a pharmaceutically acceptable salt of this invention contains one equivalent of a compound of formula (I) or (Ia) and 1, 2 or 3 equivalent of an inorganic or organic acid or base. More particularly, pharmaceutically acceptable salts of this invention are the tartrate, trifluoroacetate or the chloride salts.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N′-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The compounds of the present invention find use in a variety of applications for human and animal health. The compounds of the present invention are inhibitors of hepatitis B virus (HBV).

In the context of the present invention, HBV may be any known isoltate, genotype, strain, etc. of HBV.

In particular, the hepatitis B virus has been classified into eight main genotypes (designated A-H), and two additional genotypes (I and J) were tentatively proposed. HBV genotypes have been further separated into several subgenotypes that differ by 4.0 to 7.5% in the whole nucleotide sequence. HBV genotypes differ substantially in many virological and probably some clinical parameters; however, the precise role of HBV genotypes in the evolution of the infection remains controversial. Due to geographical distribution, only two or three HBV genotypes co-circulate in most regions of the world, thereby limiting genotype comparisons.

The compounds of the present invention are inhibitors of hepatitis B virus (HBV) useful for the treatment and/or prevention of an HBV infection. In particular the compounds of the present invention are inhibitors of hepatitis B virus (HBV) core (HBc) protein useful for the treatment and/or prevention of an HBV infection.

The compounds, compositions and methods provided herein are particularly deemed useful for treating, ameliorating or preventing HBV infection and related conditions, including chronic hepatitis B, HBV/HDV co-infection, HBV/HCV co-infection, HBV/HIV co-infection, inflammation, necrosis, cirrhosis, hepatocellular carcinoma, hepatic decompensation and hepatic injury from an HBV infection.

In the present invention, the expression “HBV infection” comprises any and all conditions deriving from infection with HBV, including but not limited to hepatitis B, preferably chronic hepatitis B, HBV/HDV co-infection, HBV/HCV coinfection, HBV/HIV co-infection.

HBV infection leads to a wide spectrum of hepatic complications, all of these are intended as conditions related to an HBV infection. As used herein, “condition related to an HBV infection” is preferably selected from the group consisting of: chronic hepatitis B, HBV/HDV co-infection, HBV/HCV co-infection, HBV/HIV co-infection, inflammation, necrosis, cirrhosis, hepatocellular carcinoma, hepatic decompensation and hepatic injury from an HBV infection.

Expressions like “treating, eradicating, reducing, slowing or inhibiting an HBV infection” are used to indicate the application or administration of a therapeutic agent, i.e., a compound of the invention (alone or in combination with another pharmaceutical agent), to a patient or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

Efficacy of treatment may be determined using quantification of viral load or other evidence of infection, such as through measurement of HBeAg, HBsAg, HBV DNA levels, ALT activity levels, serum HBV levels, and the like, thereby allowing adjustment of treatment dose, treatment frequency, and treatment length.

HBeAg stands for hepatitis B e-antigen. This antigen is a protein from the hepatitis B virus that circulates in infected blood when the virus is actively replicating.

ALT stands for Alanine Transaminase and is an enzyme involved in the transfer of an amino group from the amino acid alanine to alpha-ketoglutaric acid to produce glutamate and pyruvate. ALT is located primarily in liver and kidney, with lesser amounts in heart and skeletal muscle. ALT is commonly measured clinically as part of liver function tests.

The compounds of the invention can reduce viral load in an individual suffering from an HBV infection. In a non limiting embodiment, the compounds of the invention result in viral load reduction during therapy in an individual in need thereof from a minimum of one- or two-log decrease to a maximum of about eight-log decrease.

As used herein, the expression “remission of hepatic injury from an HBV infection” means that the chronic necroinflammatory liver disease has been halted by the fact that the viral antigens have disappeared from the organ (and the immune system no longer attacks the liver cells).

As used herein, the term “prophylactically treating” means no disorder or disease development if none had occurred, or no further disorder or disease development if there had already been development of the disorder or disease. Also considered is the ability to prevent some or all of the symptoms associated with the disorder or disease. An example of prophylactic treatment might also indicate the necessity of reducing the risk of infecting a liver graft (in case of liver transplant in chronically infected patients) or infecting newborns (in case of chronically infected mothers that pass the virus at time of delivery).

As used herein, “reducing reoccurrence of an HBV infection” indicates that patients may have reactivation of HBV replication and exacerbation of a condition related to an HBV infection, e.g. hepatitis, after years of quiescence. These patients may still be at risk of developing a condition related to an HBV infection, e.g. hepatocellular carcinoma development. Antiviral therapy is also recommended as prophylaxis for patients who are HBsAg-positive as well as patients who are HBsAg-negative and hepatitis B core antibody-positive who require treatment with immunosuppressive therapies that are predicted to have a moderate to high risk of HBV reactivation.

The compounds of this invention may be administered to mammals, preferably humans, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

The invention also provides pharmaceutical compositions comprising one or more compounds of this invention and a pharmaceutically acceptable carrier. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants. Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulstion.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of formula (I) or (Ia) may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of formula (I) or (Ia) are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The compounds of the invention may be presented in a liposome or other micro particulate or other nanoparticle designed to target the compound. Acceptable liposomes can be neutral, negatively, or positively charged, the charge being a function of the charge of the liposome components and pH of the liposome solution. Liposomes can be normally prepared using a mixture of Phospholipids and cholesterol. Suitable phospholipids include phosphatidylcholine, phosphatidylethanolamine, phosphatidic acid, phosphotidylglycerol, phosphatidylinositol. Polyethylene glycol can be added to improve the blood circulation time of liposomes. Acceptable nanoparticles include albumin nanoparticles and gold nanoparticles.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing anti HBV treatment. Administration generally occurs in an amount between about 0.01 mg/kg of body weight to about 100 mg/kg of body weight per day, preferably between about 0.01 mg/kg of body weight to about 60 mg/kg of body weight per day, preferably between about 0.1 mg/kg of body weight to about 50 mg/kg of body weight per day, preferably between about 0.5 mg/kg of body weight to about 40 mg/kg of body weight per day.

The instant compounds are also useful in combination with known therapeutic agents for simultaneous, separate or sequential administration.

In an embodiment, the compounds of the present invention may be used in combination with at least one or more additional therapeutic agents, in particular anti-HBV agents.

The indication that compounds of the invention are for use in the treatment and/or prevention of an HBV infection indicates that the compounds are efficacious for treating, eradicating, reducing, slowing or inhibiting an HBV infection.

The therapeutic agent is any agent commonly used in the treatment and/or prevention and/or amelioration of an HBV infection or a condition related to an HBV infection. The therapeutic agent is known in the art.

The term “anti-HBV agent”, or more simply “HBV antiviral(s)” also includes compounds that are therapeutic nucleic acids, antibodies or proteins either in their natural form or chemically modified and/or stabilized. The term therapeutic nucleic acid includes but is not limited to nucleotides and nucleosides, oligonucleotides, polynucleotides, of which non limiting examples are antisense oligonucleotides, miRNA, siRNA, shRNA, therapeutic vectors and DNA/RNA editing components.

The term anti-HBV agent also includes compounds capable of treating HBV infection via immunomodulation, i.e. immunomodulators or immunomodulating compounds. Examples of immunomodulators are interferon-α (IFN-α), pegylated interferon-α or stimulants of the innate immune system such as Toll-like receptor 7 and/or 8 agonists and therapeutic or prophylactic vaccines. One embodiment of the present invention relates to combinations of a compound of formula (I) or (Ia) or any subgroup thereof, as specified herein, with an immunomodulating compound, more specifically a Toll-like receptor 7 and/or 8 agonist.

The additional HBV antiviral(s) can be selected for example, from therapeutic vaccines; RNA interference therapeutic/antisense oligonucleotides (e.g. siRNA, ddRNA, shRNA); immunomodulators (such as TLR agonists (e.g. TLR7, TLR8 or TLR9 agonists); STING agonists; RIG-I modulators; NKT modulators; IL agonists; Interleukin or other immune active proteins, therapeutic and prophylactic vaccines and immune checkpoint modulators; HBV entry inhibitors; cccDNA modulators (such as for example direct cccDNA inhibitors, inhibitors of cccDNA formation or maintenance, cccDNA epigenetic modifiers, inhibitors of cccDNA transcription); inhibitors of HBV protein espression; agents targeting HBV RNA; capsid assembly inhibitors/modulators; core or X protein targeting agents; nucleotide analogues; nucleoside analogues; interferons or modified interferons; HBV antivirals of distinct or unknown mechanism; cyclophilin inhibitors; sAg release inhibitors; HBV polymerase inhibitors; dinucleotides; SMAC inhibitors; HDV targeting agents; viral maturation inhibitors; reverse transcriptase inhibitors and HBV RNA destabilizers and other small-molecule inhibitors of HBV protein expression.

In particular, the combination of previously known anti-HBV agents, such as interferon-α (IFN-α), pegylated interferon-α, 3TC, tenofovir, lamivudine, entecavir, telbivudine, and adefovir or a combination thereof, and a compound of formula (I) or (Ia) or any subgroup thereof can be used as a medicine in a combination therapy. Additional examples of further therapeutic agents that may be combined with the compounds of the present invention include: Zidovudine, Didanosine, Zalcitabine, Stavudine, Abacavir, ddA, Emtricitabine, Apricitabine, Atevirapine, ribavirin, acyclovir, valacyclovir, famciclovir, ganciclovir, valganciclovir, cidofovir, Efavirenz, Nevirapine, Delavirdine and Etravirine.

Particular examples of such HBV antiviral(s) include, but are not limited to:
RNA interference (RNAi) therapeutics: TKM-HBV (also known as ARB-1467), ARB-1740, ARC-520, ARC-521, BB-HB-331, REP-2139, ALN-HBV, ALN-PDL, LUNAR-HBV, GS3228836, and GS3389404;
HBV entry inhibitors: Myrcludex B, IVIG-Tonrol, GC-1102;
HBV capsid inhibitor/modulators, core or X protein targeting agents, direct cccDNA inhibitors, inhibitors of cccDNA formation or maintenance, or cccDNA epigenetic modifiers: BAY 41-4109, NVR 3-778, GLS-4, NZ-4 (also known as W28F), Y101, ARB-423, ARB-199, ARB-596, AB-506, JNJ-56136379, ASMB-101 (also known as AB-V102), ASMB-103, CHR-101, CC-31326, AT-130, R07049389.
HBV polymerase inhibitors: entecavir (Baraclude, Entavir), lamivudine (3TC, Zeffix, Heptovir, Epivir, and Epivir-HBV), telbivudine (Tyzeka, Sebivo), clevudine, besifovir, adefovir (hepsera), tenofovir (in particular tenofovir disoproxil fumarate (Viread), tenofovir alafenamide fumarate (TAF)), tenofovir disoproxil orotate (also known as DA-2802), tenofovir disopropxil aspartate (also known as CKD-390), AGX-1009, and CMX157);
HBV RNA destabilizers and other small-molecule inhibitors of HBV protein expression: RG7834, AB-452;
cyclophilin inhibitors: OCB-030 (also known as NVP-018), SCY-635, SCY-575, and CPI-431-32;
dinucleotides: SB9200;
compounds of distinct or unknown mechanism, such as but not limited to AT-61 ((E)-N-(1-chloro-3-oxo-1-phenyl-3-(piperidin-1-yl)prop-1-en-2-yl)benzamide), AT130 ((E)-N-(1-bromo-1-(2-methoxyphenyl)-3-oxo-3-(piperidin-1-yl)prop-1-en-2-yl)-4-nitrobenzamide), and similar analogs; REP-9AC (also known as REP-2055), REP-9AC′ (also known as REP-2139), REP-2165 and HBV-0259;
TLR agonists (TLR7, 8 and/or 9): RG7795 (also known as RO-6864018), GS-9620, SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-pyrin-9-yl)propyl][3-(4-morpholinyl)propyl]amino}methyl)phenyl]acetate); ARB-1598;
RIG-I modulators: SB-9200;
SMAC inhibitor: Birinapant;
Immune Check Point inhibitors: BMS-936558 (Opdivo (nivolumab)), KEYTRUDA® (pembrolizumab);
therapeutic vaccines: HBsAG-HBIG, HB-Vac, ABX203, NASVAC, GS-4774, GX-110 (also known as HB-110E), CVI-HBV-002, RG7944 (also known as INO-1800), TG-1050, FP-02 (Hepsyn-B), AIC649, VGX-6200, KW-2, TomegaVax-HBV, ISA-204, NU-500, INX-102-00557 HBV MVA, PepTcell;
IL agonists and immune acting proteins: INO-9112; recombinant IL12;
interferons: interferon alpha (IFN-α), interferon alpha-2a, recombinant interferon alpha-2a, peginterferon alpha-2a (Pegasys), interferon alpha-2b (Intron A), recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-λ), peginterferon lambda-1, interferon omega, interferon tau, interferon gamma (IFN-γ), interferon alfacon-1, interferon alpha-n1, interferon alpha-n3, albinterferon alpha-2b, BLX-883, DA-3021, PI 101 (also known as AOP2014), PEG-infergen, Belerofon, INTEFEN-IFN, albumin/interferon alpha 2a fusion protein, rHSA-IFN alpha 2a, rHSA-IFN alpha 2b, PEG-IFN-SA, interferon alpha biobetter; in particular, peginterferon alpha-2a, peginterferon alpha-2b, glycosylated interferon alpha-2b, peginterferon beta-1a, and peginterferon lambda-1; more in particular, peginterferon alpha-2a;
HDV targeting agent: Lonafamib.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In some embodiments, pulsed administration is more effective than continuous treatment because total pulsed doses are often lower than would be expected from continuous administration of the same composition. Each pulse dose can be reduced and the total amount of drug administered over the course of treatment is minimized. Individual pulses can be delivered to the patient continuously over a period of several hours, such as about 2, 4, 6, 8, 10, 12, 14 or 16 hours, or several days, such as 2, 3, 4, 5, 6 or 7 days.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The present invention will be described by means of the following non-limiting examples and biological data are presented.

### Materials and Methods

### Chemistry

### General

Unless otherwise indicated, commercially available reagents and solvents (HPLC grade) were used without further purification.

Specifically, the following abbreviations may have been used in the descriptions of the experimental methods:

NMR: Nuclear Magnetic Resonance; ¹H: proton; MHz: Megahertz; Hz: Hertz; HPLC: High Performance Liquid Chromatography; LC-MS: Liquid Chromatography Mass Chromatography Spectrum; s: second(s); min: minute(s); h: hour(s); mg: milligram(s); g: gram(s); Ml: microliter(s); mL: millilitre(s); mmol: millimole(s); nm: nanometer(s) μM: micromolar; M: molarity or molar concentration; Rt: retention time in minutes; MW: microwave; Boc: tert-butyloxycarbonyl protecting group; DMF: dimethylformamide; DMSO: dimethylsulfoxide; MeOH: methanol; EtOH: ethanol; EtOAc: ethyl acetate; DCM: dichloromethane; MeCN: Acetonitrile; PE: Petroleum Ether; TFA: trifluoroacetic acid; (g): gas; eq.: equivalent(s); RT: room temperature; DIPEA: N,N-diisopropylethylamine; DIAD: Diisopropyl azodicarboxylate; sat.aq.: saturated aqueous solution; TEA: triethylamine; THF: tetrahydrofuran; IPA: isopropylamine; pTSA: para toluene sulfonic acid; TBDMS: tert-butyldimethylsilyl; LiHMDS: Lithium bis(trimehtylsilyl)amide; TBTU: 2-(1H-Benzotriazole-1-yl)-1,1,3,3-tetramethylaminium tetrafluoroborate.

Except where indicated otherwise, all temperatures are expressed in ° C. (degrees centigrade) or K (Kelvin).

The ¹H-NMR spectra were acquired with an Avance II 300 MHz Bruker spectrometer. The chemical shifts are expressed in parts per million (ppm, δ units). The coupling constants are expressed in Hertz (Hz) and the splitting patterns are described as s (singlet), bs (broad signal), d (doublet), t (triplet), q (quartet), quint (quintet), m (multiplet).

The LC-MS analyses were performed by means of an UPLC Acquity Waters System equipped with the SQD spectrometer, single quadrupole mass detector, and a TUV detector, using column 1: ACQUITY UPLC BEH SHIELD, RP₁₈ (2.1×50 mm, id=1.7 μm); column 2: ACQUITY UPLC HSS T3, RP₁₈ (2.1×50 mm, id=1.8 μm) and column 3: ACQUITY UPLC BEH SHIELD, RP₁₈ (2.1×100 mm, id=1.7 μm). Column temperature 40° C. Sample temperature 25° C. Phase A was composed by water (HiPerSolv Chromanorm Water VWR for HPLC-MS)+0.05% Trifluoroacetic Acid; Phase B by CH₃CN (HiPerSolv Chromanorm Acetonitrile SuperGradient VWR, suitable for UPLC/UHPLC instruments)+0.05% Trifluoroacetic Acid; flow rate: 0.5 mL/min; UV detection (DIODE array) 200 nm; ESI+ and ESI− detection in the 100-1000 m/z range.

Method 1: column 1, run time: 3 minutes, run gradient: 5% B to 100% B in 2.80 min+100% B for 0.2 min, equilibration time: 0.8 min, ionization mode: ESI⁺.

Method 2: column 2, run time: 4 minutes, run gradient: 0% B to 45% B in 3.5 min+45% B to 100% B in 0.05 min+100% B for 0.45 min, equilibration time: 0.8 min, ionization mode: ESI⁺.

Method 3: column 3, run time: 6 minutes, run gradient: 5% B to 100% B in 5 min+100% B for 1 min, equilibration time: 2 min.

Method 4: column 3, run time: 6 minutes, run gradient: 5% B to 50% B in 5 min+50% B to 100% B in 0.2 min 100% B for 0.8 min, equilibration time: 2 min, ionization mode: ESI⁺.

Method 5: column 1, run time: 3 minutes, run gradient: 5% B to 100% B in 2.80 min+100% B for 0.2 min, equilibration time: 0.8 min, ionization mode: ESI⁺.

Method 6: column 2, run time: 4 minutes. run gradient: 0% B to 45% B in 3.5 min+45% B to 100% B in 0.05 min+100% B for 0.45 min. Equilibration time: 0.8 min, ionization mode: ESI⁺.

Method 7: column 3, run time: 6 minutes, run gradient: 5% B to 100% B in 5 min+100% B for 1 min, equilibration time: 2 min, ionization mode: ESI⁺.

Method 8: column 3, run time: 6 minutes, run gradient: 5% B to 50% B in 5 min+50% B to 100% B in 0.2 min 100% B for 0.8 min, Equilibration time: 2 min, ionization mode: ESI⁺.

Method 9: column 1. run time: 4 minutes, column 1, run time: 4 minutes, run gradient:5% B to 100% B in 3.00 min+100% B for 1 min, equilibration time: 0.8 min, ionization mode: ESI⁺.

Method 10: column 1. run time: 4 minutes, run gradient: 5% B to 100% B in 3.00 min+100% B for 1 min, equilibration time: 0.8 min, Ionization Mode: EST⁻.

Method 11: column 1, run time: 3 minutes, run gradient: 40% B to 100% B in 2.80 min+100% B for 0.2 min, equilibration time: 0.8 min. Ionization Mode: ESI⁺.

Method 12: column 3, run time: 6 minutes, run gradient: 25% B to 70% B in 5 min+100% B for 1 min, equilibration time: 2 min, Flow: 0.5 mL/min, ionization mode: ESI⁺.

### Synthesis

According to a further aspect of the invention there is provided a process for the preparation of compounds of formula (I), (Ia) or salts thereof. The following schemes are examples of synthetic schemes that may be used to synthesise the compounds of the invention. In the following schemes reactive groups can be protected with protecting groups and deprotected according to well established techniques.

In the following schemes unless otherwise indicated A, B, Y, R₁, R₂, R₃, R₄, R₅, Ra, Rb, Rc, Rd, Re, Rf are as defined herein above in formula (I) or (Ia).

It will be understood by those skilled in the art that certain compounds of the invention can be converted into other compounds of the invention according to standard chemical methods. Unless otherwise indicated, compounds of the invention can be prepared following general procedures according to Scheme 1:

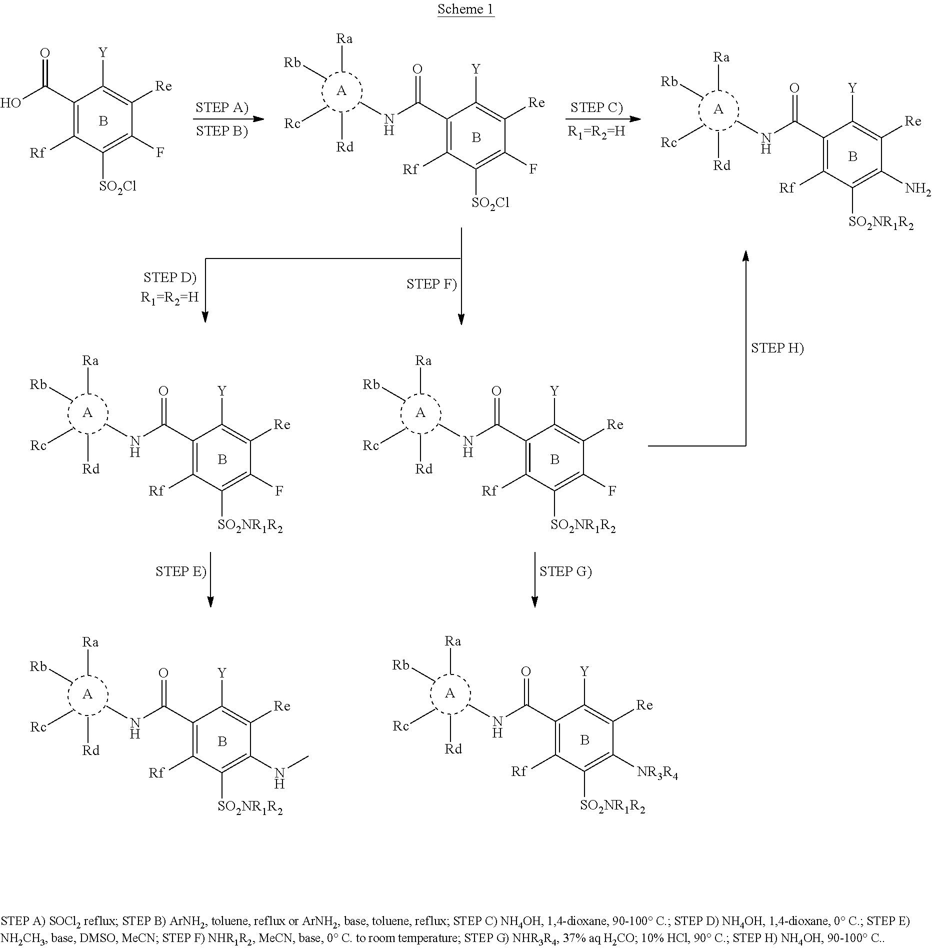

Example E36 can be prepared according to Scheme 2:

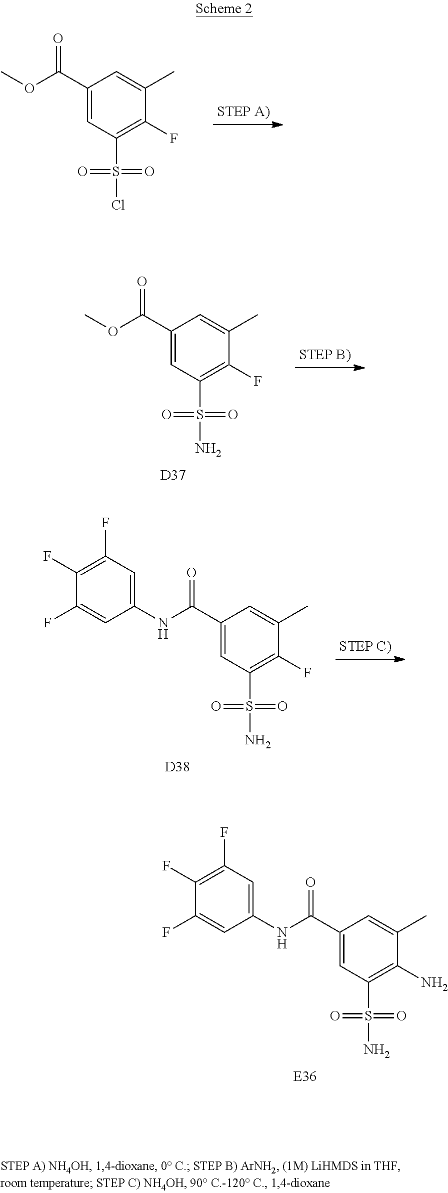

Examples E37 and E58 can be prepared according to the following Scheme 3:

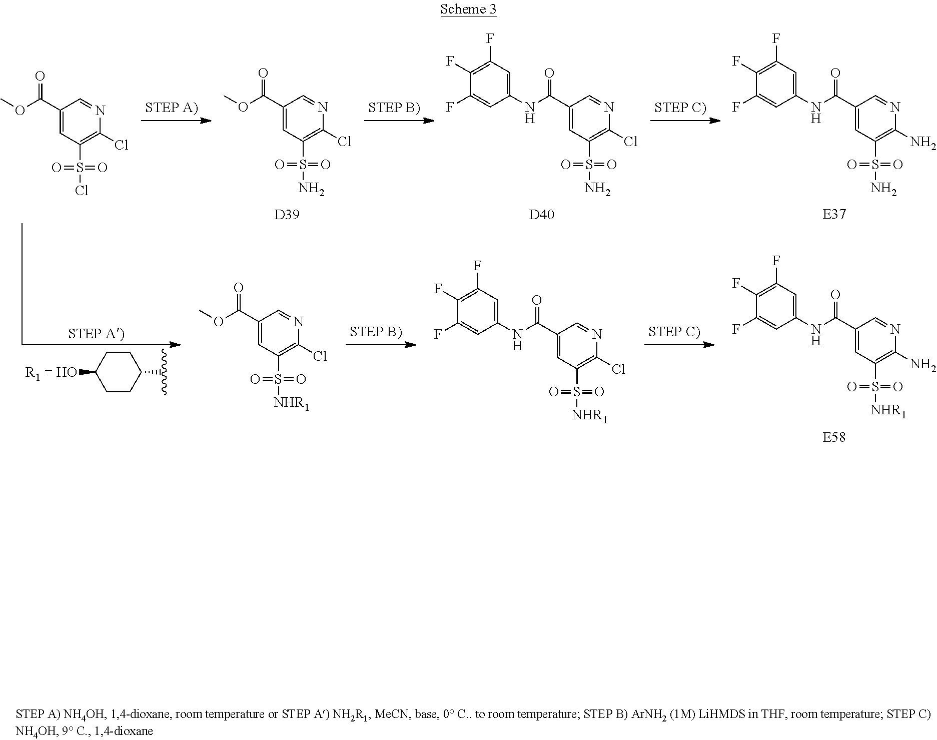

Examples E59, E61 and E62 can be prepared according to Scheme 4:

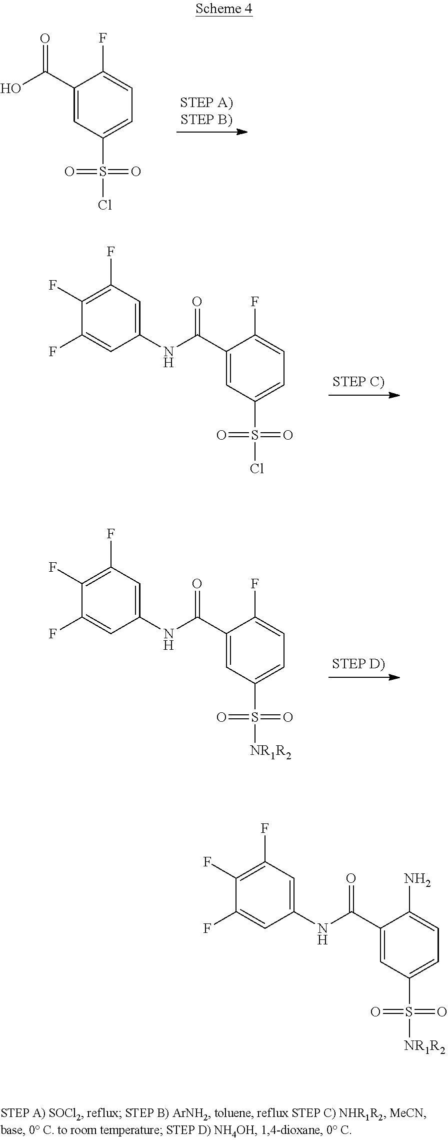

The following Examples illustrate the preparation of certain compounds of Formula (I), (Ia) or salts thereof. The Descriptions 1 to 40 illustrate the preparation of intermediates used to make compounds of the invention or salts thereof. Examples 1 to 71 illustrate the present invention.

In the procedures that follow, for the starting material reference is typically provided to a procedure in the Descriptions or in the Examples. This is provided merely for assistance to the skilled chemist. The starting material may not necessarily have been prepared from the batch of the Description or the Example referred to.

### EXAMPLES

### Description 1: 3-(chlorosulfonyl)-4-fluorobenzoyl chloride (D1)

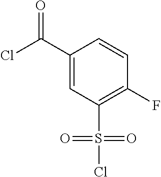 [SMILES: O=C(Cl)c1ccc(F)c(S(=O)(=O)Cl)c1]

3-Chlorosulfonyl-4-fluoro-benzoic acid (Fluorochem, cat no 037319) (14.2 g, 59.51 mmol) was added to thionyl chloride (80.39 mL, 1106.9 mmol) in a single portion. The resulting yellowish solution was heated to reflux for 4 hrs, giving a slurry. Solvent was removed in vacuo by co-evaporation with toluene, giving the title compound D1 (15.4 g, 59.91 mmol) as a brown oil.

### Description 2: 2-chloro-5-(chlorosulfonyl)-4-fluorobenzoyl chloride (D2)

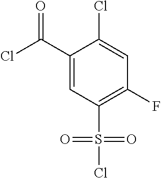 [SMILES: O=C(Cl)c1cc(S(=O)(=O)Cl)c(F)cc1Cl]

Similarly prepared according to procedure described for the preparation of compound D1, starting from 2-Chloro-5-chlorosulfonyl-4-fluoro-benzoic acid (Enamine, cat. no EN300-01843).

### Description 3: 2-bromo-5-(chlorosulfonyl)-4-fluorobenzoyl chloride (D3)

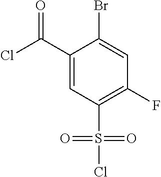 [SMILES: O=C(Cl)c1cc(S(=O)(=O)Cl)c(F)cc1Br]

Similarly prepared according to procedure described for the preparation of D1, starting from 2-Bromo-5-(chlorosulfonyl)-4-fluorobenzoic acid (Enamine, cat. no EN300-52736).

### Description 4: 5-(chlorosulfonyl)-2,4-difluorobenzoyl chloride (D4)

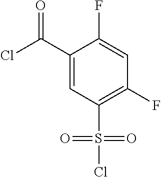 [SMILES: O=C(Cl)c1cc(S(=O)(=O)Cl)c(F)cc1F]

Similarly prepared according to procedure described for the preparation of D1, starting from 5-(Chlorosulfonyl)-2,4-difluorobenzoic acid (Enamine, cat. no EN300-59276).

### Description 5: 3-(chlorosulfonyl)-4,5-difluorobenzoyl chloride (D5)

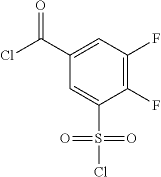 [SMILES: O=C(Cl)c1cc(F)c(F)c(S(=O)(=O)Cl)c1]

Similarly prepared according to the procedure described for the preparation of D1, starting from 3-(Chlorosulfonyl)-4,5-difluorobenzoic acid (Enamine, cat. no EN300-107773).

### Description 6: 5-(chlorosulfonyl)-4-fluoro-2-methylbenzoyl chloride (D6)

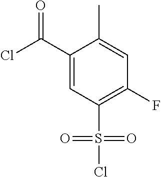

Similarly prepared according to procedure described for the preparation of D1, starting from 5-(chlorosulfonyl)-4-fluoro-2-methylbenzoic acid (Enamine, cat. no EN300-114063).

### Description 7: 2-fluoro-5-((3,4,5-trifluorophenyl)carbamoyl)benzenesulfonyl chloride (D7)

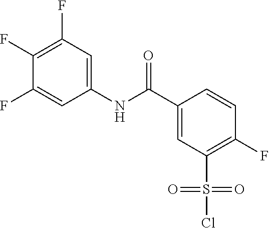 [SMILES: O=C(Nc1cc(F)c(F)c(F)c1)c1ccc(F)c(S(=O)(=O)Cl)c1]

D1 (15.4 g, 59.91 mmol) was dissolved in toluene (140 mL), heated to reflux and then a solution of 3,4,5-trifluoroaniline (8.81 g, 59.91 mmol) in toluene (50 mL) was added dropwise over 10 min. A suspension was formed and refluxed for 1 h. The reaction was cooled to room temperature, filtered and the cake obtained washed with a small amount of toluene, giving the title compound D7 (14.5 g, 39.43 mmol) as off-white solid. ¹H NMR (300 MHz, DMSO-d₆) δ ppm 7.32 (t, J 9.03 Hz, 1H) 7.68-7.81 (m, 2H) 7.90-8.03 (m, 1H) 8.30 (dd, J=6.83, 2.43 Hz, 1H) 10.71 (s, 1H). Method 1: Rt=2.43 min, m/z=368.32 (M+H)⁺.

### Description 8: 5-((3,4-difluorophenyl)carbamoyl)-2-fluorobenzenesulfonyl chloride (D8)

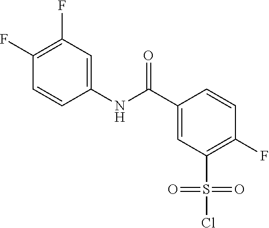 [SMILES: O=C(Nc1ccc(F)c(F)c1)c1ccc(F)c(S(=O)(=O)Cl)c1]

To a solution of D1 (1.8 g, 7.0 mmol) in dry toluene (13.5 mL) at 90° C., triethylamine (1 mL, 7.175 mmol) and a solution of 3,4-difluoroaniline (0.904 g, 7.0 mmol) in dry toluene (3.5 mL) were added dropwise. Reaction mixture was stirred at reflux for 30 min, then was allowed to cool at room temperature and diluted with dichloromethane. Organic layer was washed with brine, dried over Na₂SO₄, filtered and evaporated under reduced pressure to give a beige solid (2.39 g) as crude product was suspended in DCM (5 mL) and sonicated. The resulting solid was filtered, washed with DCM and dried at vacuum pump to afford the title compound D8 as white powder (1.73 g). ¹H NMR (300 MHz, DMSO-d₆) δ ppm 7.26-7.48 (m, 3H) 7.52-7.61 (m, 1H) 7.91-7.99 (m, 2H) 8.30 (dd, J=6.88, 2.38 Hz, 1H) 10.59 (s, 1H). Method 1: Rt=2.43 min, m/z=350.02 (M+H)+.

### Description 9: 5-[(3-chloro-4-fluorophenyl)carbamoyl]-2-fluorobenzene-1-sulfonyl chloride (D9)

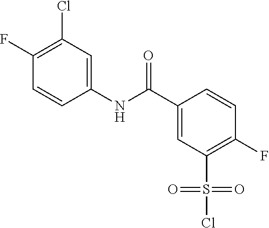 [SMILES: O=C(Nc1ccc(F)c(Cl)c1)c1ccc(F)c(S(=O)(=O)Cl)c1]

Similarly prepared according to procedure described for the preparation of D8 starting from 3-(chlorosulfonyl)-4-fluorobenzoyl chloride D1 and using 3-Chloro-4-fluoroaniline instead of 3,4-Difluoroaniline, to give the title compound D9. Method 1: Rt=2.43 min, m/z=366.01 (M+H)+.

### Description 10: 2-fluoro-5-((4-fluoro-3-(trifluoromethyl)phenyl)carbamoyl)benzenesulfonyl chloride (D10)

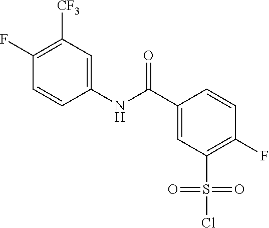 [SMILES: O=C(Nc1ccc(F)c(C(F)(F)F)c1)c1ccc(F)c(S(=O)(=O)Cl)c1]

Similarly prepared according to procedure described for the preparation of D7, using as starting material D1 (500 mg, 1.94 mmol) and 4-fluoro-3-(trifluoromethyl)aniline (0.25 mL, 1.94 mmol) instead of 3,4,5-trifluoroaniline. The reaction mixture was concentrated in vacuo to give to give the title compound D10 (805 mg) as beige solid, that was used in the next synthetic step directly.

### Description 11: 5-((3-cyano-4-fluorophenyl)carbamoyl)-2-fluorobenzenesulfonyl chloride (D11)

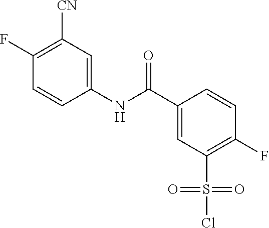 [SMILES: N#Cc1cc(NC(=O)c2ccc(F)c(S(=O)(=O)Cl)c2)ccc1F]

Similarly prepared according to procedure described for the preparation of D7, using as starting materials D1 and 5-Amino-2-fluorobenzonitrile instead of 3,4,5-trifluoroaniline.

### Description 12: 5-((3-(difluoromethyl)-4-fluorophenyl)carbamoyl)-2-fluorobenzenesulfonyl chloride (D12)

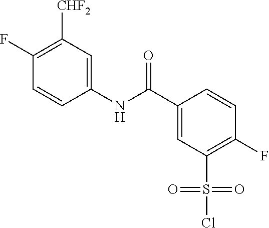 [SMILES: O=C(Nc1ccc(F)c(C(F)F)c1)c1ccc(F)c(S(=O)(=O)Cl)c1]

Similarly prepared according to procedure described for the preparation of D7, using as starting materials D1 and 3-(Difluoromethyl)-4-fluoroaniline instead of 3,4,5-trifluoroaniline.

### Description 13: 5-((6-chloropyridin-3-yl)carbamoyl)-2-fluorobenzenesulfonyl chloride (D13)

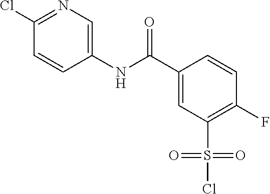 [SMILES: O=C(Nc1ccc(Cl)nc1)c1ccc(F)c(S(=O)(=O)Cl)c1]

D1 (307 mg, 1.19 mmol) was dissolved in Toluene (2.7 mL) and heated to 90° C. A suspension of 6-chloropyridin-3-amine (153.5 mg, 1.19 mmol) in Toluene (1.2 mL) was slowly added and the reaction mixture was refluxed at 110° C. for 20 min. DIPEA (0.31 mL, 1.79 mmol) was then added, and the reaction was refluxed at 110° C. for another 1 h 50 min. The mixture was concentrated in vacuo to give the crude title compound D13 (702 mg) as brown oil, that was used in the next synthetic step directly. Method 1; Rt=2.17 min, m/z=349.00 (M+H)⁺.

### Description 14: 2-fluoro-4-methyl-5-((3,4,5-trifluorophenyl)carbamoyl)benzenesulfonyl chloride (D14)

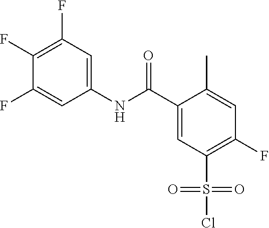 [SMILES: Cc1cc(F)c(S(=O)(=O)Cl)cc1C(=O)Nc1cc(F)c(F)c(F)c1]

Similarly prepared according to procedure described for the preparation of D7, starting from 5-(chlorosulfonyl)-4-fluoro-2-methylbenzoyl chloride D6 instead of D1. Method 1; Rt=2.49 min, m/z=382.11 (M+H)⁺.

### Description 15: 2-fluoro-5-((4-fluoro-3-methylphenyl)carbamoyl)benzenesulfonyl chloride (D15)

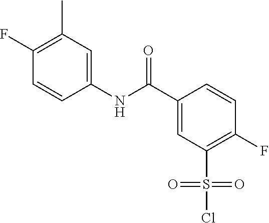 [SMILES: Cc1cc(NC(=O)c2ccc(F)c(S(=O)(=O)Cl)c2)ccc1F]

Similarly prepared according to procedure described for the preparation of D7, using as starting materials D1 and 4-Fluoro-3-methylaniline instead of 3,4,5-trifluoroaniline. Method 1; Rt=2.38 min, m/z=346.17 (M+H)⁺.

### Description 16: 5-((3,5-difluoro-4-methylphenyl)carbamoyl)-2-fluorobenzenesulfonyl chloride (D16)

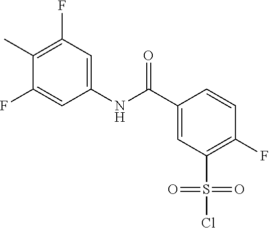 [SMILES: Cc1c(F)cc(NC(=O)c2ccc(F)c(S(=O)(=O)Cl)c2)cc1F]

Similarly prepared according to procedure described for the preparation of D7, using as starting materials D1 and 3,5-Difluoro-4-methylaniline instead of 3,4,5-trifluoroaniline. Method 1; Rt=2.54 min, m/z=364.20 (M+H)⁺.

### Description 17: 2-fluoro-5-((2,3,4-trifluorophenyl)carbamoyl)benzenesulfonyl chloride (D17)

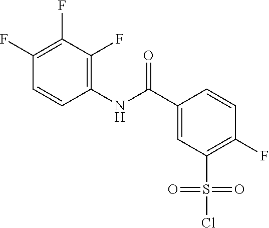 [SMILES: O=C(Nc1ccc(F)c(F)c1F)c1ccc(F)c(S(=O)(=O)Cl)c1]

Similarly prepared according to procedure described for the preparation of D7, using as starting materials D1 and 2,3,4-trifluoroaniline instead of 3,4,5-trifluoroaniline. Method 1; Rt=2.29 min, m/z=368.19 (M+H)⁺.

### Description 18: 2-fluoro-5-((2,4,5-trifluorophenyl)carbamoyl)benzenesulfonyl chloride (D18)

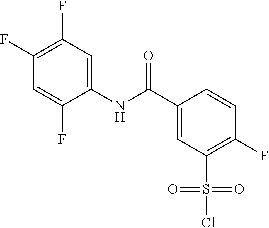 [SMILES: O=C(Nc1cc(F)c(F)cc1F)c1ccc(F)c(S(=O)(=O)Cl)c1]

Similarly prepared according to procedure described for the preparation of D7, using as starting materials D1 and 2,4,5-trifluoroaniline instead of 3,4,5-trifluoroaniline. Method 1; Rt=2.32 min, m/z=368.39 (M+H)⁺.

### Description 19: 2-fluoro-5-((2,4,5-trifluorophenyl)carbamoyl)benzenesulfonyl chloride (D19)

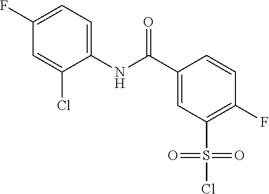 [SMILES: O=C(Nc1ccc(F)cc1Cl)c1ccc(F)c(S(=O)(=O)Cl)c1]

Similarly prepared according to procedure described for the preparation of D7, using as starting materials D1 and 2-Chloro-4-fluoroaniline instead of 3,4,5-trifluoroaniline. Method 1; Rt=2.32 min, m/z=366.03 (M+H)⁺.

### Description 20: 4-fluoro-3-sulfamoyl-N-(3,4,5-trifluorophenyl)benzamide (D20)

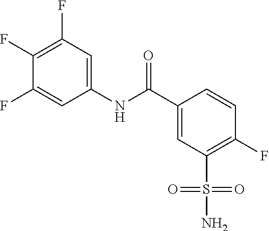 [SMILES: NS(=O)(=O)c1cc(C(=O)Nc2cc(F)c(F)c(F)c2)ccc1F]

D7 (3 g, 8.16 mmol) was dissolved in 1,4-Dioxane (20 mL, 0.23 mol) and added in a single portion to ammonia (10.42 mL, 163.18 mmol) at 0° C. The reaction was stirred 15 min then diluted with water and extracted with 2Me-THF. The organic layer was washed with 6N HCl and evaporated giving a brown solid (2.5 g) that was triturated with DCM giving the title compound D20 (2 g, 5.74 mmol) as off-white solid. Method 1; Rt: 1.85 min. m/z: 349.21 (M+H)⁺.

### Description 21: 2-chloro-4-fluoro-5-sulfamoyl-N-(3,4,5-trifluorophenyl)benzamide (D21)

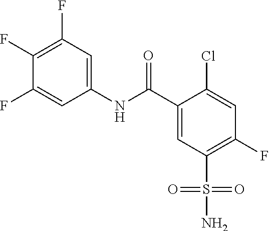 [SMILES: NS(=O)(=O)c1cc(C(=O)Nc2cc(F)c(F)c(F)c2)c(Cl)cc1F]

A 20 mL vial was charged with 3,4,5-Trifluoroaniline (245.4 mg, 1.67 mmol) then a solution of D2 (221.04 mg, 0.76 mmol) in Toluene (5 mL) was added in a single portion. The vial was sealed and the reaction mixture stirred for 10 min at room temperature, resulting in a white slurry. The reaction was diluted with toluene (3 mL) and heated by microwave irradiation at 70° C. for 20 min. The reaction slurry was cooled to room temperature and poured into a flask containing acqu. NH₄OH (approx. 30 mL) and stirred vigourously at room temperature overnight. The resulting white slurry, was diluted with DCM, treated with ice and acidified with 6N HCl until pH=1. The organic layer was diluted with EtOAc, washed with 6N HCl two times and brine, dried over MgSO₄ (dry), filtered and finally evaporated giving the title compound D21 (250 mg, 0.65 mmol) as white solid. ¹H NMR (300 MHz, DMSO-d₆) δ ppm 7.13-7.30 (m, 1H) 7.53-7.66 (m, 2H) 7.89-8.02 (m, 3H) 11.05 (s, 1H). Method 9; Rt: 2.02 min. m/z: 383.05 (M+H)⁺.

### Description 22: 2-bromo-4-fluoro-5-sulfamoyl-N-(3,4,5-trifluorophenyl)benzamide (D22)

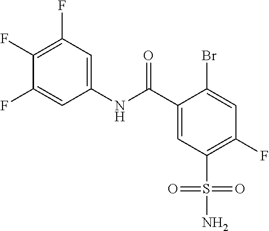 [SMILES: NS(=O)(=O)c1cc(C(=O)Nc2cc(F)c(F)c(F)c2)c(Br)cc1F]

A 20 mL vial was charged with D3 (500 mg, 1.49 mmol), 3,4,5-Trifluoroaniline (437.85 mg, 2.98 mmol) and Toluene (4 mL). The vial was sealed and stirred at room temperature giving after 10 min a white suspension. The reaction was diluted with toluene (3 mL) and the reaction mixture was heated by microwave irradiation at 100° C. for 15 min, diluted with toluene (5 mL) and poured into 37% NH₄OH (20 mL). MeCN (2 mL) was added and the resulting mixture stirred for 4 hrs at room temperature.

The reaction mixture was diluted with EtOAc and extracted with EtOAc; the organic layer was dried over MgSO₄ (dry), filtered and evaporated giving the title compound D22 (700 mg, purity 75%). This intermediate was used in the next step without any purification. Method 1; Rt: 1.93 min. m/z: 427.16 (M+H)⁺.

### Description 23: N-(3-chloro-4-fluorophenyl)-4-fluoro-3-sulfamoylbenzamide (D23)

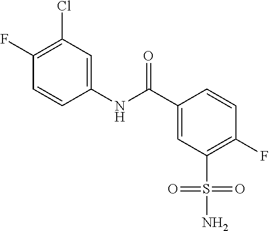 [SMILES: NS(=O)(=O)c1cc(C(=O)Nc2ccc(F)c(Cl)c2)ccc1F]

D9 (200 mg, 0.55 mmol) was suspended in MeCN (2 mL), cooled to 0° C. with ice bath, then treated with a single portion of aqueous ammonia (2.18 mL, 10.92 mmol). The reaction was stirred 5 min at 0° C. giving a yellowish solution. Solvent was removed in vacuo and the residue was diluted with water and extracted with EtOAc. Solvent was removed in vacuo affording the title compound D23 (200 mg, 0.55 mmol). Method 1; Rt: 1.88 min. m/z: 346.98 (M+H)⁺.

### Description 24: 4-fluoro-3-(methylsulfamoyl)-N-(3,4,5-trifluorophenyl)benzamide (D24)

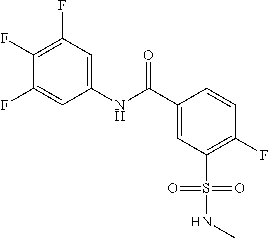 [SMILES: CNS(=O)(=O)c1cc(C(=O)Nc2cc(F)c(F)c(F)c2)ccc1F]

To a solution of D7 (150 mg, 0.41 mmol) in MeCN (3.55 mL) cooled to 0° C., DIPEA (217.17 uL, 1.22 mmol) and methanamine (27.54 mg, 0.41 mmol) were added and the reaction mixture was stirred at room temperature for 1.5 h. Water (5 mL) was added and the reaction mixture was stirred for 15 min. DCM (5 mL) was added and the stirring was continued for further 15 min. The solution was filtered on phase separator and the organic layer was concentrated under vacuo to give the crude title compound D24 (151 mg) which was used without purification. Method 1; Rt=2.01 min, m/z=362.79 (M+H)⁺.

### Description 25: (R)-4-fluoro-N-(3,4,5-trifluorophenyl)-3-(N-(1,1,1-trifluoropropan-2-yl)sulfamoyl)benzamide (D25)

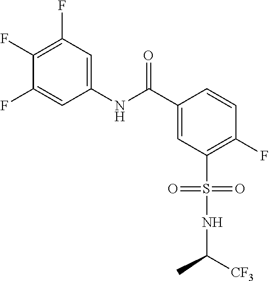 [SMILES: C[C@@H](NS(=O)(=O)c1cc(C(=O)Nc2cc(F)c(F)c(F)c2)ccc1F)C(F)(F)F]

D7 (100 mg, 0.27 mmol) was suspended in MeCN (2 mL), DIPEA (0.24 mL, 1.36 mmol) and (2R)-1,1,1-trifluoro-2-propanamine hydrochloride (81.34 mg, 0.54 mmol) were added, and the resulting solution was stirred at RT for 3 h. The reaction was diluted with EtOAc and washed twice with 3N HCl solution. Organic layer was dried over Na₂SO₄, filtered and concentrated under vacuo, to obtain the crude title compound D25 (88 mg) that was used in the next step without further purification. Method 1; Rt=2.32 min, m/z=445.06 (M+H)⁺.

### Description 26: (S)-4-fluoro-N-(3,4,5-trifluorophenyl)-3-(N-(1,1,1-trifluoropropan-2-yl)sulfamoyl)benzamide (D26)

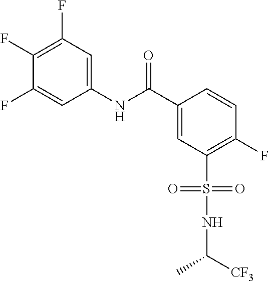 [SMILES: C[C@H](NS(=O)(=O)c1cc(C(=O)Nc2cc(F)c(F)c(F)c2)ccc1F)C(F)(F)F]

Similarly prepared according to procedure described for the preparation of compound D25 using (2S)-1,1,1-Trifluoro-2-propanamine hydrochloride instead of (2R)-1,1,1-Trifluoro-2-propanamine hydrochloride. Method 1; Rt=2.32 min, m/z=445.13 (M+H)⁺.

### Description 27: 3-(N-cyclopropylsulfamoyl)-4-fluoro-N-(3,4,5-trifluorophenyl)benzamide (D27)

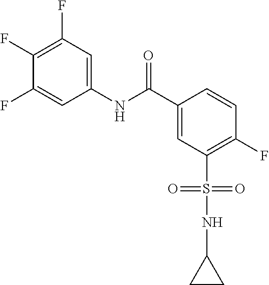 [SMILES: O=C(Nc1cc(F)c(F)c(F)c1)c1ccc(F)c(S(=O)(=O)NC2CC2)c1]

A suspension of cyclopropanamine hydrochloride (113.06 uL, 1.63 mmol) in MeCN (1 mL) was treated at 0° C. with DIPEA and then with D7 (150 mg, 0.41 mmol). The resulting yellow solution was stirred at room temperature. Solvent was removed in vacuo, the residue was dissolved in 1/1 EtOAc/2-Methyl-THF and poured into a separating funnel. The organic layer was washed with 6N HCl, NaHCO₃ aq.sat. solution and brine, dried over Na₂SO₄, filtered and finally evaporated giving the title compound D27 (125 mg) as white solid. Method 1; Rt: 2.16 min. m/z: 389.13 (M+H)⁺.

### Description 28: trans-4-fluoro-3-(N-(4-hydroxycyclohexyl)sulfamoyl)-N-(3,4,5-trifluorophenyl)benzamide (D28)

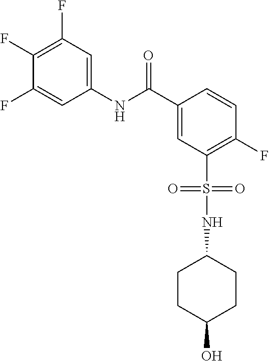 [SMILES: O=C(Nc1cc(F)c(F)c(F)c1)c1ccc(F)c(S(=O)(=O)N[C@H]2CC[C@H](O)CC2)c1]

A suspension of trans-4-aminocyclohexan-1-ol hydrochloride (80 mg, 0.53 mmol) in MeCN (1 mL) was treated at 0° C. with triethylamine (197.92 uL, 1.43 mmol) and then with D7 (150 mg, 0.41 mmol) The resulting solution become a white suspension and it was stirred at room temperature. Solvent was removed in vacuo, the residue was dissolved in 1/1 EtOAc/2-Methyl-THF and poured into a separating funnel. The organic layer was washed with 6N HCl, NaHCO₃ aq.sat.solution and brine, dried over Na₂SO₄, filtered and finally evaporated giving the title compound D28 (100 mg) as white solid. Method 1; Rt: 1.90 min. m/z: 447.16 (M+H)⁺.

### Description 29: cis-4-fluoro-3-(N-(4-hydroxycyclohexyl)sulfamoyl)-N-(3,4,5-trifluorophenyl)benzamide (D29)

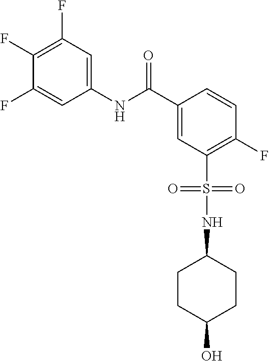 [SMILES: O=C(Nc1cc(F)c(F)c(F)c1)c1ccc(F)c(S(=O)(=O)N[C@H]2CC[C@@H](O)CC2)c1]

Similarly prepared according to procedure described for the preparation of D25 using cis-4-Amino-cyclohexanol hydrochloride instead of (2R)-1,1,1-trifluoro-2-propanamine hydrochloride. Method 1; Rt=2.00 min, m/z=447.22 (M+H)⁺.

### Description 30: trans-4-fluoro-5-(N-((4-hydroxycyclohexyl)sulfamoyl)-2-methyl-N-(3,4,5-trifluorophenyl)benzamide (D30)

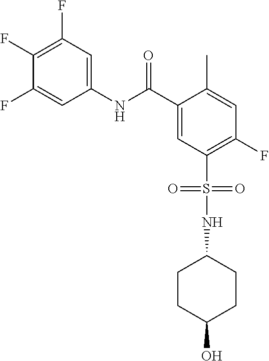 [SMILES: Cc1cc(F)c(S(=O)(=O)N[C@H]2CC[C@H](O)CC2)cc1C(=O)Nc1cc(F)c(F)c(F)c1]

D14 (100 mg, 0.26 mmol) was dissolved in MeCN (1.3 mL) and cooled to 0° C. Triethylamine (0.13 mL, 0.92 mmol) and trans-4-Aminocyclohexanol hydrochloride (43.7 mg, 0.29 mmol) were added, and the reaction mixture was stirred at RT for 1 h. EtOAc and few drops of MeOH were added, and the mixture was washed with 5% citric acid solution and sat. NaHCO₃ solution. Organic layer was dried over Na₂SO₄, filtered and concentrated under vacuo, to obtain crude D30 (122 mg) as white solid. Method 1; Rt: 2.03 min. m/z: 461.34 (M+H)⁺.

### Description 31: tert-butyl 4-((2-fluoro-5-((3,4,5-trifluorophenyl)carbamoyl)phenyl)sulfonamido)piperidine-1-carboxylate (D31)

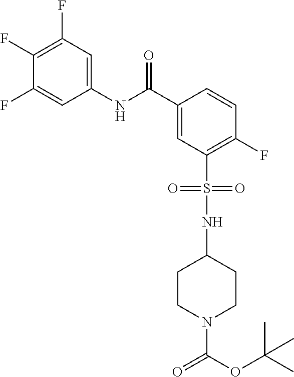 [SMILES: CC(C)(C)OC(=O)N1CCC(NS(=O)(=O)c2cc(C(=O)Nc3cc(F)c(F)c(F)c3)ccc2F)CC1]

Tert-butyl 4-aminopiperidine-1-carboxylate (245.11 mg, 1.22 mmol) was suspended in MeCN (2 mL), cooled to 0° C. and treated with a single portion of D7 (150 mg, 0.41 mmol). The resulting white suspension was stirred at room temperature for 1 h. Solvent was removed in vacuo, the residue was dissolved in 1/1 EtOAc/2-Methyl-THF and poured into a separating funnel. The organic layer was washed with 6N HCl, NaHCO₃ sat. solution and brine, dried over Na₂SO₄, filtered and finally evaporated giving the title compound D31 (140 mg) as white solid. Method 1; Rt: 2.37 min. m/z: 432.23 (M-Boc)⁺.

### Description 32: 4-fluoro-3-(N-(pyridin-4-yl)sulfamoyl)-N-(3,4,5-trifluorophenyl)benzamide (D32)

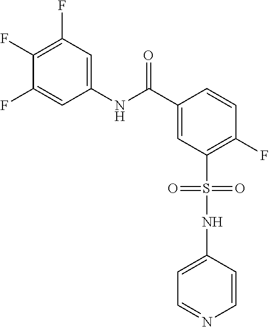 [SMILES: O=C(Nc1cc(F)c(F)c(F)c1)c1ccc(F)c(S(=O)(=O)Nc2ccncc2)c1]

D7 (50 mg, 0.14 mmol) was suspended in MeCN (1 mL), treated with triethylamine (56.55 uL, 0.41 mmol) and pyridin-4-amine (40 mg, 0.43 mmol) resulting in a yellowish solution. The reaction was diluted with 2-Methyl-THF and EtOAc and acidified with 1N HCl to pH=1 (by paper), poured into a separating funnel and the aqueous layer separated. The aqueous layer was basified with NaHCO₃ and extracted with EtOAc; the combined organic extracts were evaporated, giving a residue containing the crude product (60 mg). The crude product was suspended in DCM and filtered giving the title compound D32 as yellowish solid. Method 1; Rt: 1.57 min. m/z: 426.15 (M+H)⁺.

### Description D33: 4-fluoro-3-(N-(oxetan-3-yl)sulfamoyl)-N-(3,4,5-trifluorophenyl)benzamide (D33)

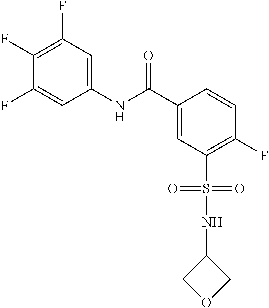 [SMILES: O=C(Nc1cc(F)c(F)c(F)c1)c1ccc(F)c(S(=O)(=O)NC2COC2)c1]

A solution of D7 (300 mg, 0.82 mmol) in MeCN (2 mL) was added to 3-oxetanamine (0.06 mL, 0.82 mmol) in a single portion at room temperature. The resulting white suspension was stirred at the same temperature for 1 hr. Solvent was removed in vacuo, the residue dissolved in DCM, washed with 5% citric acid aqueous solution, dried over Na₂SO₄, filtered and finally evaporated giving the title compound D33 (300 mg, 0.74 mmol) as white solid. Method 1; Rt: 1.99 min. m/z: 405.14 (M+H)⁺.

### Description 34: tert-butyl (S)-3-((2-fluoro-5-((3,4,5-trifluorophenyl)carbamoyl)phenyl)sulfonamido)pyrrolidine-1-carboxylate (D34)

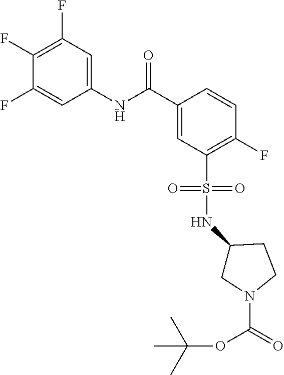 [SMILES: CC(C)(C)OC(=O)N1CC[C@H](NS(=O)(=O)c2cc(C(=O)Nc3cc(F)c(F)c(F)c3)ccc2F)C1]

A solution of D7 (200 mg, 0.54 mmol) in MeCN (2 mL) was added to tert-butyl (S)-3-aminopyrrolidine-1-carboxylate (202.61 mg, 1.09 mmol in a single portion at room temperature. The resulting white suspension was stirred at room temperature for 1 hr. Solvent was removed in vacuo, the residue dissolved in DCM, washed with 5% citric acid aqueous solution, dried over Na₂SO₄, filtered and finally evaporated giving the title compound D34 (280 mg, 0.54 mmol) as oil. Method 1; Rt: 2.35 min. m/z: 518.22 (M+H)⁺.

### Description 35: 4-fluoro-3-(N-(3-hydroxycyclobutyl)sulfamoyl)-N-(3,4,5-trifluorophenyl)benzamide (D35)

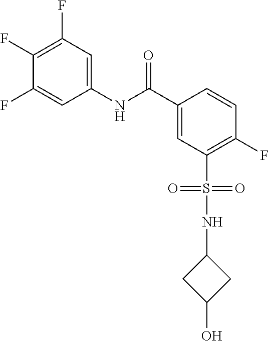 [SMILES: O=C(Nc1cc(F)c(F)c(F)c1)c1ccc(F)c(S(=O)(=O)NC2CC(O)C2)c1]

A solution of 3-aminocyclobutanol hydrochloride (134.44 mg, 1.09 mmol) in MeCN (2 mL) was treated with a single portion of DIPEA (229.33 uL, 1.63 mmol) and then with D7 (200 mg, 0.54 mmol). The resulting solution was stirred at room temperature for 1 h. Solvent was removed in vacuo, the residue dissolved in DCM, washed with 5% citric acid aqueous solution, dried over Na₂SO₄, filtered and finally evaporated giving the title compound D35 (220 mg, 0.526 mmol) as oil. Method 1; Rt: 1.89 min. m/z: 419.19 (M+H)⁺.

### Description 36: 4-fluoro-3-(N-((1R,3R)-3-hydroxycyclopentyl)sulfamoyl)-N-(3,4,5-trifluorophenyl)benzamide (D36)

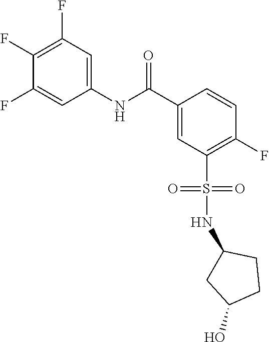 [SMILES: O=C(Nc1cc(F)c(F)c(F)c1)c1ccc(F)c(S(=O)(=O)N[C@H]2CC[C@H](O)C2)c1]

A suspension of (1S,3S)-3-Aminocyclopentan-1-ol hydrochloride 56.14 mg, 0.410 mmol (cod. I-9981, Advanced ChemBlocks) in MeCN (1.5 mL) was treated with N-ethyl-N-isopropylpropan-2-amine (198.95 uL, 1.14 mmol) and stirred at room temperature for 15 min. The reaction solution was cooled with ice bath and treated with a single portion of D7 (100. mg, 0.270 mmol) and stirred for 5 min at 0° C. and at room temperature for 10 min giving a yellow solution. Solvent was removed in vacuo, the residue was partitioned in DCM and citric acid (5% acq. solution). The organic layer was dried over Na₂SO₄ (anh.), filtered and evaporated, giving D36 (105 mg).

### Description 37: methyl 4-fluoro-3-methyl-5-sulfamoylbenzoate (D37)

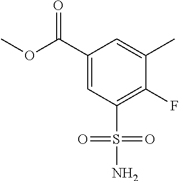 [SMILES: COC(=O)c1cc(C)c(F)c(S(N)(=O)=O)c1]

Similarly prepared according to procedure described for the preparation of the preparation of D20 using as starting material methyl 3-(chlorosulfonyl)-4-fluoro-5-methylbenzoate (Enamine, cat. n° EN300-266824). Method 1; Rt=1.39 min, m/z=248.14 (M+H)⁺.

### Description 38: Synthesis of Compound: 4-fluoro-3-methyl-5-sulfamoyl-N-(3,4,5-trifluorophenyl)benzamide (D38)

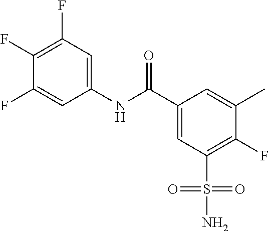 [SMILES: Cc1cc(C(=O)Nc2cc(F)c(F)c(F)c2)cc(S(N)(=O)=O)c1F]

To a solution of methyl 4-fluoro-3-methyl-5-sulfamoylbenzoate D37 (150 mg, 0.610 mmol) and 3,4,5-Trifluoroaniline (116 mg 0.79 mmol) in dry THF (3 mL), lithium bis(trimethylsilyl)amide 1M in THF (3.52 mL, 3.52 mmol) was added dropwise. The reaction mixture was stirred at RT for 3 h, then more 3,4,5-Trifluoroaniline (50 mg, 0.340 mmol) and lithium bis(trimethylsilyl)amide 1M in THF (1 mL, 1 mmol) were added, and the reaction mixture was stirred at RT overnight. The reaction was quenched with sat. NH₄Cl solution, and EtOAc was added. Organic layer was washed with brine, then was dried over Na₂SO₄, filtered and concentrated under vacuo. The residue was triturated with PE, to obtain the title compound D38 (218 mg) as light-brown solid, that was used without further purification. Method 1; Rt=1.97 min, m/z=363.12 (M+H)⁺. ¹H NMR (300 MHz, DMSO-d₆) δ ppm 2.40 (d, J=2.11 Hz, 3H) 7.63-8.01 (m, 4H) 8.12-8.19 (m, 1H) 8.19-8.25 (m, 1H) 10.79 (br s, 1H).

### Description 39: methyl 6-chloro-5-sulfamoylnicotinate (D39)

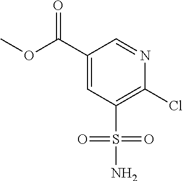

Methyl 6-chloro-5-(chlorosulfonyl)nicotinate (Enamine, EN300-41733; 525 mg, 1.94 mmol) was dissolved in 1,4-dioxane (2 mL), 33% aqueous ammonia (2.29 mL, 19.44 mmol) was added and the reaction mixture was stirred 20 min at RT. The reaction was diluted with EtOAc and water, the phases were separated, the organic layer was dried over Na₂SO₄, filtered and concentrated under vacuo to obtain crude title compound D39 (285 mg), that was used without further purification. Method 1; Rt=1.20 min, m/z=250.91 (M+H)⁺.

### Description D40: 6-chloro-5-sulfamoyl-N-(3,4,5-trifluorophenyl)nicotinamide (D40)

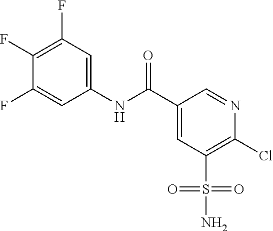 [SMILES: NS(=O)(=O)c1cc(C(=O)Nc2cc(F)c(F)c(F)c2)cnc1Cl]

To a solution of crude compound D39 (144 mg, 0,570 mmol) and 3,4,5-trifluoroaniline (109.9 mg, 0.750 mmol) in dry THF (2.5 mL), lithium bis(trimethylsilyl)amide 1M in THF (3.33 mL, 3.33 mmol) was added dropwise. The reaction mixture was stirred at RT for 1 h. The reaction was quenched with sat. NH₄Cl solution, and EtOAc was added. The organic layer was dried over Na₂SO₄, filtered and concentrated under vacuo. The resulting crude product was purified by preparative HPLC (H₂O/CH₃CN+1% TFA) to obtain, after lyophilization, the title compound D40 (40 mg) as light-yellow solid. Method 3; Rt: 3.17 min, m/z: 366.10 (M+H)⁺. ¹H NMR (300 MHz, DMSO-d₆) δ ppm 7.61-7.82 (m, 2H) 8.01 (s, 2H) 8.81 (d, J=2.29 Hz, 1H) 9.13 (d, J=2.29 Hz, 1H) 11.00 (s, 1H).

### Example 1: 4-(methylamino)-3-sulfamoyl-N-(3,4,5-trifluorophenyl)benzamide (E1)

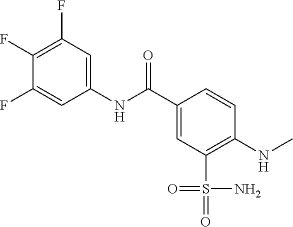 [SMILES: CNc1ccc(C(=O)Nc2cc(F)c(F)c(F)c2)cc1S(N)(=O)=O]

D20 (58. mg, 0.170 mmol) was dissolved in DMSO (1 mL, 0.014 mol) and treated with methanamine (77.59 mg, 2.5 mmol) and Triethylamine (346.27 uL, 2.5 mmol). The reaction solution was stirred overnight at room temperature. A second portion of methanamine (77.59 mg, 2.5 mmol) was added followed by triethylamine (346.27 uL, 2.5 mmol). After 5 hrs at room temperature MeCN (200 uL) were added and the reaction was stirred overnight at room temperature. The reaction was diluted with water and DCM, poured into a separating funnel and the organic layer washed twice with water and brine, dried over MgSO₄ (dry), filtered and finally evaporated yielding a yellowish solid (72 mg). DCM (1 mL) was added and the resulting white suspension was filtered yielding the title compound E1 as white solid (33 mg). Method 3; Rt: 3.27 min. MS(ES+) m/z: 360.15 (M+H)⁺. ¹H NMR (300 MHz, DMSO-d₆) δ ppm 2.93 (d, J=4.86 Hz, 3H) 6.30-6.47 (m, 1H) 6.86 (d, J=8.89 Hz, 1H) 7.43 (br s, 2H) 7.64-7.86 (m, 2H) 8.06 (dd, J=8.76, 2.06 Hz, 1H) 8.33 (d, J=2.20 Hz, 1H) 10.41 (br s, 1H).

### Example 2: 4-amino-3-(N-methylsulfamoyl)-N-(3,4,5-trifluorophenyl)benzamide (E2)

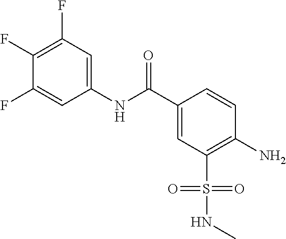 [SMILES: CNS(=O)(=O)c1cc(C(=O)Nc2cc(F)c(F)c(F)c2)ccc1N]

To a solution of crude D24 (151 mg), in 1,4-dioxane (1.04 mL), aqueous ammonia (517 uL, 13.27 mmol) was added and the reaction mixture was stirred in a sealed vial at 90° C. for 2 h, and overnight at room temperature. The solvent was evaporated under reduced pressure. The crude product (40.06 mg) was purified by preparative HPLC (H₂O/CH₃CN+1% TFA) to afford, after lyophilization, the title compound E2 (17.25 mg) as white powder. The remaining crude title compound was used without purification. Method 3; Rt: 3.23 min. m/z: 360.15 (M+H)⁺. ¹H NMR (300 MHz, DMSO-d₆) δ ppm 2.44 (d, J=5.10 Hz, 3H) 6.57 (br s, 2H) 6.95 (d, J=8.70 Hz, 1H) 7.50-7.59 (m, 1H) 7.68-7.81 (m, 2H) 0.00 (dd, J=9.80, 2.10 Hz, 1H) 0.00 (d, J=2.10 Hz, 1H) 10.36 (br s, 1H).

### Example 3: 4-amino-3-sulfamoyl-N-(3,4,5-trifluorophenyl)benzamide (E3)

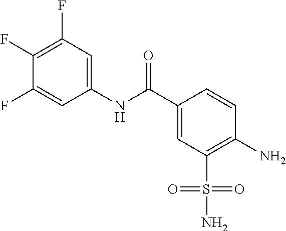 [SMILES: Nc1ccc(C(=O)Nc2cc(F)c(F)c(F)c2)cc1S(N)(=O)=O]

A pressure vessel was charged with D7 (1.2 g, 3.26 mmol), 1,4-dioxane (6 mL) and 33% aqueous ammonia (4 mL, 34 mmol). The pressure vessel was sealed and the reaction mixture was stirred 5 min at RT, then heated at 95° C. for 8 h. The reaction was diluted with EtOAc and water, organic layer was dried over Na₂SO₄, filtered and concentrated under vacuo. The resulting crude product was purified by preparative HPLC (H₂O/CH₃CN+1% TFA) to obtain, after lyophilization, the title compound E3 (840 mg) as white solid. Method 3; Rt=3.01 min, m/z=346.10 (M+H)⁺. ¹H NMR (300 MHz, DMSO-d₆) δ ppm 6.49 (br s, 2H) 6.88 (d, J=8.71 Hz, 1H) 7.37 (s, 2H) 7.62-7.81 (m, 2H) 7.88 (dd, J=8.80, 2.20 Hz, 1H) 8.26 (d, J=2.11 Hz, 1H) 10.33 (s, 1H).

### Example 4: 4-amino-N-(3,4-difluorophenyl)-3-sulfamoylbenzamide (E4)

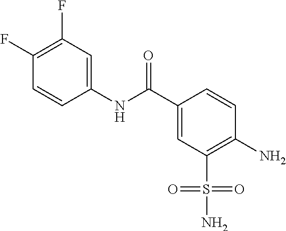 [SMILES: Nc1ccc(C(=O)Nc2ccc(F)c(F)c2)cc1S(N)(=O)=O]

Similarly prepared according to the procedure described for the preparation of E3, starting from D8. Method 3; Rt=2.79 min, m/z=328.20 (M+H)⁺. ¹H NMR (300 MHz, DMSO-d₆) δ ppm 6.49 (br s, 2H) 6.88 (d, J=8.71 Hz, 1H) 7.37 (s, 2H) 7.62-7.81 (m, 2H) 7.88 (dd, J=8.80, 2.20 Hz, 1H) 8.26 (d, J=2.11 Hz, 1H) 10.33 (s, 1H).

### Example 5: 4-amino-2-chloro-5-sulfamoyl-N-(3,4,5-trifluorophenyl)benzamide (E5)

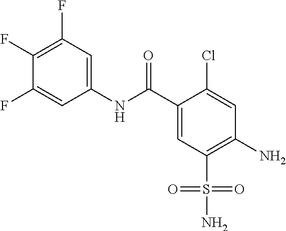 [SMILES: Nc1cc(Cl)c(C(=O)Nc2cc(F)c(F)c(F)c2)cc1S(N)(=O)=O]

D21 (250 mg, 0.650 mmol) was dissolved in 1,4-Dioxane (0.5 mL), treated with ammonia (2. mL, 117.44 mmol) and heated at 90° C. in a closed vial for 12 hrs. The yellowish reaction solution was poured, diluted with EtOAc (approx. 20 mL) and water. The organic layer was washed with brine and 0.2N HCl (2 mL) then evaporated, giving a residue (220 mg). One amount (20 mg) was purified by preparative HPLC (H₂O/CH₃CN+1% TFA) yielding the title compound E5 (11 mg) as white powder. Method 3; Rt: 3.21 min. m/z: 380.15 (M+H)⁺. ¹H NMR (300 MHz, DMSO-d₆) δ ppm 6.46 (br s, 2H) 6.96 (s, 1H) 7.46 (br s, 2H) 7.53-7.70 (m, 2H) 7.76 (br s, 1H) 10.69 (br s, 1H)

### Example 6: 4-amino-2-bromo-5-sulfamoyl-N-(3,4,5-trifluorophenyl)benzamide (E6)

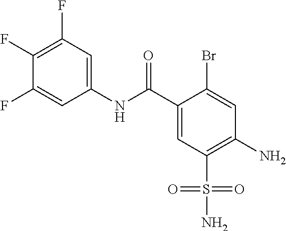 [SMILES: Nc1cc(Br)c(C(=O)Nc2cc(F)c(F)c(F)c2)cc1S(N)(=O)=O]

Similarly prepared according to procedure described for E5, starting from D222-bromo-4-fluoro-5-sulfamoyl-N-(3,4,5-trifluorophenyl)benzamide and purified by preparative HPLC (H₂O,CH₃CN, 0.1% HCOOH). Method 3; Rt: 3.24 min. m/z: 424.12 (M+H)⁺. ¹H NMR (300 MHz, DMSO-d₆) δ ppm 6.42 (s, 2H) 7.14 (s, 1H) 7.47 (br s, 2H) 7.60 (dd, J=10.22, 6.56 Hz, 2H) 7.71 (s, 1H) 10.73 (br s, 1H).

### Example 7: 4-amino-N-(4-fluoro-3-(trifluoromethyl)phenyl)-3-sulfamoylbenzamide (E7)

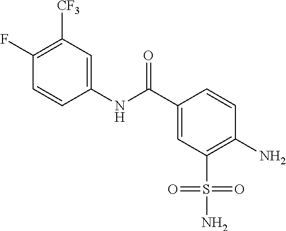 [SMILES: Nc1ccc(C(=O)Nc2ccc(F)c(C(F)(F)F)c2)cc1S(N)(=O)=O]

Similarly prepared according to the procedure described for the preparation of compound E3, starting from D10. Method 3; Rt=3.16 min, m/z=378.12 (M+H)⁺. ¹H NMR (300 MHz, DMSO-d6) δ ppm 6.47 (s, 2H) 6.88 (d, J=8.62 Hz, 1H) 7.36 (s, 2H) 7.50 (t, J=9.90 Hz, 1H) 7.91 (dd, J=8.67, 2.15 Hz, 1H) 8.05-8.13 (m, 1H) 8.22 (dd, J=6.69, 2.48 Hz, 1H) 8.29 (d, J=2.11 Hz, 1H) 10.34 (s, 1H).

### Example 8: 4-amino-N-(3-cyano-4-fluorophenyl)-3-sulfamoylbenzamide (E8)

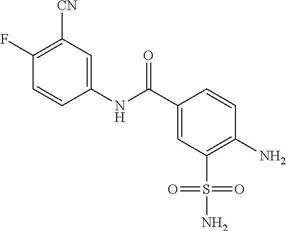 [SMILES: N#Cc1cc(NC(=O)c2ccc(N)c(S(N)(=O)=O)c2)ccc1F]

Similarly prepared according to procedure described for the preparation of E3, starting from D11. Method 3; Rt=2.65 min, m/z=334.95, (M+H)⁺. 1H NMR (300 MHz, DMSO-d₆) δ ppm 6.44 (br s, 2H) 6.88 (d, J=8.71 Hz, 1H) 7.37 (s, 2H) 7.53 (t, J=9.17 Hz, 1H) 7.90 (dd, J=8.62, 2.11 Hz, 1H) 7.99-8.13 (m, 1H) 8.25 (dd, J=5.78, 2.66 Hz, 1H) 8.28 (d, J=2.11 Hz, 1H) 10.36 (s, 1H).

### Example 9: 4-amino-N-(3-(difluoromethyl)-4-fluorophenyl)-3-sulfamoylbenzamide (E9)

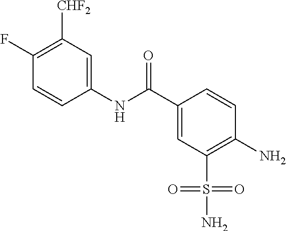 [SMILES: Nc1ccc(C(=O)Nc2ccc(F)c(C(F)F)c2)cc1S(N)(=O)=O]

Similarly prepared according to procedure described for the preparation of E3 starting from D12 LF_042_097. Method 3; Rt=2.85, m/z=360.08 (M+H)⁺. 1H NMR (300 MHz, DMSO-d6) δ ppm 6.45 (br s, 2H) 6.87 (d, J=8.62 Hz, 1H) 7.01-7.45 (m, 4H) 7.86-7.98 (m, 2H) 8.03-8.12 (m, 1H) 8.28 (d, J=2.11 Hz, 1H) 10.25 (s, 1H).

### Example 10: 4-amino-N-(3-chloro-4-fluorophenyl)-3-sulfamoylbenzamide (E10)

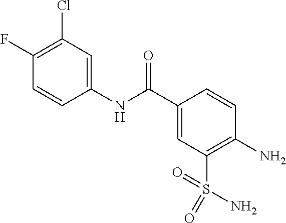 [SMILES: Nc1ccc(C(=O)Nc2ccc(F)c(Cl)c2)cc1S(N)(=O)=O]

A mixture of D23 (200. mg, 0,580 mmol), 1,4-dioxane (2 mL) and aqueous ammonia (2.3 mL, 10.38 mmol) were heated in a closed vial at 100° C. for 10 hrs. Solvent was removed, the residue suspended in DCM and filtered giving a off-white solid (110 mg). One half of this crude product was purified by preparative HPLC (H₂O/CH₃CN+1% TFA) giving the title compound E10 (8.4 mg) as off-white solid. Method 3; Rt: 2.98 min. m/z: 344.07 (M+H)⁺. ¹H NMR (300 MHz, DMSO-d₆) δ ppm 6.45 (br s, 2H) 6.87 (d, J=8.71 Hz, 1H) 7.31-7.43 (m, 3H) 7.64-7.77 (m, 1H) 7.89 (dd, J=8.67, 2.16 Hz, 1H) 8.05 (dd, J=6.92, 2.52 Hz, 1H) 8.26 (d, J=2.11 Hz, 1H) 10.20 (s, 1H).

### Example 11: 4-amino-N-(6-chloropyridin-3-yl)-3-sulfamoylbenzamide (E11)

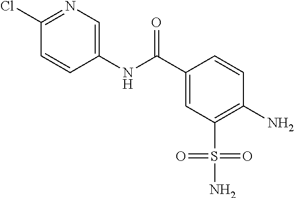 [SMILES: Nc1ccc(C(=O)Nc2ccc(Cl)nc2)cc1S(N)(=O)=O]

Similarly prepared according to procedure described for the preparation of E3 starting from D13. Method 3; Rt=2.33 min, m/z=327.05 (M+H)⁺. 1H NMR (300 MHz, DMSO-d6) δ ppm 6.49 (br s, 2H) 6.88 (d, J=8.71 Hz, 1H) 7.37 (s, 2H) 7.50 (d, J=8.71 Hz, 1H) 7.91 (dd, J=8.62, 2.02 Hz, 1H) 8.23 (dd, J=8.71, 2.75 Hz, 1H) 8.29 (d, J=2.02 Hz, 1H) 8.77 (d, J=2.57 Hz, 1H) 10.35 (s, 1H).

### Example 12: 4-amino-N-(4-fluoro-3-methylphenyl)-3-sulfamoylbenzamide (E12)

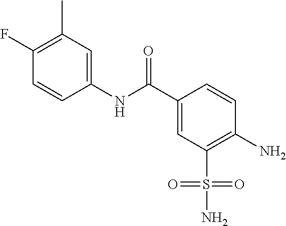 [SMILES: Cc1cc(NC(=O)c2ccc(N)c(S(N)(=O)=O)c2)ccc1F]

Similarly prepared according to procedure described for the preparation of E3, starting from D15. Method 3; Rt=2.83, m/z=324.14 (M+H)⁺. ¹H NMR (300 MHz, DMSO-d₆) δ ppm 2.23 (br d, J=1.60 Hz, 3H) 6.40 (s, 2H) 6.86 (d, J=8.62 Hz, 1H) 7.10 (t, J=9.26 Hz, 1H) 7.34 (s, 2H) 7.50-7.60 (m, 1H) 7.60-7.71 (m, 1H) 7.89 (dd, J=8.62, 2.11 Hz, 1H) 8.25 (d, J=2.11 Hz, 1H) 9.99 (s, 1H).

### Example 13: 4-amino-N-(3,5-difluoro-4-methylphenyl)-3-sulfamoylbenzamide (E13)

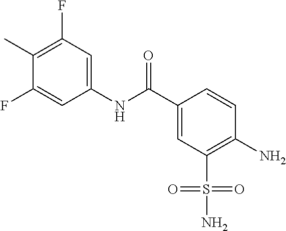 [SMILES: Cc1c(F)cc(NC(=O)c2ccc(N)c(S(N)(=O)=O)c2)cc1F]

Similarly prepared according to procedure described for the preparation of E3, starting from D16 LF_042_134. Method 3; Rt=3.17, m/z=342.25 (M+H)⁺. ¹H NMR (300 MHz, DMSO-d₆) δ ppm 2.11 (s, 3H) 6.47 (s, 2H) 6.87 (d, J=8.71 Hz, 1H) 7.36 (s, 2H) 7.44-7.57 (m, 2H) 7.88 (dd, J=8.67, 2.16 Hz, 1H) 8.25 (d, J=2.11 Hz, 1H) 10.25 (s, 1H).

### Example 14: 4-amino-3-sulfamoyl-N-(2,3,4-trifluorophenyl)benzamide (E14)

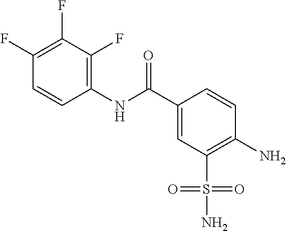 [SMILES: Nc1ccc(C(=O)Nc2ccc(F)c(F)c2F)cc1S(N)(=O)=O]

Similarly prepared according to procedure described for the preparation of E3, starting from D17. Method 3; Rt=2.65, m/z=346.17 (M+H)⁺. ¹H NMR (300 MHz, DMSO-d₆) δ ppm 6.47 (s, 2H) 6.87 (d, J=8.71 Hz, 1H) 7.22-7.47 (m, 4H) 7.88 (dd, J=8.71, 2.20 Hz, 1H) 8.27 (d, J=2.11 Hz, 1H) 10.11 (br s, 1H).

### Example 15: 4-amino-3-sulfamoyl-N-(2,4,5-trifluorophenyl)benzamide (E15)

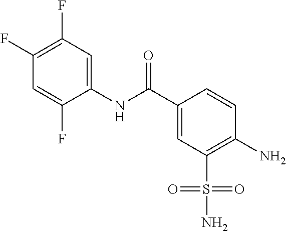 [SMILES: Nc1ccc(C(=O)Nc2cc(F)c(F)cc2F)cc1S(N)(=O)=O]

Similarly prepared according to procedure described for the preparation of E3, starting from D18. Method 3; Rt=2.68, m/z=346.17 (M+H)⁺. ¹H NMR (300 MHz, DMSO-d₆) δ ppm 6.47 (s, 2H) 6.87 (d, J=8.71 Hz, 1H) 7.22-7.47 (m, 4H) 7.88 (dd, J=8.71, 2.20 Hz, 1H) 8.27 (d, J=2.11 Hz, 1H) 10.11 (br s, 1H)

### Example 16: 4-amino-N-(2-chloro-4-fluorophenyl)-3-sulfamoylbenzamide (E16)

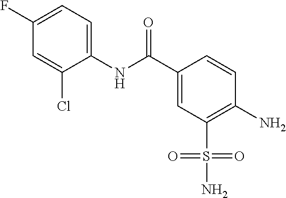 [SMILES: Nc1ccc(C(=O)Nc2ccc(F)cc2Cl)cc1S(N)(=O)=O]

Similarly prepared according to procedure described for the preparation of E3, starting from D19. ¹H NMR (300 MHz, DMSO-d₆) δ ppm 6.44 (br s, 2H) 6.87 (d, J=8.62 Hz, 1H) 7.22-7.31 (m, 1H) 7.35 (s, 2H) 7.47-7.62 (m, 2H) 7.89 (dd, J=8.80, 2.20 Hz, 1H) 8.27 (d, J=2.20 Hz, 1H) 9.85 (s, 1H)

### Example 17: 4-amino-2-methyl-5-sulfamoyl-N-(3,4,5-trifluorophenyl)benzamide (E17)

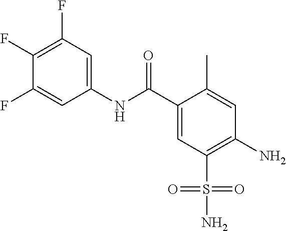 [SMILES: Cc1cc(N)c(S(N)(=O)=O)cc1C(=O)Nc1cc(F)c(F)c(F)c1]

D14 (150 mg, 0.390 mmol) was suspended in 1,4-Dioxane (1 mL) Aqueous ammonia (0.96 mL, 24.6 mmol) was added and the reaction mixture was stirred at 100° C. for 8 h and overnight at RT. More Aqueous ammonia (0.3 mL, 7.69 mmol) was added and the reaction mixture was stirred at 100° C. for another 8 h. The reaction was diluted with EtOAc and water, organic layer was dried over Na₂SO₄, filtered and concentrated under vacuo. The resulting crude were purified by preparative HPLC (H₂O/CH₃CN+1% TFA) to obtain, after lyophilization, the title compound E17 (110 mg) as white solid. Method 3; Rt=3.16 min, m/z=360.22 (M+H)⁺. ¹H NMR (300 MHz, DMSO-d₆) δ ppm 2.32 (s, 3H) 6.22 (br s, 2H) 6.68 (s, 1H) 7.26 (s, 2H) 7.56-7.70 (m, 2H) 7.74 (s, 1H) 10.49 (s, 1H).

### Example 18: (R)-4-amino-N-(3,4,5-trifluorophenyl)-3-(N-(1,1,1-trifluoropropan-2-yl)sulfamoyl)benzamide (E18)

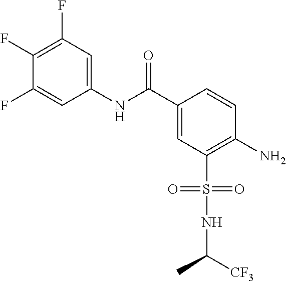 [SMILES: C[C@@H](NS(=O)(=O)c1cc(C(=O)Nc2cc(F)c(F)c(F)c2)ccc1N)C(F)(F)F]

Similarly prepared according to procedure described for the preparation of E3, starting from D25. Method 3; Rt: 3.75 min, m/z: 442.16 (M+H)⁺. ¹H NMR (300 MHz, DMSO-d₆) δ ppm 1.06 (d, J=6.88 Hz, 3H) 3.95-4.03 (m, 1H) 6.56 (br s, 2H) 6.92 (d, J=8.71 Hz, 1H) 7.58-7.80 (m, 2H) 7.91 (dd, J=8.71, 2.20 Hz, 1H) 8.25 (d, J=2.11 Hz, 1H) 8.55 (d, J=9.17 Hz, 1H) 10.35 (s, 1H).

### Example 19: (S)-4-amino-N-(3,4,5-trifluorophenyl)-3-(N-(1,1,1-trifluoropropan-2-yl)sulfamoyl)benzamide (E19)

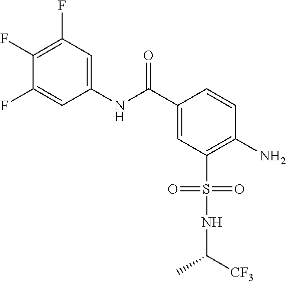 [SMILES: C[C@H](NS(=O)(=O)c1cc(C(=O)Nc2cc(F)c(F)c(F)c2)ccc1N)C(F)(F)F]

Similarly prepared according to procedure described for the preparation of E3, starting from D26. Method 3; Rt: 3.75 min, m/z: 442.16 (M+H)⁺. ¹H NMR (300 MHz, DMSO-d₆) δ ppm 1.06 (d, J=6.88 Hz, 3H) 3.95-4.00 (m, 1H) 6.57 (br s, 2H) 6.92 (d, J=8.80 Hz, 1H) 7.63-7.78 (m, 2H) 7.91 (dd, J=8.80, 2.20 Hz, 1H) 8.25 (d, J=2.20 Hz, 1H) 8.55 (d, J=9.17 Hz, 1H) 10.35 (s, 1H).

### Example 20: 4-amino-3-(N-cyclopropylsulfamoyl)-N-(3,4,5-trifluorophenyl)benzamide (E20)

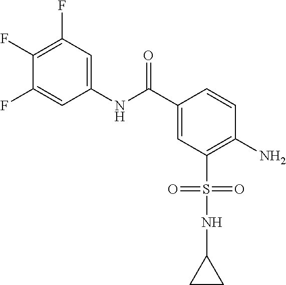 [SMILES: Nc1ccc(C(=O)Nc2cc(F)c(F)c(F)c2)cc1S(=O)(=O)NC1CC1]

A solution of D27 (125 mg, 0.32 mmol) in aqueous ammonia (0.38 mL, 3.22 mmol) and 1,4-dioxane (1.1 mL, 0.013 mol) was heated at 100° C. for 8 hrs in a closed vial. Solvent was removed in vacuo, the residue was treated with toluene and further evaporated by high vacuum pump, giving a residue (107 mg, off-white solid). A sample of this crude material (24.5 mg) was purified by preparative HPLC (H₂O/CH₃CN+1% TFA) to afford, after lyophilization the title compound E20 (13.2 mg) as white solid. Method 3; Rt: 3.55 min. m/z: 386.23 (M+H)⁺. ¹H NMR (300 MHz, DMSO-d₆) δ ppm 0.35-0.50 (m, 4H) 2.11 (td, J=6.51, 3.12 Hz, 1H) 6.54 (br s, 2H) 6.91 (d, J=8.71 Hz, 1H) 7.64-7.80 (m, 2H) 7.88 (dd, J=8.70, 2.00 Hz, 1H) 7.95 (dd, J=2.50 Hz, 1H) 8.25 (d, J=2.11 Hz, 1H) 10.35 (br s, 1H).

### Example 21: trans-4-amino-3-(N-(4-hydroxycyclohexyl)sulfamoyl)-N-(3,4,5-trifluorophenyl)benzamide (E21)

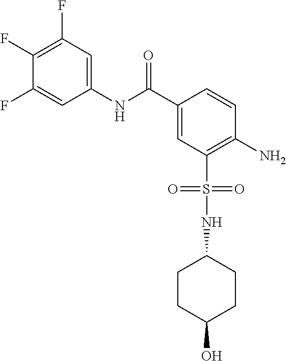 [SMILES: Nc1ccc(C(=O)Nc2cc(F)c(F)c(F)c2)cc1S(=O)(=O)N[C@H]1CC[C@H](O)CC1]

A solution of D28 (100 mg, 0.22 mmol) in aqueous ammonia (801.08 uL, 2.24 mmol) and 1,4-dioxane (1 mL) was heated at 100° C. for 8 hrs in a closed vial. Solvent was removed in vacuo, the residue was treated with toluene and further evaporated. Solvent traces were removed by high vacuum pump, giving a residue as off-white solid (108 mg). A sample of this crude material (20 mg) was purified by preparative HPLC (H₂O/CH₃CN+1% TFA), affording the title compound E21 (9 mg) as white solid. Method 3; Rt: 3.11. m/z: 444.25 (M+H)⁺. ¹H NMR (300 MHz, DMSO-d₆) δ ppm 0.87-1.35 (m, 4H) 1.50-1.86 (m, 4H) 2.78-2.97 (m, 1H) 3.20-3.34 (m, 2H) 6.51 (br s, 2H) 6.88 (d, J=8.71 Hz, 1H) 7.54-7.82 (m, 3H) 7.89 (dd, J=8.71, 2.11 Hz, 1H) 8.23 (d, J=2.11 Hz, 1H) 10.34 (s, 1H). ¹H NMR (300 MHz, DMSO-d₆+TFA) 6 ppm 0.87-1.37 (m, 4H) 1.45-1.86 (m, 4H) 2.79-3.01 (m, 1H) 3.14-3.43 (m, 1H) 6.88 (d, J=8.71 Hz, 1H) 7.55-7.82 (m, 3H) 0.00 (dd, J=8.50, 2.20 Hz, 1H) 8.23 (d, J=2.11 Hz, 1H) 10.33 (s, 1H).

### Example 22: cis-4-amino-3-(N-(4-hydroxycyclohexyl)sulfamoyl)-N-(3,4,5-trifluorophenyl)benzamide (E22)

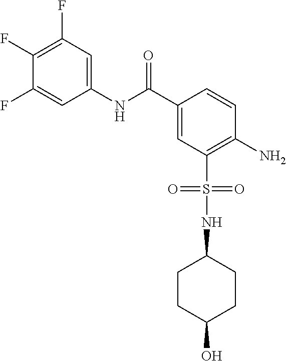 [SMILES: Nc1ccc(C(=O)Nc2cc(F)c(F)c(F)c2)cc1S(=O)(=O)N[C@H]1CC[C@@H](O)CC1]

Similarly prepared according to the procedure described for the preparation of compound E3, starting from D29. Method 3; Rt=3.24 min, m/z=444.25 (M+H)⁺. ¹H NMR (300 MHz, DMSO-d₆) δ ppm 1.35 (m, J=3.30 Hz, 4H) 1.43-1.62 (m, 4H) 2.82-3.08 (m, 1H) 3.49-3.58 (m, 2H) 6.52 (br s, 2H) 6.87 (d, J=8.62 Hz, 1H) 7.53-7.79 (m, 3H) 7.88 (dd, J=8.71, 2.29 Hz, 1H) 8.23 (d, J=2.20 Hz, 1H) 10.33 (s, 1H).

### Example 23: trans-4-amino-5-(N-(4-hydroxycyclohexyl)sulfamoyl)-2-methyl-N-(3,4,5-trifluorophenyl)benzamide (E23)

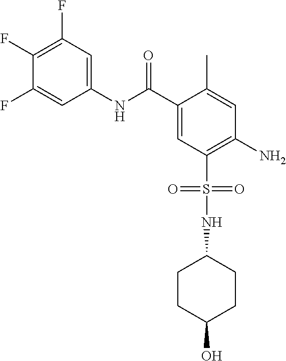 [SMILES: Cc1cc(N)c(S(=O)(=O)N[C@H]2CC[C@H](O)CC2)cc1C(=O)Nc1cc(F)c(F)c(F)c1]

Similarly prepared according to the procedure described for the preparation of compound E3, starting from D30 LF_042_146. Method 3; Rt=2.03 min, m/z=461.34 (M+H)⁺. ¹H NMR (300 MHz, DMSO-d₆) δ ppm 0.94-1.30 (m, 4H) 1.54-1.83 (m, 4H) 2.34 (s, 3H) 2.77-2.96 (m, 1H) 3.20-3.41 (m, 1H) 6.24 (br s, 2H) 6.68 (s, 1H) 7.54 (d, J=7.52 Hz, 1H) 7.58-7.70 (m, 2H) 7.73 (s, 1H) 10.46 (s, 1H).

### Example 24: cis-4-amino-3-(N-3-hydroxycyclobutyl)sulfamoyl)-N-(3,4,5-trifluorophenyl)benzamide (E24)

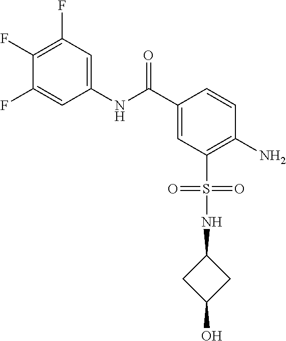 [SMILES: Nc1ccc(C(=O)Nc2cc(F)c(F)c(F)c2)cc1S(=O)(=O)N[C@H]1C[C@@H](O)C1]

Cis-4-fluoro-3-(N-(3-hydroxycyclobutyl)sulfamoyl)-N-(3,4,5-trifluorophenyl)benzamide was prepared according to the procedure described for the synthesis of D35, using cis-3-aminocyclobutanol instead of 3-aminocyclobutanol. The intermediate compound was further reacted with aqueous ammonia in 1,4-dioxane according to the procedure described for the preparation of E10. Method 3; Rt: 3.12. m/z: 416.29 (M+H)⁺. ¹H NMR (300 MHz, DMSO-d₆) δ ppm 1.43-1.74 (m, 2H) 2.11-2.26 (m, 2H) 2.94-3.17 (m, 1H) 3.59-3.71 (m, 1H) 6.55 (br s, 2H) 6.88 (d, J=8.71 Hz, 1H) 7.72 (dd, J=10.64, 6.60 Hz, 2H) 7.86-7.95 (m, 2H) 8.16-8.24 (m, 1H) 10.33 (s, 1H).

### Example 25: trans-4-amino-3-(N-3-hydroxycyclobutyl)sulfamoyl)-N-(3,4,5-trifluorophenyl)benzamide (E25)

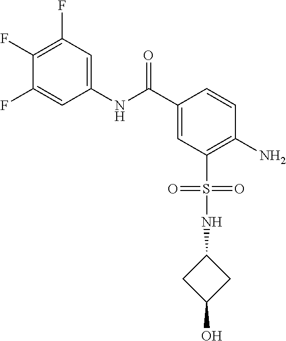 [SMILES: Nc1ccc(C(=O)Nc2cc(F)c(F)c(F)c2)cc1S(=O)(=O)N[C@H]1C[C@H](O)C1]

Trans-4-fluoro-3-(N-(3-hydroxycyclobutyl)sulfamoyl)-N-(3,4,5-trifluorophenyl)benzamide was prepared according to the procedure described for the synthesis of D35, using trans 3-aminocyclobutanol instead of 3-aminocyclobutanol. The intermediate compound was further reacted with aqueous ammonia in 1,4-dioxane according to the procedure described for the preparation of E10. Method 3; Rt: 3.04. m/z: 416.35 (M+H)⁺. ¹H NMR (300 MHz, DMSO-d₆) δ ppm 1.88 (br dd, J=7.93, 4.08 Hz, 2H) 1.92-2.04 (m, 2H) 3.70 (br d, J=7.70 Hz, 2H) 4.13 (br t, J=3.58 Hz, 1H) 6.54 (br s, 2H) 6.88 (d, J=8.71 Hz, 1H) 7.61-7.81 (m, 2H) 7.90 (dd, J=8.71, 2.20 Hz, 1H) 7.97 (d, J=8.16 Hz, 1H) 8.19 (d, J=2.20 Hz, 1H) 10.34 (s, 1H).

### Example 26: 4-amino-3-(N-((1R,3R)-3-hydroxycyclopentyl)sulfamoyl)-N-(3,4,5-trifluorophenyl)benzamide (E26)

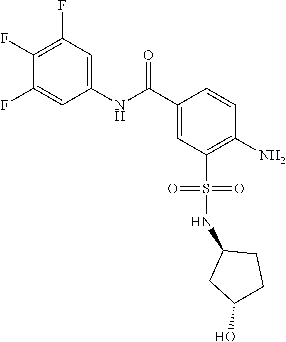 [SMILES: Nc1ccc(C(=O)Nc2cc(F)c(F)c(F)c2)cc1S(=O)(=O)N[C@H]1CC[C@H](O)C1]

A solution of D36 (105 mg, 0.24 mmol) in aqueous ammonia (0.5 mL, 4.6 mmol) and 1,4-Dioxane (0.5 mL) was heated for 8 hrs at 100° C. in a closed vial. The reaction solution was diluted with DCM/EtOAc (about 7/3) and water, then the organic layer was evaporated giving a residue (80 mg) that was purified by preparative HPLC (H₂O/CH₃CN+0.1% TFA). Method 3; Rt: 3.11. m/z: 430.27 (M+H)⁺. 1H NMR (300 MHz, DMSO-d₆) δ ppm 1.12-1.36 (m, 2H) 1.36-1.50 (m, 1H) 1.50-1.64 (m, 1H) 1.64-1.90 (m, 3H) 3.49-3.66 (m, 3H) 3.95-4.15 (m, 1H) 6.53 (br s, 2H) 6.89 (d, J=8.71 Hz, 1H) 7.66-7.77 (m, 3H) 7.90 (dd, J=8.71, 2.20 Hz, 1H) 8.23 (d, J=2.20 Hz, 1H) 10.35 (s, 1H)

### Example 27: tert-butyl 4-((2-amino-5-((3,4,5-trifluorophenyl)carbamoyl)phenyl)sulfonamido)piperidine-1-carboxylate (E27)

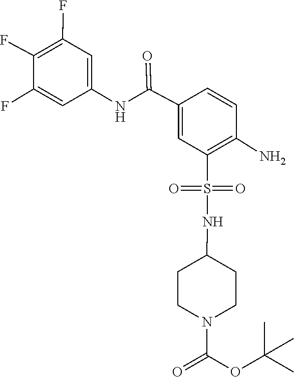 [SMILES: CC(C)(C)OC(=O)N1CCC(NS(=O)(=O)c2cc(C(=O)Nc3cc(F)c(F)c(F)c3)ccc2N)CC1]

A solution of D31 (147 mg, 0.28 mmol) in aqueous ammonia (0.99 mL, 2.77 mmol) and 1,4-dioxane (1.1 mL) was heated at 100° C. for 8 hrs in a closed vial. Solvent was removed in vacuo, the residue was treated with toluene and further evaporated. Traces of solvent were removed by high vacuum pump, giving a residue as off-white solid (107 mg). A sample of this crude material (20 mg) was purified by preparative HPLC (H₂O/CH₃CN+1% TFA) affording the title compound E27 (19.86 mg) as white solid. Method 3; Rt: 3.93 min. m/z: 529.09 (M+H)⁺. ¹H NMR (300 MHz, DMSO-d₆) δ ppm 1.13-1.32 (m, 2H) 1.36 (s, 9H) 1.47-1.65 (m, 2H) 2.75-2.95 (m, 2H) 3.05-3.23 (m, 1H) 3.67 (br d, J=13.57 Hz, 2H) 6.53 (br s, 2H) 6.89 (d, J=8.71 Hz, 1H) 7.64-7.78 (m, 2H) 7.82 (d, J=7.89 Hz, 1H) 7.90 (dd, J=8.67, 2.15 Hz, 1H) 8.24 (d, J=2.11 Hz, 1H) 10.34 (br s, 1H)

### Example 28: 4-amino-3-(N-(piperidin-4-yl)sulfamoyl)-N-(3,4,5-trifluorophenyl)benzamide (E28)

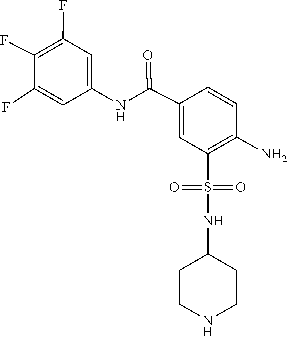 [SMILES: Nc1ccc(C(=O)Nc2cc(F)c(F)c(F)c2)cc1S(=O)(=O)NC1CCNCC1]

A solution of E27 in DCM (1 mL) was treated at room temperature with TFA (1 mL). The yellow reaction solution was magnetically stirred at room temperature for 1 h. Solvent was removed in vacuo and the residue purified by preparative HPLC (H₂O/CH₃CN+1‰TFA) affording the title compound E28 (8.6 mg) as TFA salt. ¹H NMR (300 MHz, DMSO-d₆) δ ppm 1.40-1.64 (m, 2H) 1.64-1.87 (m, 2H) 2.78-3.03 (m, 2H) 3.08-3.28 (m, 3H) 6.54 (br s, 2H) 6.91 (d, J=8.71 Hz, 1H) 7.72 (dd, J=10, 59, 6.56 Hz, 2H) 7.91 (dd, J=8.71, 2.11 Hz, 1H) 8.03 (d, J=7.61 Hz, 1H) 8.17 (br s, 1H) 8.25 (d, J=2.11 Hz, 1H) 8.41 (br s, 1H) 10.36 (br s, 1H). ¹H NMR (300 MHz, DMSO-d₆+TFA) 6 ppm 1.43-1.64 (m, 2H) 1.75 (br dd, J=13.75, 3.30 Hz, 2H) 2.79-3.00 (m, 2H) 3.08-3.22 (m, 2H) 3.22-3.38 (m, 1H) 6.91 (d, J=8.71 Hz, 1H) 7.61-7.81 (m, 2H) 7.91 (dd, J=8.71, 2.20 Hz, 1H) 8.03 (d, J=7.52 Hz, 1H) 8.13-8.34 (m, 2H) 8.35-8.60 (m, 1H) 10.35 (s, 1H). Method 3; Rt: 2.48 min. m/z: 429.26 (M+H)⁺.

### Example 29: 4-amino-3-((4-hydroxypiperidin-1-yl)sulfonyl)-N-(3,4,5-trifluorophenyl)benzamide (E29)

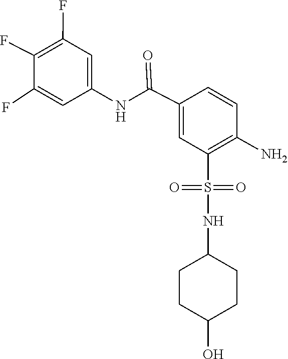 [SMILES: Nc1ccc(C(=O)Nc2cc(F)c(F)c(F)c2)cc1S(=O)(=O)NC1CCC(O)CC1]

A mixture of 4-fluoro-3-((4-hydroxypiperidin-1-yl)sulfonyl)-N-(3,4,5-trifluorophenyl)benzamide (prepared according to WO2013/096744) (50 mg, 0.11 mmol), 1,4-dioxane (150 uL) and aqueous ammonia (0.3 mL, 2.29 mmol) were heated at 100° C. for 8 hrs in a sealed tube. The reaction was diluted with EtOAc and water, organic layer was dried over Na₂SO₄, filtered and finally evaporated. The residue was suspended in DCM and filtered affording the title compound E29 (15.5 mg) as white solid. Method 3; Rt: 3.26 min. m/z: 430.2 (M+H)⁺. ¹H NMR (300 MHz, DMSO-d₆) δ ppm 1.30-1.52 (m, 2H) 1.60-1.83 (m, 2H) 2.77-2.92 (m, 2H) 3.25 (br s, 2H) 3.45-3.65 (m, 1H) 4.67 (d, J=3.76 Hz, 1H) 6.53-6.75 (m, 2H) 6.93 (d, J=8.71 Hz, 1H) 7.69 (dd, J=10, 50, 6.46 Hz, 2H) 7.81-7.98 (m, 1H) 8.08 (d, J=2.02 Hz, 1H) 10.31 (s, 1H)

### Example 30: 3-((4-hydroxypiperidin-1-yl)sulfonyl)-4-(methylamino)-N-(3,4,5-trifluorophenyl)benzamide (E30)

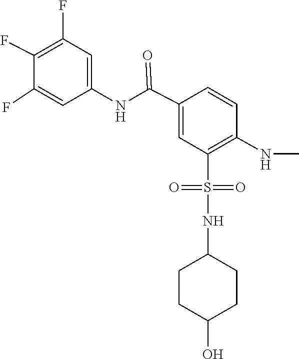 [SMILES: CNc1ccc(C(=O)Nc2cc(F)c(F)c(F)c2)cc1S(=O)(=O)NC1CCC(O)CC1]

A mixture of 3-((4-hydroxypiperidin-1-yl)sulfonyl)-N-(3,4,5-trifluorophenyl)benzamide (prepared according to WO2013/096744) (50 mg, 0.12 mmol), methanamine (1.04 mL, 2.08 mmol) in a DMSO (700 uL, 0.010 mol) and MeCN (140 uL, 0.003 mol) mixture was treated with triethylamine (480.87 uL, 3.47 mmol) and stirred at room temperature overnight. The reaction solution was purified by preparative HPLC (H₂O/CH₃CN+1% TFA) affording the title compound E30 (23 mg). ¹H NMR (300 MHz, DMSO-d₆) δ ppm 1.28-1.51 (m, 2H) 1.58-1.81 (m, 2H) 2.75-2.97 (m, 5H) 3.19-3.28 (m, 2H) 3.46-3.65 (m, 1H) 4.66 (d, J=3.90 Hz, 1H) 6.72-6.81 (m, 1H) 6.89 (d, J=8.90 Hz, 1H) 7.63-7.77 (m, 2H) 8.03-8.11 (m, 1H) 8.15 (d, J=2.11 Hz, 1H) 10.35 (s, 1H).

### Example 31: 4-amino-3-(N-(pyridin-4-yl)sulfamoyl)-N-(3,4,5-trifluorophenyl)benzamide (E31)

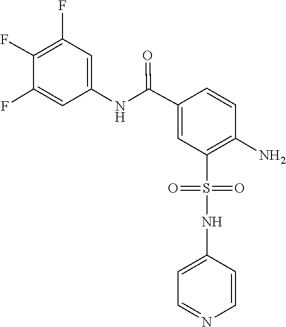 [SMILES: Nc1ccc(C(=O)Nc2cc(F)c(F)c(F)c2)cc1S(=O)(=O)Nc1ccncc1]

A 5 mL vial was charged with D32 (100 mg, 0.24 mmol) dioxane (1 mL) and aqueous ammonia (2 mL). The vial was sealed and heated for 8 hrs at 100° C. Solvents were removed by evaporation and the residue partitioned between water and EtOAc. The organic extract were combined and evaporated, the residue was dissolved in ½ dioxane/aqueous ammonia (2 mL) and heated in a closed vial for 8 hrs at 100° C. Solvent was removed in vacuo and the residue partitioned between water and EtOAc. The organic extract was evaporated and the residue was purified by flash chromatography on direct phase (EtOAc/MeOH). The fractions containing the product were combined and evaporated affording a residue (20 mg) that was purified by preparative HPLC (H₂O/CH₃CN+1% TFA) affording the title compound E31 as TFA salt (1.24 mg). Method 3; Rt: 2.63 min. m/z: 423.1 (M+H)⁺. ¹H NMR (300 MHz, DMSO-d₆) δ ppm 6.52 (br s, 1H) 6.82 (d, J=8.62 Hz, 1H) 7.03 (br d, J=6.88 Hz, 2H) 7.66-7.79 (m, 2H) 7.83 (dd, J=8.67, 2.16 Hz, 1H) 8.03 (d, J=6.00 Hz, 2H) 8.35 (d, J=2.11 Hz, 1H) 10.35 (s, 1H).

### Example 32: 4-amino-3-(N-(3-hydroxycyclobutyl)sulfamoyl)-N-(3,4,5-trifluorophenyl)benzamide (E32)

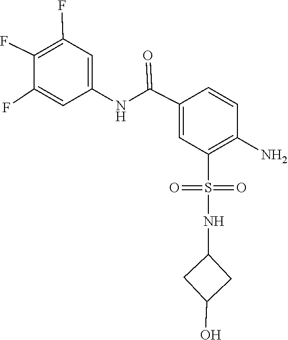 [SMILES: Nc1ccc(C(=O)Nc2cc(F)c(F)c(F)c2)cc1S(=O)(=O)NC1CC(O)C1]

Similarly prepared according to procedure described for the preparation of E10, starting from D35. Method 1; Rt: 1.81 min. m/z: 416.40 (M+H)⁺.

### Example 33: 4-amino-3-(N-(oxetan-3-yl)sulfamoyl)-N-(3,4,5-trifluorophenyl)benzamide (E33)

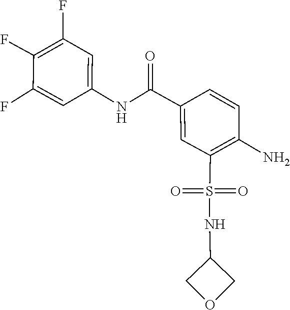 [SMILES: Nc1ccc(C(=O)Nc2cc(F)c(F)c(F)c2)cc1S(=O)(=O)NC1COC1]

Similarly prepared according to procedure described for the preparation of E10, starting from D33. Method 3; Rt: 3.20. m/z: 402.30 (M+H)⁺. 1H NMR (300 MHz, DMSO-d₆) δ ppm 4.23-4.43 (m, 3H) 4.44-4.54 (m, 2H) 6.58 (br s, 2H) 6.90 (d, J=8.71 Hz, 1H) 7.60-7.80 (m, 2H) 7.90 (dd, J=8.71, 2.20 Hz, 1H) 8.18 (d, J=2.20 Hz, 1H) 8.66 (br s, 1H) 10.36 (br s, 1H).

### Example 34: tert-butyl (S)-3-((2-amino-5-((3,4,5-trifluorophenyl)carbamoyl)phenyl)sulfonamido)pyrrolidine-1-carboxylate (E34)

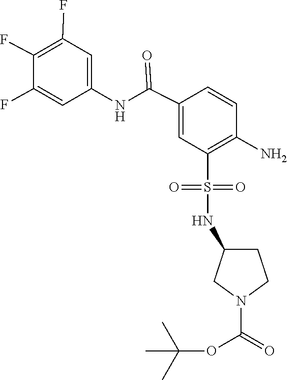 [SMILES: CC(C)(C)OC(=O)N1CC[C@H](NS(=O)(=O)c2cc(C(=O)Nc3cc(F)c(F)c(F)c3)ccc2N)C1]

Prepared similarly as described for the preparation of E10 using as starting material D34 and purified by preparative HPLC (H₂O/CH₃CN+0.1% HCOOH). Method 3; Rt: 3.81 min. m/z: 515.24 (M+H)⁺. ¹H NMR (300 MHz, DMSO-d₆) δ ppm 1.35 (br d, J=5.69 Hz, 9H) 1.55-1.77 (m, 1H) 1.78-2.00 (m, 1H) 2.88-3.05 (m, 1H) 3.07-3.31 (m, 4H) 3.64 (br s, 1H) 6.56 (br s, 2H) 6.91 (d, J=8.71 Hz, 1H) 7.64-7.79 (m, 2H) 7.92 (dd, J=8.71, 2.11 Hz, 1H) 8.07 (br s, 1H) 8.24 (d, J=2.02 Hz, 1H) 10.37 (s, 1H).

### Example E35: (S)-4-amino-3-(N-(pyrrolidin-3-yl)sulfamoyl)-N-(3,4,5-trifluorophenyl)benzamide (E35)

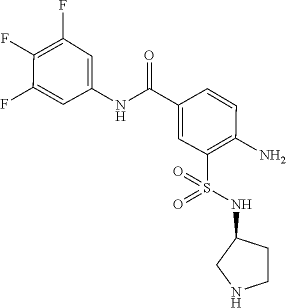 [SMILES: Nc1ccc(C(=O)Nc2cc(F)c(F)c(F)c2)cc1S(=O)(=O)N[C@H]1CCNC1]

A solution of E34 (30 mg, 0.06 mmol) in DCM (0.5 mL) was treated with trifluoroacetic acid (0.5 mL, 6.53 mmol) at room temperature. Solvent was removed in vacuo, giving a residue that was purified by preparative HPLC (H₂O/CH₃CN 0.1% TFA) giving the title compound E35 (10 mg) as white solid. Method 3; Rt: 2.47 min. m/z: 415.27 (M+H)⁺. ¹H NMR (300 MHz, DMSO-d₆) δ ppm 1.61-1.85 (m, 1H) 1.85-2.10 (m, 1H) 2.94 (br s, 1H) 3.17 (br s, 3H) 3.57-3.88 (m, 1H) 6.60 (br s, 2H) 6.95 (d, J=8.71 Hz, 1H) 7.63-7.80 (m, 2H) 7.95 (dd, J=8.76, 2.16 Hz, 1H) 8.16 (d, J=6.42 Hz, 1H) 8.24 (d, J=2.11 Hz, 1H) 8.47-8.68 (m, 1H) 8.70-9.02 (m, 1H) 10.38 (s, 1H).

### Example 36: 4-amino-3-methyl-5-sulfamoyl-N-(3,4,5-trifluorophenyl)benzamide (E36)

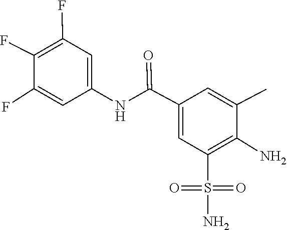 [SMILES: Cc1cc(C(=O)Nc2cc(F)c(F)c(F)c2)cc(S(N)(=O)=O)c1N]

A pressure vessel was charged with D38 (217 mg, 0.600 mmol), 1,4-dioxane (1.5 mL) and 33% aqueous ammonia (0.75 mL, 6.36 mmol). The pressure vessel was sealed and the reaction mixture was heated at 95° C. for 7.5 h, then stirred at RT overnight. More 33% aqueous ammonia (0.5 mL, 4.24 mmol) was added, and the reaction mixture was stirred at 100° C. for another 8.5 h. The reaction was diluted with EtOAc and water, organic layer was dried over Na₂SO₄, filtered and concentrated under vacuo. The residue was triturated with DCM, then was purified by preparative HPLC (H₂O/CH₃CN+1% TFA) to obtain, after lyophilisation the title compound E36 (86 mg) as off-white solid. Method 3; Rt=3.19 min, m/z=360.15 (M+H)⁺. ¹H NMR (300 MHz, DMSO-d₆) δ ppm 2.22 (s, 3H) 6.21 (s, 2H) 7.41 (s, 2H) 7.65-7.79 (m, 2H) 7.80-7.89 (m, 1H) 8.19 (d, J=2.02 Hz, 1H) 10.33 (s, 1H).

### Example 37: 6-amino-5-sulfamoyl-N-(3,4,5-trifluorophenyl)nicotinamide (E37)

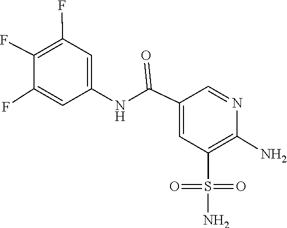 [SMILES: Nc1ncc(C(=O)Nc2cc(F)c(F)c(F)c2)cc1S(N)(=O)=O]

Similarly prepared according to the procedure described for the preparation of compound E3, starting from D40. Method 3: Rt=2.82 min, m/z=347.04 (M+H)⁺. ¹H NMR (300 MHz, DMSO-d₆) δ ppm 7.07 (br s, 2H) 7.60 (s, 2H) 7.65-7.81 (m, 2H) 8.46 (d, J=2.20 Hz, 1H) 8.80 (d, J=2.20 Hz, 1H) 10.48 (s, 1H).

The examples shown in Table 1 were prepared according to the synthetic methods described above. The general synthetic strategies, the appropriate intermediate materials and the relevant reaction steps (where appropriate), are indicated in Table 1 by referring to the appropriate Scheme.

TABLE 1

| | | | | Reaction Scheme, |
|---|---|---|---|---|
| | | | | Intermediate |
| | | | (ES+) | Material, Reaction |
| Example | Compound Name | Structure | m/z | Steps |
| E38 | 4-amino-3-(N-(3- (hydroxymethyl)oxetan-3- yl)sulfamoyl)-N-(3,4,5- trifluorophenyl)benzamide | 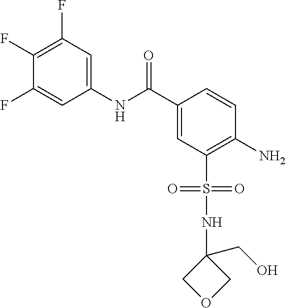 [SMILES: Nc1ccc(C(=O)Nc2cc(F)c(F)c(F)c2)cc1S(=O)(=O)NC1(CO)COC1] | 432 | Scheme 1 D7 → STEP F → STEP H |
| E39 | 4-amino-3-(N-((1- hydroxycyclohexyl)methyl) sulfamoyl)-N-(3,4,5- trifluorophenyl)benzamide | 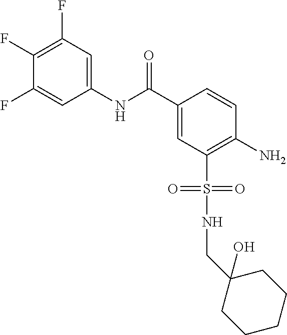 [SMILES: Nc1ccc(C(=O)Nc2cc(F)c(F)c(F)c2)cc1S(=O)(=O)NCC1(O)CCCCC1] | 458 | Scheme 1 D7 → STEP F → STEP H |
| E40 | 4-amino-N-(4-fluoro-3- methylphenyl)-2-methyl-5- sulfamoylbenzamide | 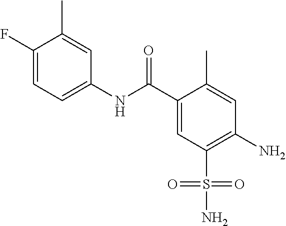 [SMILES: Cc1cc(NC(=O)c2cc(S(N)(=O)=O)c(N)cc2C)ccc1F] | 338 | Scheme 1 D6 → STEP B → STEP C |
| E41 | 4-amino-5-(N-((1r,4r)-4- hydroxycyclohexyl)sulfamoyl)- 2-methyl-N-(3,4,5- trifluorophenyl)benzamide | 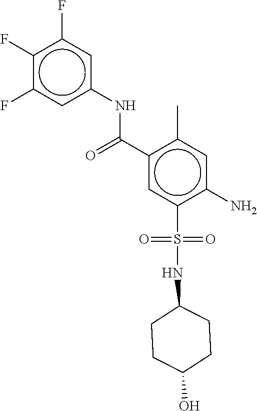 | 458 | Scheme 1 D14 → STEP F → STEP H |
| E42 | 4-amino-3-(N-(1-(pyridin- 2-yl)ethyl)sulfamoyl)-N- (3,4,5- trifluorophenyl)benzamide | 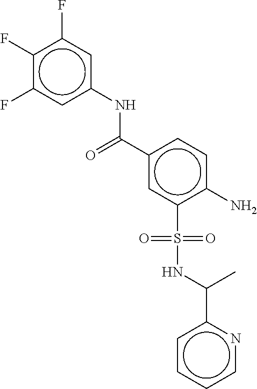 | 451 | Scheme 1 D7 → STEP F → STEP H |
| E43 | trans-4-amino-N-(3-chloro- 4-fluorophenyl)-3-(N-(4- hydroxycyclohexyl)sulfamoyl) benzamide | 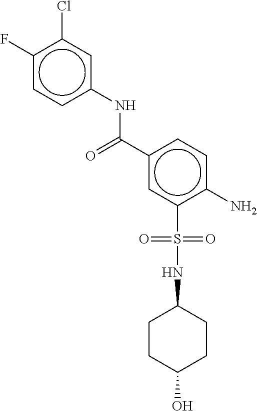 | 442 | Scheme 1 D9 → STEP F → STEP H |
| E44 | 4-amino-N-(3- (difluoromethyl)-4- fluorophenyl)-3-(N- ((1r,4r)-4- hydroxycyclohexyl)sulfamoyl) benzamide | 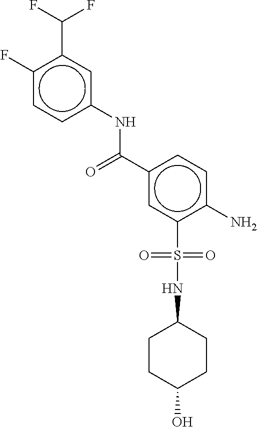 | 458 | Scheme 1 D12 → STEP F → STEP H |
| E46 | trans-4-amino-N-(3- (difluoromethyl)-4- fluorophenyl)-3-(N-(4- hydroxycyclohexyl)sulfamoyl) benzamide | 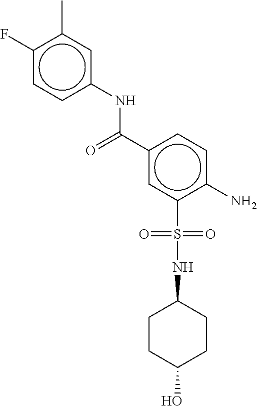 | 422 | D15 → STEP F → STEP H |
| E47 | 4-amino-3-(N-((1S,3R)-3- hydroxycyclopentyl)sulfamoyl)- N-(3,4,5- trifluorophenyl)benzamide | 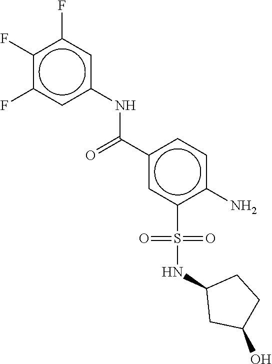 | 430 | Scheme 1 D7 → STEP F → STEP H |
| E48 | 4-amino-3-(N-(1,3- dihydroxypropan-2- yl)sulfamoyl)-N-(3,4,5- trifluorophenyl)benzamide | 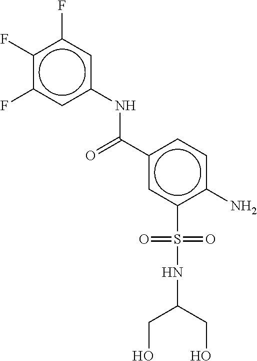 | 420 | Scheme 1 D7 → STEP F → STEP H |
| E49 | 4-amino-3-(N-((1R,3S)-3- hydroxycyclopentyl)sulfamoyl)- N-(3,4,5- trifluorophenyl)benzamide | 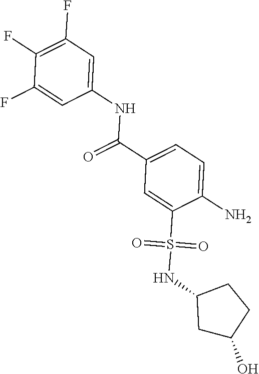 [SMILES: Nc1ccc(C(=O)Nc2cc(F)c(F)c(F)c2)cc1S(=O)(=O)N[C@@H]1CC[C@H](O)C1] | 430 | Scheme 14 D7 → STEP F → STEP H |
| E50 | 4-amino-3-((4-hydroxy-4- (hydroxymethyl)piperidin- 1-yl)sulfonyl)-N-(3,4,5- trifluorophenyl)benzamide | 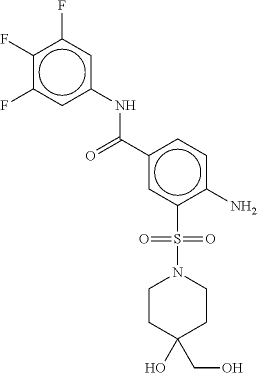 | 460 | Scheme 1 D7 → STEP F → STEP H |
| E51 | tert-butyl ((1R,2S)-2-((2- amino-5-((3,4,5- trifluorophenyl)carbamoyl) phenyl)sulfonamido)cyclo- pentyl)carbamate | 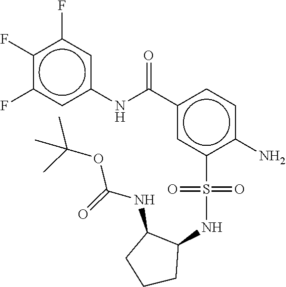 | 529 | Scheme 1 D7 → STEP F → STEP H |
| E52 | tert-butyl ((1S,2R)-2-((2- amino-5-((3,4,5- trifluorophenyl)carbamoyl) phenyl)sulfonamido)cyclo- pentyl)carbamate | 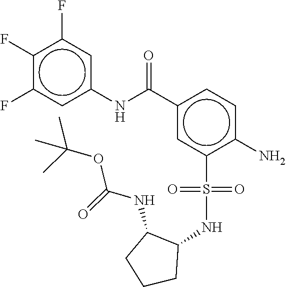 | 529 | Scheme 1 D7 → STEP F → STEP H |
| E53 | 4-amino-3-((3- hydroxypyrrolidin-1- yl)sulfonyl)-N-(3,4,5- trifluorophenyl)benzamide | 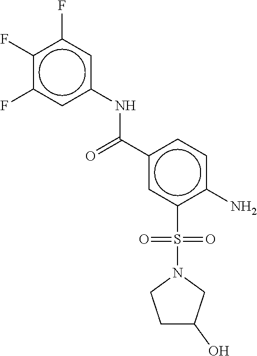 | 416 | Scheme 1 D7 → STEP F → STEP H |
| E54 | 4-amino-N-(3-chloro-4- fluorophenyl)-3-((4- hydroxypiperidin-1- yl)sulfonyl)benzamide | 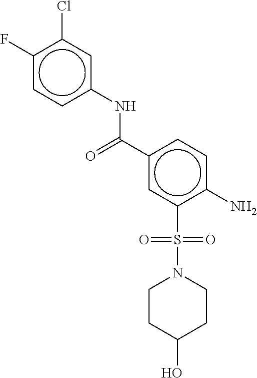 | 428 | Scheme 1 D9 → STEP F → STEP H |
| E55 | 4-amino-N-(3-chloro-4- fluorophenyl)-3-((3- hydroxyazetidin-1- yl)sulfonyl)benzamide | 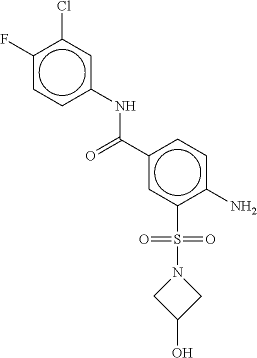 | 400 | Scheme 1 D9 → STEP F → STEP H |
| E56 | 4-amino-3-(N-(2,3- dihydroxypropyl)sulfamoyl)- N-(3,4,5- trifluorophenyl)benzamide | 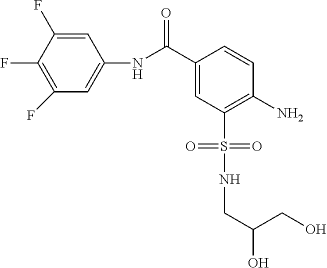 [SMILES: Nc1ccc(C(=O)Nc2cc(F)c(F)c(F)c2)cc1S(=O)(=O)NCC(O)CO] | 420 | Scheme 1 D7 → STEP F → STEP H |
| E57 | trans-4-amino-3-(N-(3- hydroxycyclopentyl)sulfamoyl)- N-(3,4,5- trifluorophenyl)benzamide | 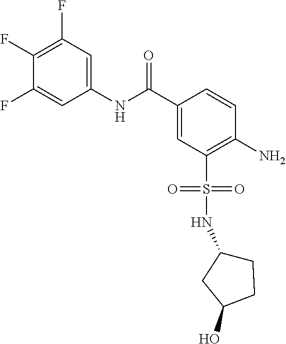 [SMILES: Nc1ccc(C(=O)Nc2cc(F)c(F)c(F)c2)cc1S(=O)(=O)N[C@@H]1CC[C@@H](O)C1] | 430 | Scheme 1 D7 → STEP F → STEP H |
| E58 | trans-6-amino-5-(N-(4- hydroxycyclohexyl)sulfamoyl)- N-(3,4,5- trifluorophenyl)nicotinamide | 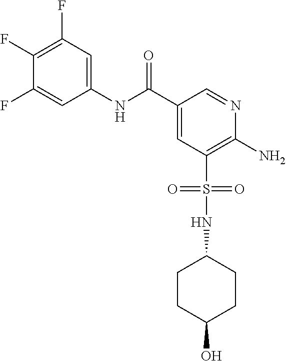 [SMILES: Nc1ncc(C(=O)Nc2cc(F)c(F)c(F)c2)cc1S(=O)(=O)N[C@H]1CC[C@H](O)CC1] | 445 | Scheme 3 |
| E59 | 2-amino-5-sulfamoyl-N- (3,4,5- trifluorophenyl)benzamide | 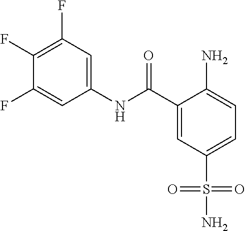 [SMILES: Nc1ccc(S(N)(=O)=O)cc1C(=O)Nc1cc(F)c(F)c(F)c1] | 346 | Scheme 4 |
| E60 | 4-amino-N-(3-chloro-4- fluorophenyl)-2-methyl-5- sulfamoylbenzamide | 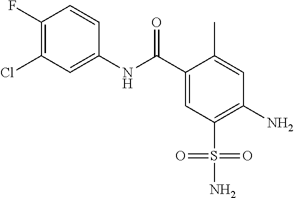 [SMILES: Cc1cc(N)c(S(N)(=O)=O)cc1C(=O)Nc1ccc(F)c(Cl)c1] | 358 | Scheme 1 D6 → STEP B → STEP C |
| E61 | trans-2-amino-5-(N-(4- hydroxycyclohexyl)sulfamoyl)- N-(3,4,5- trifluorophenyl)benzamide | 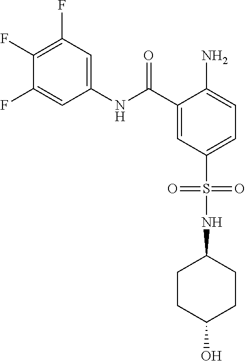 [SMILES: Nc1ccc(S(=O)(=O)N[C@H]2CC[C@H](O)CC2)cc1C(=O)Nc1cc(F)c(F)c(F)c1] | 444 | Scheme 4 |
| E62 | 2-amino-5-((4- hydroxypiperidin-1- yl)sulfonyl)- N-(3,4,5- trifluorophenyl)benzamide | 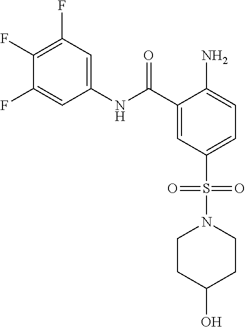 [SMILES: Nc1ccc(S(=O)(=O)N2CCC(O)CC2)cc1C(=O)Nc1cc(F)c(F)c(F)c1] | 430 | Scheme 4 |
| E63 | (R)-4-amino-2-methyl-N- (3,4,5-trifluorophenyl)-5- (N-(1,1,1-trifluoropropan- 2-yl)sulfamoyl)benzamide | 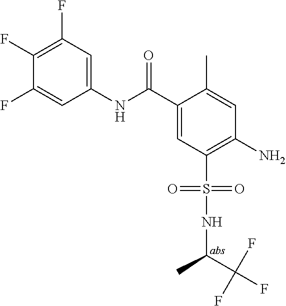 [SMILES: Cc1cc(N)c(S(=O)(=O)N[C@H](C)C(F)(F)F)cc1C(=O)Nc1cc(F)c(F)c(F)c1] | 456 | Scheme 1 D14 → STEP F → STEP H |
| E64 | (S)-4-amino-2-methyl-N- (3,4,5-trifluorophenyl)-5- (N-(1,1,1-trifluoropropan- 2-yl)sulfamoyl)benzamide | 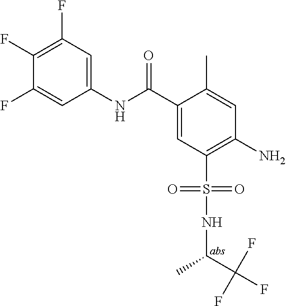 [SMILES: Cc1cc(N)c(S(=O)(=O)N[C@@H](C)C(F)(F)F)cc1C(=O)Nc1cc(F)c(F)c(F)c1] | 456 | Scheme 1 D14 → STEP F → STEP H |
| E65 | 4-amino-N-(3-chloro-4,5- difluorophenyl)-2-methyl- 5-sulfamoylbenzamide | 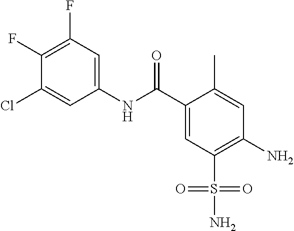 [SMILES: Cc1cc(N)c(S(N)(=O)=O)cc1C(=O)Nc1cc(F)c(F)c(Cl)c1] | 376 | Scheme 1 D6 → STEP B → STEP C |
| E66 | 4-amino-N-(6- chloropyridin-3-yl)-2- methyl-5- sulfamoylbenzamide | 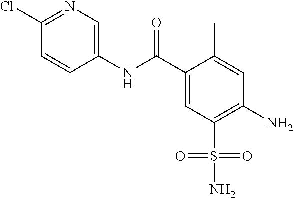 [SMILES: Cc1cc(N)c(S(N)(=O)=O)cc1C(=O)Nc1ccc(Cl)nc1] | 341 | Scheme 1 D6 → STEP B → STEP C |
| E67 | 4-amino-N-(4-fluoro-3- (trifluoromethyl)phenyl)-2- methyl-5- sulfamoylbenzamide | 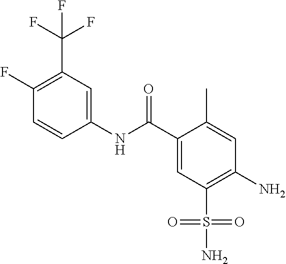 [SMILES: Cc1cc(N)c(S(N)(=O)=O)cc1C(=O)Nc1ccc(F)c(C(F)(F)F)c1] | 392 | Scheme 1 D6 → STEP B → STEP C |
| E68 | 4-amino-N-(3- (difluoromethyl)-4- fluorophenyl)-2-methyl-5- sulfamoylbenzamide | 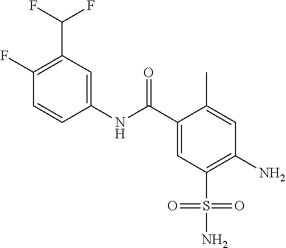 [SMILES: Cc1cc(N)c(S(N)(=O)=O)cc1C(=O)Nc1ccc(F)c(C(F)F)c1] | 374 | Scheme 1 D6 → STEP B → STEP C |
| E69 | 4-amino-N-(3-cyano-4- fluorophenyl)-2-methyl-5- sulfamoylbenzamide | 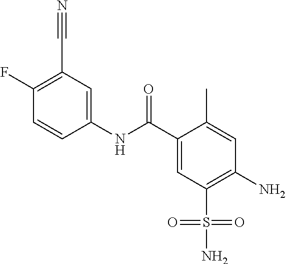 [SMILES: Cc1cc(N)c(S(N)(=O)=O)cc1C(=O)Nc1ccc(F)c(C#N)c1] | 349 | Scheme 1 D6 → STEP B → STEP C |

### Biology

### Assay

### Cells and Culture Conditions

HepAD38 cell line (Ladner et al., Antimicrob Agents Chemother, 1997, 41, 1715-20) was used for HBV inhibition assays. HepAD38 is a subclone, derived from hepatoblastoma cell line HepG2 (ATCC® Number: HB-8065™), that expresses HBV genome under the transcriptional control of a tetracycline-responsive promoter in a TET-OFF system: addition of tetracycline (TET) or doxycycline suppresses HBV replication, while its removal switches on the process allowing HBV viral particles release in the cell supernatant. HepAD38 cell line is maintained in DMEM/F12, supplemented with 10% of fetal bovine serum, 1% of glutamine, 1% of penicillin/streptomycin, 0.4 mg/ml G418 and 0.3 ug/ml tetracycline. For the HBV inhibition assay, doxycycline-free medium is used in order to allow virion production.

HepG2 hepatoma cells transfected with HBV plasmids corresponding to the different isolates were used to perform antiviral assay against different HBV genotypes. HBV plasmids were obtained by cloning fragment containing of 1.1-mer HBV genome into the SacI/SaII sites of pcDNA3.1zeo(−) plasmid (V86520, Thermofisher Scientific). The HepG2 cell line was maintained in DMEM, supplemented with 10% of FBS, 1% glutamine and 1% penicillin/streptomycin.

HepG2-NTCP hepatoma cell line was used to conduct the infection experiments. HepG2-NTCP was obtained by ectopic expression in HepG2 cells of the human Sodium Taurocholate Cotransporting Polypeptide receptor (hNTCP) using a lentiviral vector expressing human NTCP (Seeger et al., Molecular Therapy-Nucleic Acids, 2014). hNTCP was recently identified as HBV receptor (Yan H. et al., Elife. Nov. 13, 2012; 3). The complete viral life cycle of HBV could be obtained in the HepG2-NTCP cell line. HepG2-NTCP cells were maintained in DMEM, supplemented with 10% of FBS, 1% glutamine and 1% penicillin/streptomycin.

### Anti-HBV Activity In Vitro

HBV inhibition activity in vitro was performed in 96 multiwell plates. During the initial (primary) screening compounds were first tested in triplicates at concentrations of 0.1 μM, 0.5 μM and 1 μM. For selected compounds, an 8-point dose-response curve was obtained using 1:2 serial dilutions (starting from 2.5 μM, 1.25 μM or 0.4 μM, depending on the degree of inhibition observed during the primary screening). From the dose-response curves, half maximal effective concentration (EC₅₀) could be calculated (see also below).

In more detail, compounds—typically dissolved in DMSO stock solutions—were diluted to 2× the final desired concentration in 100 μl of the above medium (without doxycycline) and plated in three replicates in the 96-well plates.

Simultaneously, HepAD38 cells—extensively pre-washed in tetracycline-free medium in order to induce HBV production—were suspended at 2*10⁴ cells in 100 μl of tetracycline-free medium and added to each well of the plate, to yield a final assay volume of 200 μl.

DMSO, used for stock solutions and compounds dilutions, is always present in the assays at a final concentration of 0.5%.

Plates were then incubated 96 hours at 37° C. and then subjected to cell viability assays and extracellular HBV quantification, in order to evaluate both the cytotoxic potential and the anti-viral activity of compounds.

Cytotoxicity was assessed by a commercial fluorescence assay that measures the metabolic activity of cells, directly related to cell viability (Cell Titer Blue, Promega). For each compound, cytotoxicity was evaluated at the same concentration employed to evaluate its anti-HBV activity. Anti-HBV activity was evaluated by quantification of extracellular HBV DNA with direct qPCR. In particular, supernatant was collected and centrifuged for cell debris clarification, viral DNA was extracted from virions by addition of lysis buffer (1 mM 1,4-dithiothreitol, 0.2% sodium dodecyl sulphate) and incubated at 95° C. for 10 min. Samples were then diluted 1:40 and real time PCR amplification was performed with SYBR green assay (Power SYBR™ Green PCR Master Mix-Thermo Fisher Scientific) and specific HBV primer (HBV-DF:5′-ATTTGTTCAGTGGTTCGTAGGG-3′ (SEQ ID No. 1), HBV-DR:5′-CGGTAAAAAGGGACTCAAGATG-3′ (SEQ ID No. 2)).

HBV inhibition activity was determined on different HBV genotypes. For this purpose plasmids pcDNA3.1Zeo(−)-HBV1.1 expressing HBV genotypes A, B, C, D, E were transfected in HepG2 cell line and extracellular HBV quantification was performed by direct qPCR as described above. In detail, cells were seeded at 20,000 cells/well in polylisine coated 96-multiwell plates and 24 h later HBV plasmids were transfected with lipofection using manufacturer's procedure (Lipofectamine 3000 Reagent, Thermofisher Scientific). Five hours after transfection cells were extensively washed with PBS and compounds were added in 8-point dose-response curve in 0.5% DMSO as described above. After 4 days of incubation at 37° C. cells were subjected to cell viability assays and extracellular HBV quantification, in order to evaluate both the cytotoxic potential and the anti-viral activity of compounds, as described above. To avoid plasmid carry-over during real time PCR detection, the supernatant was treated with 1 Unit of DNAseI (DNase I Amplification Grade, Sigma) for 1 h at 37° C. before direct qPCR. Absence of plasmid contamination was verified by real-time PCR amplification with amp specific primers (AMP-F:5′-TGCTTAATCAGTGAGGCACCTA-3′ (SEQ ID No. 3), AMP-R:5′-AGCCCTCCCGTATCGTAGTTAT-3′ (SEQ ID No. 4)).

The ability of compounds to inhibit the establishment of HBV cccDNA in infected cells was investigated in an in vitro model of HBV infection. For this purpose HepG2-NTCP cell line, engineered to stably express the HBV receptor human sodium taurocholate cotrasporting polypeptide (hNTCP), was used and HBV particles were produced in HepAD38 following standard published procedure (Hepatitis B methods and protocols, Guo H., Cuconati, A.; 2017; Humana press). In detail HepG2-NTCP were seeded at 20.000 cells/well in collagen coated 96 multiwell plates. After 24 h, HBV particles produced as mentioned above were added to each well at 500 mge (multiplicity of genome equivalents, or number of genome for each cell) in 80 μl of complete medium containing 4% PEG and 2.5% DMSO. After 16 h the HBV particles were estensively washed with PBS and cells were incubated in 200 ul complete medium for 6 days at at 37° C. Compound treatment was performed in 12-point dose-response curve 1:2 diluted starting from 2 μM in 0.5% final DMSO as described above. Compounds were present during all the 6 days of the assay, starting from 3 h before viral particles addition. The ability of compounds to inhibit the establishment of HBV cccDNA was evaluated by quantification of extracellular HBeAg with ELISA (Elisa Kit HBE.CE. Manufacturer: DIA.PRO).

All HBV inhibition or antiviral activity data are typically reported in percent (%) relative to a non-treated reference sample. Excel and Graphpad Prism programs are typically used for data elaboration and EC₅₀ calculation.

### Pharmacokinetics

### Hepatocytes Stability Studies

Hepatic metabolism is often a major contributor to drug clearance from the body. Pooled cryopreserved human hepatocytes (commercially available for example from Bioreclamation IVT, LiverPool 20-donor Human Hepatocytes Product. No. X008000) are thawed and re-suspended in Hepatocyte Culture Medium (HCM) complete (commercially available, as for example from Lonza, HBM Hepatocytes Basal Medium Catalog No. CC-3199+HCM SingleQuots Catalog No. CC-4182). Compounds are diluted into the cell suspension (2.5 μl/2.5 mL, 1 million cells/mL) from 3 mM stock solution in DMSO to give a 3 μM concentration (0.1% DMSO). 150 μl (×2) of this mixture are taken and transferred into a 96-deep-well plate containing an equal volume of quenching solution (100% acetonitrile plus 0.1% formic acid) for time 0 incubation. Then 1 mL/well of the cell suspension-compound mixture (×2) are dispensed in 24-well plates. Incubations are performed at 37° C. in a DUBNOFF water bath, under low shaking. Compounds are tested at 6 time points in duplicate: 0, 30, 60, 120, 180 and 240 min. At each time point, an aliquot of 150 μl is taken, transferred into a 96-deep-well plate and then the reaction is stopped by addition of one volume of 100% acetonitrile plus 0.1% formic acid.

At 0, 120 and 240 min viability measurements by trypan blue exclusion test are performed. Samples are centrifuged at 1100×g for 30 min at +4° C. and 250 μl of the supernatant are transferred to a new 96-deep-well plate for bio analytical.

### Analytical Procedures:

The samples for analytical procedures are centrifuged at 4500 g at 4° C. for 5 minutes and split into two 96 deep-well plates.

The study samples are further n-fold diluted or dried under nitrogen at 25° C. and reconstituted according to the analytical method developed. Final plates are mixed for 10 min, sonicated for 5 min and the samples are injected into LC-MS/MS or LC-HRMS system. Sample analyses were performed using an API 4000QTrap Mass Spectrometer interfaced via the Turbo Ion Spray (ESI) to an LC system consisting of an Acquity UPLC Sample Manager autosampler and Acquity UPLC Binary Solvent Manager Pump or a Thermo Scientific Orbitrap QExactive interfaced to an LC system consisting of a Dionex Ultimated 3000 UHPLC.

### Results:

Stability is determined based on analysis of the disappearance of the compound as a function of incubation time, using area ratio (analyte peak area vs internal standard peak area). The elimination constant k is calculated by plotting mean disappearance values on a semi-logarithmic scale and fitting with a best fit linear regression. The half-life (t1/2) expressed in hours is derived using Equation 1: Equation 1: t1/2=ln 2/(−k). When the half-life could not be calculated, data are reported as: <0.5 or >4 hours. Intrinsic clearance given in μl/min/million cells is calculated using the Equation: Clint=KV/N. Where K=0.693/t1/2, V=incubation volume (ml) and N=number of hepatocytes/sample.

### Mouse PK Studies

C57BL/6 mice are used to evaluate plasma and liver exposure and pharmacokinetic parameters after intravenous and oral administration (from 2 to 200 mpk according to the compound tested and the administration route). 12 (+3 spare) healthy C57BL/6N male mice are obtained from Charles River S.p.A. Calco (Como), Italy. Animals are ordered weighing 21 to 27 grams and approximately 7 weeks old. Before and during testing, animals are housed in Individual Ventilated Cages (IVCs Tecniplast) with sawdust as bedding (three animals for cage). Cages are identified by a color code label recording the sample ID, animal number and details of treatment (route, dose and sex). Animals are identified by unique number on tail via permanent marker. Animal room controls are set to maintain temperature within the range from 20 to 24° C. and relative humidity within the range from 40 to 70% and an average daily airflow of at least 10 fresh air changes per hour. Actual conditions are recorded. The room is lit by fluorescent tubes controlled to give an artificial cycle of 12 hours light and 12 hours dark each day. All animals are weighed immediately before testing. Animals are dosed IV in a fed state and PO in a fasted state.

Compounds (from 0.4 to 20 mg/mL according to the compound tested and the administration route) are dissolved in DMSO/PEG400/H₂O (20/60/20) for IV administration and 0.5% Methocel E50 or 20% Hydroxypropyl-β-cyclodextrin (HP-β-CD) in citrate buffer pH=5 for PO administration. The appropriate dose volume of the test item, calculated for each animal according to body weight (administration volume: 5 mL/kg for IV or 5 mL/kg or 10 mL/kg for PO), is administered by injection into lateral tail vein using 2.5 mL syringe (BD Plasipak) for IV administration and administered orally by gavage using 5 mL Syringe (BD Plastipak) for PO administration. Whole blood sample (about 0.200 mL) is collected via retro orbital sinus using Isoflurane as anesthetic. Blood is collected in Li-Heparin Sartsted® gel tubes appropriately labeled indicating animal number and time point. Tubes are put in wet ice and then centrifuged within 15 minutes from blood collection. Centrifugation is performed using a Heraeus Multifuge® 3S/3S-R set, at 2200×g for 10 minutes, internal temperature is kept at 4° C. After the centrifugation, about 100 μL of plasma samples are obtained and immediately transferred to 1.5 mL Eppendorf tubes and frozen at −20° C. (24/24 h alarmed). Whole blood samples are collected at different times point (0-0.25-0.5-1-2-4-6-8-24 hours) after dosing and kept frozen (−20° C.) until assayed by LC/MS/MS. Livers are explanted at different time point (0-8-24 hours), washed with saline solution, transferred to Eppendorf tubes and frozen at −80° C. until assayed by LC/MS/MS. Analytical procedures: Plasma samples are extracted using Liquid Handling Robot Hamilton StarPlus by protein precipitation with acetonitrile. Then the samples are centrifuged (3000 rpm×15 min at 4° C.) and the supernatant transferred and dried under nitrogen. The samples are reconstituted in water/acetonitrile 90/10 or 50/50 and then injected directly into an UPLC column. Sample analyses are performed using an API 4000 or/and API 5000 or/and API 4000QTrap Mass Spectrometer interfaced via the Turbo Ion Spray (ESI) to an LC system consisting of an Acquity UPLC Sample Manager autosampler and Acquity UPLC Binary Solvent Manager Pump. The results are calculated using Analyst Software linear regression with 1/x*x weighting. The Assay Precision is calculated for the Quality Controls by Watson Lims database.

Cmax is the maximum compound concentration from oral dosing; Tmax is the time at which Cmax is reached; AUC (0-24) is the area under the concentration vs time curve from 0 to 24 hours; AUC extrap is the area under the curve (AUC) extrapolated to infinity, from dosing time based on the last observed concentration.

Pharmacokinetic Analysis: The plasma clearance (CLp) of the compounds is calculated (using Watson PK program) as the dose divided by the area under the plasma concentration-time curve from time zero to infinity (AUC_{0-∞}). The apparent half-life is estimated from the slope of the terminal phase of the log plasma concentration-time data. The volume of distribution (Vdss) is determined using the following noncompartmental method:

Vdss=(Dose IV×AUMC)/(AUC_{0-∞})2

where AUMC is the total area under the first moment of the drug concentration time curve from time zero to infinity. Bioavailability is estimated as the AUC_{0-∞} ratio following oral and intravenous administration, normalized for differences in dose.

### Results

The exemplified compounds described herein were tested in the assays described above. All the compounds displayed no measurable cytotoxicity at the tested compound concentration.

Results for HBV inhibition are reported in the following Table 2.

Legend: A indicates HBV inhibition greater than 50% at the concentration indicated in the table or EC₅₀ less than 1 μM; B indicates HBV inhibition less than 50% at the concentration indicated in the table or EC₅₀ greater than 1 μM.

TABLE 2
HBV inhibition results

| | | HBV | |
|---|---|---|---|
| | | inhibition % | HBV inh |
| Example | Compound Name | (conc μM) | EC₅₀ (μM) |
| E1 | 4-(methylamino)-3-sulfamoyl-N-(3,4,5- | B (1) | — |
| | trifluorophenyl)benzamide | | |
| E2 | 4-amino-3-(N-methylsulfamoyl)-N-(3,4,5- | A (1) | — |
| | trifluorophenyl)benzamide | | |
| E3 | 4-amino-3-sulfamoyl-N-(3,4,5- | A (0.5) | — |
| | trifluorophenyl)benzamide | | |
| E4 | 4-amino-N-(3,4-difluorophenyl)-3- | A (0.5) | — |
| | sulfamoylbenzamide | | |
| E5 | 4-amino-2-chloro-5-sulfamoyl-N-(3,4,5- | — | A |
| | trifluorophenyl)benzamide | | |
| E6 | 4-amino-2-bromo-5-sulfamoyl-N-(3,4,5- | A (1) | — |
| | trifluorophenyl)benzamide | | |
| E7 | 4-amino-N-(4-fluoro-3-(trifluoromethyl)phenyl)-3- | A (0.5) | — |
| | sulfamoylbenzamide | | |
| E8 | 4-amino-N-(3-cyano-4-fluorophenyl)-3- | A (0.5) | — |
| | sulfamoylbenzamide | | |
| E9 | 4-amino-N-(3-(difluoromethyl)-4-fluorophenyl)-3- | A (0.5) | — |
| | sulfamoylbenzamide | | |
| E10 | 4-amino-N-(3-chloro-4-fluorophenyl)-3- | A (0.5) | — |
| | sulfamoylbenzamide | | |
| E11 | 4-amino-N-(6-chloropyridin-3-yl)-3- | B (0.5) | — |
| | sulfamoylbenzamide | | |
| E12 | 4-amino-N-(4-fluoro-3-methylphenyl)-3- | A (0.5) | — |
| | sulfamoylbenzamide | | |
| E13 | 4-amino-N-(3,5-difluoro-4-methylphenyl)-3- | B (0.5) | — |
| | sulfamoylbenzamide | | |
| E14 | 4-amino-3-sulfamoyl-N-(2,3,4- | B (0.5) | — |
| | trifluorophenyl)benzamide | | |
| E15 | 4-amino-3-sulfamoyl-N-(2,4,5- | B (0.5) | — |
| | trifluorophenyl)benzamide | | |
| E16 | 4-amino-N-(2-chloro-4-fluorophenyl)-3- | B (0.5) | — |
| | sulfamoylbenzamide | | |
| E17 | 4-amino-2-methyl-5-sulfamoyl-N-(3,4,5- | — | A |
| | trifluorophenyl)benzamide | | |
| E18 | (R)-4-amino-N-(3,4,5-trifluorophenyl)-3-(N-(1,1,1- | A (0.5) | — |
| | trifluoropropan-2-yl)sulfamoyl)benzamide | | |
| E19 | (S)-4-amino-N-(3,4,5-trifluorophenyl)-3-(N-(1,1,1- | A (0.5) | — |
| | trifluoropropan-2-yl)sulfamoyl)benzamide | | |
| E20 | 4-amino-3-(N-cyclopropylsulfamoyl)-N-(3,4,5- | A (0.5) | — |
| | trifluorophenyl)benzamide | | |
| E21 | trans-4-amino-3-(N-(4-hydroxycyclohexyl)sulfamoyl)- | — | A |
| | N-(3,4,5-trifluorophenyl)benzamide | | |
| E22 | cis-4-amino-3-(N-(4-hydroxycyclohexyl)sulfamoyl)- | A (0.5) | — |
| | N-(3,4,5-trifluorophenyl)benzamide | | |
| E23 | trans-4-amino-5-(N-(4-hydroxycyclohexyl)sulfamoyl)- | A (0.5) | — |
| | 2-methyl-N-(3,4,5-trifluorophenyl)benzamide | | |
| E24 | cis-4-amino-3-(N-3-hydroxycyclobutyl)sulfamoyl)-N- | A (0.5) | — |
| | (3,4,5-trifluorophenyl)benzamide | | |
| E25 | trans-4-amino-3-(N-3-hydroxycyclobutyl)sulfamoyl)- | A (0.5) | — |
| | N-(3,4,5-trifluorophenyl)benzamide | | |
| E26 | 4-amino-3 -(N-((1R,3R)-3- | A (0.5) | — |
| | hydroxycyclopentyl)sulfamoyl)-N-(3,4,5- | | |
| | trifluorophenyl)benzamide | | |
| E27 | tert-butyl 4-((2-amino-5-((3,4,5- | B (0.5) | — |
| | trifluorophenyl)carbamoyl)phenyl)sulfonamido)piperidme- | | |
| | 1-carboxylate | | |
| E28 | 4-amino-3-(N-(piperidin-4-yl)sulfamoyl)-N-(3,4,5- | B (0.5) | — |
| | trifluorophenyl)benzamide | | |
| E29 | 4-amino-3-((4-hydroxypiperidin-1-yl)sulfonyl)-N- | A (0.5) | — |
| | (3,4,5-trifluorophenyl)benzamide | | |
| E30 | 3-((4-hydroxypiperidin-1-yl)sulfonyl)-4- | B (0.5) | — |
| | (methylamino)-N-(3,4,5-trifluorophenyl)benzamide | | |
| E31 | 4-amino-3-(N-(pyridin-4-yl)sulfamoyl)-N-(3,4,5- | B (0.5) | — |
| | trifluorophenyl)benzamide | | |
| E33 | 4-amino-3-(N-(oxetan-3-yl)sulfamoyl)-N-(3,4,5- | A (0.5) | — |
| | trifluorophenyl)benzamide | | |
| E34 | tert-butyl (S)-3-((2-amino-5-((3,4,5- | A (0.5) | — |
| | trifluorophenyl)carbamoyl)phenyl)sulfonamido)pyrrolidine- | | |
| | 1-carboxylate | | |
| E35 | (S)-4-amino-3-(N-(pyrrolidin-3-yl)sulfamoyl)-N- | B (0.5) | — |
| | (3,4,5-trifluorophenyl)benzamide | | |
| E36 | 4-amino-3-methyl-5-sulfamoyl-N-(3,4,5- | A (0.5) | — |
| | trifluorophenyl)benzamide | | |
| E37 | 6-amino-5-sulfamoyl-N-(3,4,5- | B (0.5) | — |
| | trifluorophenyl)nicotinamide | | |
| E38 | 4-amino-3-(N-(3-(hydroxymethyl)oxetan-3- | A (0.5) | — |
| | yl)sulfamoyl)-N-(3,4,5-trifluorophenyl)benzamide | | |
| E39 | 4-amino-3-(N-((1- | A (0.5) | — |
| | hydroxycyclohexyl)methyl)sulfamoyl)-N-(3,4,5- | | |
| | trifluorophenyl)benzamide | | |
| E40 | 4-amino-N-(4-fluoro-3-methylphenyl)-2-methyl-5- | A | |
| | sulfamoylbenzamide | | |
| E41 | 4-amino-5-(N-((1R,4R)-4- | A (0.5) | — |
| | hydroxycyclohexyl)sulfamoyl)-2-methyl-N-(3,4,5- | | |
| | trifluorophenyl)benzamide | | |
| E42 | 4-amino-3-(N-(1-(pyridin-2-yl)ethyl)sulfamoyl)-N- | B (0.5) | — |
| | (3,4,5-trifluorophenyl)benzamide | | |
| E43 | trans-4-amino-N-(3-chloro-4-fluorophenyl)-3-(N-(4- | A (0.5) | — |
| | hydroxycyclohexyl)sulfamoyl)benzamide | | |
| E44 | 4-amino-N-(3-(difluoromethyl)-4-fluorophenyl)-3-(N- | A (0.5) | — |
| | ((1R,4R)-4-hydroxycyclohexyl)sulfamoyl)benzamide | | |
| E46 | trans-4-amino-N-(3-(difluoromethyl)-4-fluorophenyl)- | A (0.5) | — |
| | 3-(N-(4-hydroxycyclohexyl)sulfamoyl)benzamide | | |
| E47 | 4-amino-3-(N-((1S,3R)-3- | A (0.5) | — |
| | hydroxycyclopentyl)sulfamoyl)-N-(3,4,5- | | |
| | trifluorophenyl)benzamide | | |
| E48 | 4-amino-3-(N-(1,3-dihydroxypropan-2-yl)sulfamoyl)- | B (0.5) | — |
| | N-(3,4,5-trifluorophenyl)benzamide | | |
| E49 | 4-amino-3-(N-((1R,3S)-3- | A (0.5) | — |
| | hydroxycyclopentyl)sulfamoyl)-N-(3,4,5- | | |
| | trifluorophenyl)benzamide | | |
| E50 | 4-amino-3-((4-hydroxy-4-(hydroxymethyl)piperidin-1- | A (0.5) | — |
| | yl)sulfonyl)-N-(3,4,5-trifluorophenyl)benzamide | | |
| E51 | tert-butyl ((1R,2S)-24(2-amino-5-((3,4,5- | A (1) | — |
| | trifluorophenyl)carbamoyl)phenyl)sulfonamido) | | |
| | cyclopentyl)carbamate | | |
| E52 | tert-butyl ((1S,2R)-24(2-amino-5-((3,4,5- | A (1) | — |
| | trifluorophenyl)carbamoyl)phenyl)sulfonamido) | | |
| | cyclopentyl)carbamate | | |
| E53 | 4-amino-3-((3-hydroxypyrrolidin-1-yl)sulfonyl)-N- | A (0.5) | — |
| | (3,4,5-trifluorophenyl)benzamide | | |
| E54 | 4-amino-N-(3-chloro-4-fluorophenyl)-3-((4- | A (0.5) | — |
| | hydroxypiperidin-1-yl)sulfonyl)benzamide | | |
| E55 | 4-amino-N-(3-chloro-4-fluorophenyl)-3-((3- | A (0.5) | — |
| | hydroxyazetidin-1-yl)sulfonyl)benzamide | | |
| E56 | 4-amino-3-(N-(2,3-dihydroxypropyl)sulfamoyl)-N- | A (0.5) | — |
| | (3,4,5-trifluorophenyl)benzamide | | |
| E57 | trans-4-amino-3-(N-(3- | A (0.5) | — |
| | hydroxycyclopentyl)sulfamoyl)-N-(3,4,5- | | |
| | trifluorophenyl)benzamide | | |
| E58 | trans-6-amino-5-(N-(4-hydroxycyclohexyl)sulfamoyl)- | B (0.1) | — |
| | N-(3,4,5-trifluorophenyl)nicotinamide | | |
| E59 | 2-amino-5-sulfamoyl-N-(3,4,5- | B (0.5) | — |
| | trifluorophenyl)benzamide | | |
| E60 | 4-amino-N-(3-chloro-4-fluorophenyl)-2-methyl-5- | — | A |
| | sulfamoylbenzamide | | |
| E61 | trans-2-amino-5-(N-(4-hydroxycyclohexyl)sulfamoyl)- | — | A |
| | N-(3,4,5-trifluorophenyl)benzamide | | |
| E62 | 2-amino-5-((4-hydroxypiperidin-1-yl)sulfonyl)- | — | A |
| | N-(3,4,5-trifluorophenyl)benzamide | | |
| E63 | (R)-4-amino-2-methyl-N-(3,4,5-trifluorophenyl)-5-(N- | A (0.1) | — |
| | (1,1,1-trifluoropropan-2-yl)sulfamoyl)benzamide | | |
| E64 | (S)-4-amino-2-methyl-N-(3,4,5-trifluorophenyl)-5-(N- | A (0.5) | — |
| | (1,1,1-trifluoropropan-2-yl)sulfamoyl)benzamide | | |
| E65 | 4-amino-N-(3-chloro-4,5-difluorophenyl)-2-methyl-5- | A (0.1) | — |
| | sulfamoylbenzamide | | |
| E66 | 4-amino-N-(6-chloropyridin-3-yl)-2-methyl-5- | B (0.5) | — |
| | sulfamoylbenzamide | | |
| E67 | 4-amino-N-(4-fluoro-3-(trifluoromethyl)phenyl)-2- | A (0.5) | — |
| | methyl-5-sulfamoylbenzamide | | |
| E68 | 4-amino-N-(3-(difluoromethyl)-4-fluorophenyl)-2- | A (0.1) | — |
| | methyl-5-sulfamoylbenzamide | | |
| E69 | 4-amino-N-(3-cyano-4-fluorophenyl)-2-methyl-5- | A (0.5) | — |
| | sulfamoylbenzamide | | |

Results in Table 2 clearly indicate that the compound of the invention display anti-HBV activity. Antiviral activity of compound E17 was evaluated against different HBV genotypes and in an in vitro infection model. Results are represented respectively as HBV inhibition EC₅₀ and HBeAg inhibition EC₅₀ in Tables 3 and 4. Data are indicated as mean and standard deviation of at least three independent experiments.

TABLE 3
Antiviral activity of E17 against different HBV genotypes

| | HBV inhibition EC₅₀ |
|---|---|
| Genotype | (Mean ± StD, μM) |
| A | 0.0582 ± 0.0254 |
| B | 0.0498 ± 0.0179 |
| C | 0.0282 ± 0.0108 |
| D | 0.0387 ± 0.0111 |
| E | 0.0512 ± 0.0136 |

In Table 3 above, the EC₅₀ of HBV inhibition of compound E17 is reported for each HBV genotype. Data are indicated as mean and standard deviation of at least three independent experiments.

TABLE 4
HBeAg inhibition EC₅₀ by E17

| | | HBeAg* inhibition EC₅₀ |
|---|---|---|
| | | (Mean ± StD, μM) |
| | | *surrogate of |
| Example | Compound Name | cccDNA biogenesis |
| E17 | 4-amino-2-methyl-5-sulfamoyl-N- | 0.147 ± 0.023 |
| | (3,4,5-trifluorophenyl)benzamide | |

In Table 4 above, the EC₅₀ of HBeAg inhibition of compound E17 is reported. Data is indicated as mean and standard deviation of at least three independent experiments. It is interesting to note that the above compound displays conserved activity among different genotypes maintaining the same potency. Interestingly, using the release of HBeAg as a surrogate of the establishment of cccDNA, as reported in Zhou T. et al Antiviral Res. 2006, the compound displays inhibition activity on the cccDNA biogenesis with EC₅₀ of 0.147 μM, thus showing an important effect for the complete eradication of HBV virus.

### In Vivo Properties

Compounds of the invention were evaluated in in vitro and in vivo pharmacokinetic studies. Compound 4-amino-3-sulfamoyl-N-(3,4,5-trifluorophenyl)benzamide (E3) is stable in mouse and human hepatocytes (data not shown). When dosed in vivo in mice, the compound showed low in vivo clearance (10 mL/min/Kg).

Plasma PK parameters and plasma and liver concentrations after PO administration in mice at 100 mpk in 0.5% methocell were evaluated for compound E3 and are summarized in Table 5, Table 6 and Table 7 below.

TABLE 5
Plasma PK parameters for Compound E3

| | Parameter | | | | | |
|---|---|---|---|---|---|---|
| | | Original | | | AUC | AUC |
| | | Dose | Cmax | Tmax | (0-24 h) | Extrap |
| | | Units | | | | |
| | Route | mg/kg | μM | Hours | μM*Hours | μM*Hours |
| Subject 01 | PO | 100 | 13.0 | 2.00 | 160 | 161.7 |
| Subject 02 | PO | 100 | 17.2 | 1.00 | 171 | 172.2 |
| Subject 03 | PO | 100 | 17.0 | 2.00 | 186 | 186.4 |
| Mean | | | 16 | 1.7 | 172 | 173 |

TABLE 6
Plasma concentrations (uM) of compound E3 after
PO administration at 100 mpk in 0.5% methocell

| | Hours | | | | | | | |
|---|---|---|---|---|---|---|---|---|
| Subject | 0.25 | 0.5 | 1 | 2 | 4 | 6 | 8 | 24 |
| 01 | 6.15 | 7.77 | 10.2 | 13.0 | 10.9 | 11.9 | 9.01 | 0.268 |
| 02 | 5.05 | 7.23 | 17.2 | 10.7 | 11.2 | 10.5 | 10.4 | 0.184 |
| 03 | 3.95 | 10.6 | 9.61 | 17.0 | 10.6 | 9.06 | 12.1 | 0.0502 |
| Mean | 5 | 9 | 12 | 14 | 11 | 10 | 10 | 0.2 |

!TABLE 7
Liver concentrations (μM) of
compound E3 after PO administration
at 100 mpk in 0.5% methocell

| | Hours | |
|---|---|---|
| Subject | 8 | 24 |
| 01 | 125.87 | 3.0342 |
| 02 | 119.96 | 1.8500 |
| 03 | 142.60 | 0.62717 |
| Mean | 129 | 2 |

As reported in the tables 6 and 7, E3 liver levels (8 h) after PO administration are 13 fold higher than plasma. Data represent the ratio between the liver and plasma concentration at the same time point (8 h).

Plasma PK parameters and liver concentrations after PO administration in mice at different doses were also evaluated for compound E17. Results are reported in Table 8 below.

TABLE 8
Liver concentrations and liver/plasma concentration
of compound E17 after PO administration
at the indicated dose in 0.5% methocell

| Dose, p.o. (mg/kg) | Liver [mM] | [Liver]/[Plasma] |
|---|---|---|
| 25 | 317 | 15.9 |
| 75 | 747 | 14.4 |
| 150 | 677 | 13.8 |

The high liver-to-plasma concentration of E3 and E17 is an important factor to be considered given that the liver is the principal tissue affected by hepatitis B disease. HBV inhibitors with hepatoselective distribution profiles represent an important strategy in developing safe drug candidates (Tu M. et al., Current Topics in Medicinal Chemistry, 2013, 13, 857-866).

1. A compound of general formula (I): 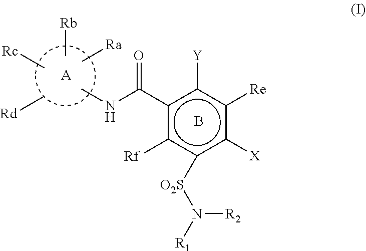
wherein:
A is a 6-membered aromatic or heteroaromatic ring;
B is a 6-membered aryl optionally containing one or more N atoms;
X is H or NR₃R₄;
Y is selected from the group consisting of hydrogen, halogen, C₁₋₆alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;
with the proviso that, when X is H, Y is selected form the group consisting of NH₂, NH(C₁₋₆alkyl), N(CH₃)₂, NHC(O)CH₃;
R₁ and R₂ are each independently selected from H, linear or branched C₁₋₆alkyl, saturated or partially unsaturated
C₃₋₇ cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀ heterocycloalkyl;
or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl and (CH₂)ₙR₅;
each occurrence of n is independently 0, 1, 2, 3 or 4;
R₃ and R₄ are each independently H, or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;
R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;
Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O) CH₃, OH and CN; or is absent;
Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rd is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Re is selected from the group consisting of hydrogen, halogen, C₁₋₃alkyl; or is absent;
Rf is hydrogen, halogen, C₁₋₃alkyl; or is absent;
provided that the compound is not 2-amino-N-(4-chloro-2-methylphenyl)-5-sulfamoylbenzamide or N-(2-methoxyphenyl)-2-(methylamino)-5-(piperidin-1-ylsulfonyl)benzamide;
and pharmaceutically acceptable salts, tautomers, isomers, stereoisomers thereof.

2. The compound according to claim 1 having formula (Ia): 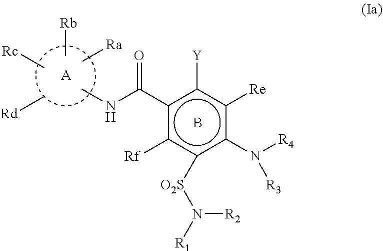
wherein:
A is a 6-membered aromatic or heteroaromatic ring;
B is a 6-membered aryl optionally containing one or more N atoms;
Y is selected from the group consisting of hydrogen, halogen, C₁₋₃alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;
R₁ and R₂ are each independently selected from H, linear or branched C₁₋₆alkyl, saturated or partially unsaturated
C₃₋₇cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀ heterocycloalkyl;
or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl, (CH₂)ₙR₅;
each occurrence of n is independently 0, 1, 2, 3 or 4;
R₃ and R₄ are each independently H or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;
R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;
Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rd is selected from the group consisting hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Re is selected from the group consisting of hydrogen, halogen and C₁₋₃alkyl; or is absent;
Rf is hydrogen, halogen and C₁₋₃alkyl; or is absent;
and pharmaceutically acceptable salts, tautomers, isomers, stereoisomers thereof.

3. The compound according to claim 1, wherein:
A is a 6-membered aromatic or heteroaromatic ring;
B is a 6-membered aryl optionally containing one or more N atoms;
Y is selected from the group consisting of hydrogen, halogen, C₁₋₃alkyl, NH₂, NH(C₁₋₆alkyl), N(CH₃)₂, NHC(O)CH₃, OH and CN or is absent;
R₁ is H, linear or branched C₁₋₆alkyl, cyclopropyl, cyclobutyl, cyclopentyl, cyclohexyl, piperidinyl, pirrolidinyl, oxetanyl, tetrahydrofuranyl, pyridinyl, said C₁₋₆alkyl, cyclopropyl, cyclobutyl, cyclopentyl, cyclohexyl, piperidinyl, pirrolidinyl, oxetanyl, tetrahydrofuranyl or pyridinyl being optionally substituted with one or more substituents selected from OH, halogen, NH₂, NH(C═O)OC₁₋₆alkyl, NH(C₁₋₆alkyl), C₁₋₆hydroxyalkyl, 5- or 6-membered heteroaryl, C(═O)C₁₋₆alkyl, C(═O)OC₁₋₆alkyl, OC₁-6alkyl;
R₂ is H or methyl;
or R₁ and R₂ taken together form with the N atom to which they are attached a heterocyclic ring selected from piperidine, pirrolidine, morpholine, thiomorpholine and piperazine, said ring being optionally substituted with one or more substituents selected from halogen, C₁₋₃alkyl, OH and CH₂R₅;
R₃ and R₄ are each independently H or C₁₋₃alkyl; in particular hydrogen or methyl;
R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;
Ra is selected from the group consisting of hydrogen, halogen, C₁₋₃alkyl, haloC₁₋₃alkyl and CN; or is absent;
Rb is selected from the group consisting of hydrogen, halogen, C₁₋₃alkyl, haloC₁₋₃alkyl and CN; or is absent;
Rc is selected from the group consisting of hydrogen, halogen, C₁₋₃alkyl, haloC₁₋₃alkyl and CN; or is absent;
Rd is selected from the group consisting of hydrogen, halogen, C₁₋₃alkyl, haloC₁₋₃alkyl and CN; or is absent;
Re is selected from the group consisting of hydrogen, halogen, C₁₋₃alkyl, or is absent;
Rf is hydrogen or is absent;
and pharmaceutically acceptable salts, tautomers, isomers, stereoisomers thereof.

4. The compound according to claim 1, wherein:
A is a 6-membered aromatic or heteroaromatic ring;
B is a 6-membered aryl optionally containing one or more N atoms;
Y is selected from the group consisting of hydrogen, halogen, C₁₋₃alkyl, or is absent;
R₁ is hydrogen, methyl, or is selected from the group consisting of: 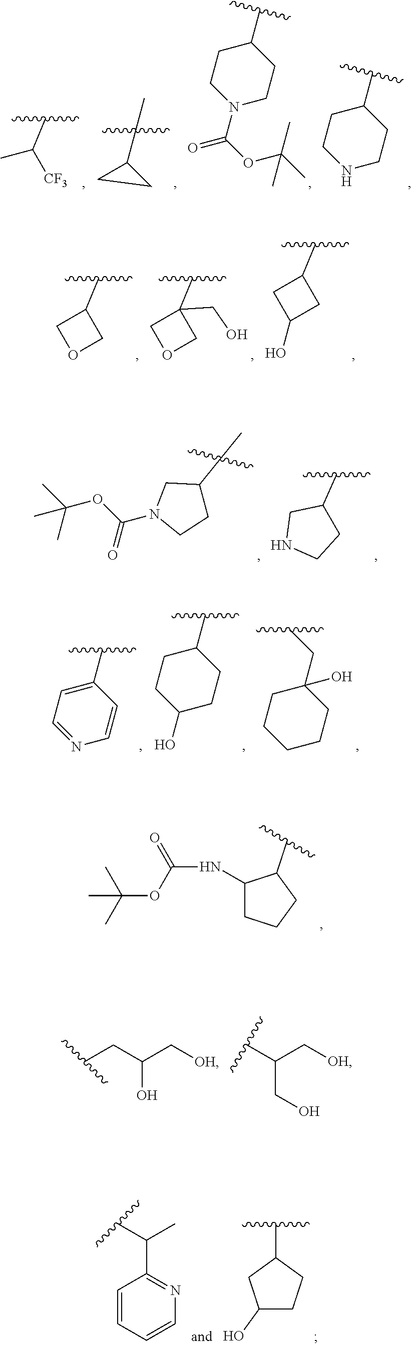
R₂ is H or methyl;
or R₁ and R₂ taken together form with the N atom to which they are attached a heterocyclic ring selected from the group consisting of: 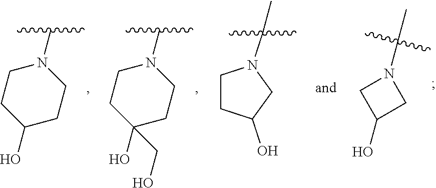 [SMILES: *N1CCC(O)CC1 ; *N1CCC(O)(CO)CC1 ; *N1CCC(O)C1 ; *N1CC(O)C1]
R₃ and R₄ are each independently H or C₁₋₃alkyl; in particular hydrogen or methyl;
Ra is selected from the group consisting of hydrogen, halogen, C₁₋₃alkyl, haloC₁₋₃alkyl and CN; or is absent;
Rb is selected from the group consisting of hydrogen, halogen, C₁₋₃alkyl, haloC₁₋₃alkyl and CN; or is absent;
Rc is selected from the group consisting of hydrogen, halogen, C₁₋₃alkyl, haloC₁₋₃alkyl and CN; or is absent;
Rd is selected from the group consisting of hydrogen, halogen, C₁₋₃alkyl, haloC₁₋₃alkyl and CN; or is absent;
Re is selected from the group consisting of hydrogen, halogen, C₁₋₃alkyl, or is absent;
Rf is hydrogen; or is absent;
and pharmaceutically acceptable salts, tautomers, isomers, stereoisomers thereof.

5. A compound of claim 1, wherein:
A is phenyl or pyridyl;
B is phenyl or pyridyl;
and pharmaceutically acceptable salts, tautomers, isomers, stereoisomers thereof.

6. A compound of claim 1, wherein A is phenyl and B is phenyl and pharmaceutically acceptable salts, tautomers, isomers, stereoisomers thereof.

7. A compound of claim 1, wherein at least one of Ra, Rb, Re and Rd is F and the other(s) is/are hydrogen and pharmaceutically acceptable salts, tautomers, isomers, stereoisomers thereof.

8. A compound according to claim 1 selected from the group consisting of:
4-amino-3-(N-methylsulfamoyl)-N-(3,4,5-trifluorophenyl)benzamide;
4-amino-3-sulfamoyl-N-(3,4,5-trifluorophenyl)benzamide;
4-amino-N-(3,4-difluorophenyl)-3-sulfamoylbenzamide;
4-amino-2-chloro-5-sulfamoyl-N-(3,4,5-trifluorophenyl)benzamide;
4-amino-2-bromo-5-sulfamoyl-N-(3,4,5-trifluorophenyl)benzamide;
4-amino-N-(4-fluoro-3-(trifluoromethyl)phenyl)-3-sulfamoylbenzamide;
4-amino-N-(3-cyano-4-fluorophenyl)-3-sulfamoylbenzamide;
4-amino-N-(3-(difluoromethyl)-4-fluorophenyl)-3-sulfamoylbenzamide;
4-amino-N-(3-chloro-4-fluorophenyl)-3-sulfamoylbenzamide;
4-amino-N-(4-fluoro-3-methylphenyl)-3-sulfamoylbenzamide;
4-amino-2-methyl-5-sulfamoyl-N-(3,4,5-trifluorophenyl)benzamide;
(R)-4-amino-N-(3,4,5-trifluorophenyl)-3-(N-(1,1,1-trifluoropropan-2-yl)sulfamoyl)benzamide;
(S)-4-amino-N-(3,4,5-trifluorophenyl)-3-(N-(1,1,1-trifluoropropan-2-yl)sulfamoyl)benzamide;
4-amino-3-(N-cyclopropylsulfamoyl)-N-(3,4,5-trifluorophenyl)benzamide;
trans-4-amino-3-(N-(4-hydroxycyclohexyl)sulfamoyl)-N-(3,4,5-trifluorophenyl)benzamide;
cis-4-amino-3-(N-(4-hydroxycyclohexyl)sulfamoyl)-N-(3,4,5-trifluorophenyl)benzamide;
trans-4-amino-5-(N-(4-hydroxycyclohexyl)sulfamoyl)-2-methyl-N-(3,4,5-trifluorophenyl) benzamide;
cis-4-amino-3-(N-3-hydroxycyclobutyl)sulfamoyl)-N-(3,4,5-trifluorophenyl)benzamide;
trans-4-amino-3-(N-3-hydroxycyclobutyl)sulfamoyl)-N-(3,4,5-trifluorophenyl)benzamide;
4-amino-3-(N-((1R,3R)-3-hydroxycyclopentyl)sulfamoyl)-N-(3,4,5-trifluorophenyl)benzamide;
4-amino-3-((4-hydroxypiperidin-1-yl)sulfonyl)-N-(3,4,5-trifluorophenyl)benzamide;
4-amino-3-(N-(oxetan-3-yl)sulfamoyl)-N-(3,4,5-trifluorophenyl)benzamide;
tert-butyl(S)-3-((2-amino-5-((3,4,5-trifluorophenyl)carbamoyl)phenyl)sulfonamido) pyrrolidine-1-carboxylate;
4-amino-3-methyl-5-sulfamoyl-N-(3,4,5-trifluorophenyl)benzamide;
4-amino-3-(N-(3-(hydroxymethyl)oxetan-3-yl)sulfamoyl)-N-(3,4,5-trifluorophenyl)benzamide;
4-amino-3-(N-((1-hydroxycyclohexyl)methyl)sulfamoyl)-N-(3,4,5-trifluorophenyl)benzamide;
4-amino-N-(4-fluoro-3-methylphenyl)-2-methyl-5-sulfamoylbenzamide;
4-amino-5-(N-((1R,4R)-4-hydroxycyclohexyl)sulfamoyl)-2-methyl-N-(3,4,5-trifluorophenyl) benzamide;
trans-4-amino-N-(3-chloro-4-fluorophenyl)-3-(N-(4-hydroxycyclohexyl)sulfamoyl)benzamide;
4-amino-N-(3-(difluoromethyl)-4-fluorophenyl)-3-(N-((1r1R,4r4R)-4-hydroxycyclohexyl) sulfamoyl)benzamide;
trans-4-amino-N-(3-(difluoromethyl)-4-fluorophenyl)-3-(N-(4-hydroxycyclohexyl)sulfamoyl) benzamide;
4-amino-3-(N-((1S,3R)-3-hydroxycyclopentyl)sulfamoyl)-N-(3,4,5-trifluorophenyl)benzamide;
4-amino-3-(N-((1R,3S)-3-hydroxycyclopentyl)sulfamoyl)-N-(3,4,5-trifluorophenyl)benzamide;
4-amino-3-((4-hydroxy-4-(hydroxymethyl)piperidin-1-yl)sulfonyl)-N-(3,4,5-trifluorophenyl)benzamide;
tert-butyl((1R,2S)-2-((2-amino-5-((3,4,5-trifluorophenyl)carbamoyl)phenyl)sulfonamido) cyclopentyl)carbamate;
tert-butyl((1S,2R)-2-((2-amino-5-((3,4,5-trifluorophenyl)carbamoyl)phenyl)sulfonamido) cyclopentyl)carbamate;
4-amino-3-((3-hydroxypyrrolidin-1-yl)sulfonyl)-N-(3,4,5-trifluorophenyl)benzamide;
4-amino-N-(3-chloro-4-fluorophenyl)-3-((4-hydroxypiperidin-1-yl)sulfonyl)benzamide;
4-amino-N-(3-chloro-4-fluorophenyl)-3-((3-hydroxyazetidin-1-yl)sulfonyl)benzamide;
4-amino-3-(N-(2,3-dihydroxypropyl)sulfamoyl)-N-(3,4,5-trifluorophenyl)benzamide;
trans-4-amino-3-(N-(3-hydroxycyclopentyl)sulfamoyl)-N-(3,4,5-trifluorophenyl)benzamide;
trans-2-amino-5-(N-(4-hydroxycyclohexyl)sulfamoyl)-N-(3,4,5-trifluorophenyl)benzamide;
2-amino-5-((4-hydroxypiperidin-1-yl)sulfonyl)-N-(3,4,5-trifluorophenyl)benzamide;
(R)-4-amino-2-methyl-N-(3,4,5-trifluorophenyl)-5-(N-(1,1,1-trifluoropropan-2-yl)sulfamoyl) benzamide;
(S)-4-amino-2-methyl-N-(3,4,5-trifluorophenyl)-5-(N-(1,1,1-trifluoropropan-2-yl)sulfamoyl) benzamide;
4-amino-N-(3-chloro-4,5-difluorophenyl)-2-methyl-5-sulfamoylbenzamide;
4-amino-N-(4-fluoro-3-(trifluoromethyl)phenyl)-2-methyl-5-sulfamoylbenzamide;
4-amino-N-(3-(difluoromethyl)-4-fluorophenyl)-2-methyl-5-sulfamoylbenzamide;
4-amino-N-(3-cyano-4-fluorophenyl)-2-methyl-5-sulfamoylbenzamide;
4-amino-N-(3-chloro-4-fluorophenyl)-2-methyl-5-sulfamoylbenzamide;
and pharmaceutically acceptable salts, tautomers, isomers, stereoisomers thereof.

9. (canceled)

10. A method for the treatment and/or prevention of an HBV infection and/or a condition related to an HBV infection, comprising administering compound or a pharmaceutically acceptable salt, tautomer, isomer or stereoisomer thereof of claim 1 to a patient in need thereof.

11. A method for the treatment and/or prevention of an HBV infection and/or a condition related to an HBV infection, comprising administering a compound of general formula (I): 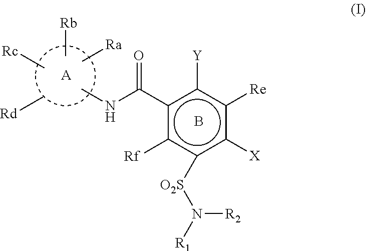
wherein:
A is a 6-membered aromatic or heteroaromatic ring;
B is a 6-membered aryl optionally containing one or more N atoms;
X is H or NR₃R₄;
Y is selected from the group consisting of hydrogen, halogen, C₁₋₆alkyl, NH₂, NH(C₁₋₆alkyl), N(CH₃)₂, NHC(O)CH₃, OH, saturated or partially unsaturated C₃₋₇cycloalkyl, 5- or 6-membered heteroaryl and CN or is absent;
with the proviso that, when X is H, Y is selected form the group consisting of NH₂, NH(C₁₋₆alkyl), N(CH₃)₂, NHC(O)CH₃;
R₁ and R₂ are each independently selected from H, linear or branched C₁₋₆alkyl, saturated or partially unsaturated
C₃₋₇cycloalkyl, C₃₋₇heterocycloalkyl and heteroaryl, each of said linear or branched C₁₋₆alkyl, saturated or partially unsaturated C₃₋₇cycloalkyl, C₃₋₇heterocycloalkyl or heteroaryl group being optionally substituted with one or more substituents selected from OH, halogen, NH₂, NH(C═O)OC₁₋₆alkyl, NH(C₁₋₆alkyl), C₁₋₆alkyl, C₃₋₇cycloalkyl, C₃₋₇heterocycloalkyl, C₁₋₆hydroxyalkyl, 5- or 6-membered heteroaryl, C(═O)C₁₋₆alkyl, C(═O)OC₁₋₆alkyl, OC₁₋₆alkyl, O(CH₂)ₙC₃₋₁₀cycloalkyl and O(CH₂)ₙC₃₋₁₀heterocycloalkyl;
or R₁ and R₂ taken together form with the N atom to which they are attached a saturated or partially unsaturated 3-10 membered heterocyclic ring optionally containing another heteroatom selected from N, O and S, said saturated or partially unsaturated 3-10 membered heterocyclic ring being optionally substituted with one or more substituents selected from OH, halogen, C₁₋₆alkyl, C₁₋₆haloalkyl and (CH₂)ₙR₅;
each occurrence of n is independently 0, 1, 2, 3 or 4;
R₃ and R₄ are each independently H, or linear or branched C₁₋₃alkyl optionally substituted with one or more groups selected from halogen, NH₂, NHC₁₋₆alkyl, N(C₁₋₆alkyl)₂, NH(C═O)C₁₋₆alkyl, NH(C═O)OC₁₋₆alkyl, OC₁₋₆alkyl, O(CH₂)ₙC₃₋₁₀cycloalkyl and O(CH₂)ₙC₃₋₁₀heterocycloalkyl, with the proviso that NR₃R₄ does not form a saturated, partially saturated or unsaturated heterocyclic ring;
R₅ is selected from the group consisting of OH, NH₂, NH(CH₃), N(CH₃)₂, NHC(O)CH₃, CN, haloC₁₋₃alkyl, C₁₋₃alkoxy, haloC₁₋₃alkoxy, heterocyclic ring, aryl and heteroaryl;
Ra is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O) CH₃, OH and CN; or is absent;
Rb is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rc is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Rd is selected from the group consisting of hydrogen, halogen, C₁₋₃alkyl, haloC₁₋₃alkyl, C₁₋₃alkoxy, haloC₁₋₃alkoxy, NH₂, NH(CH₃), N(CH₃)₂, NHC(O)CH₃, OH and CN; or is absent;
Re is selected from the group consisting of hydrogen, halogen, C₁₋₃alkyl; or is absent;
Rf is hydrogen, halogen, C₁₋₃alkyl; or is absent;
and pharmaceutically acceptable salts, tautomers, isomers, stereoisomers thereof, to a patient in need thereof.

12. The method according to claim 10, wherein said condition related to an HBV infection is selected from the group consisting of: chronic hepatitis B, HBV/HDV co-infection, HBV/HCV co-infection, HBV/HIV co-infection, inflammation, necrosis, cirrhosis, hepatocellular carcinoma, hepatic decompensation and hepatic injury from an HBV infection.

13. The method according to claim 10, wherein said use is in treating, eradicating, reducing, slowing or inhibiting an HBV infection in an individual in need thereof, and/or in reducing the viral load associated with an HBV infection in an individual in need thereof, and/or in reducing reoccurrence of an HBV infection in an individual in need thereof, and/or in inducing remission of hepatic injury from an HBV infection in an individual in need thereof, and/or in prophylactically treating an HBV infection in an individual afflicted with a latent HBV infection.

14. The method according to claim 10, wherein said treatment is in combination with at least one further therapeutic agent.

15. The method according to claim 14, wherein the at least one further therapeutic agent is selected from the group consisting of: a therapeutic vaccine; an RNA interference therapeutic/antisense oligonucleotide; an immunomodulator; a STING agonist; a RIG-I modulator; a NKT modulator; an IL agonist; an interleukin or another immune acting protein; a therapeutic and prophylactic vaccine; an immune checkpoint modulator/inhibitor; an HBV entry inhibitor; a cccDNA modulator; an inhibitor of HBV protein espression; an agent targeting HBV RNA; a capsid assembly inhibitor/modulator; a core or X protein targeting agent; a nucleotide analogue; a nucleoside analogue; an interferon or a modified interferon; an HBV antiviral of distinct or unknown mechanism; a cyclophilin inhibitor; a sAg release inhibitor; an HBV polymerase inhibitor; a dinucleotide; a SMAC inhibitor; a HDV targeting agent; a viral maturation inhibitor; a reverse transcriptase inhibitor and an HBV RNA destabilizer or another small-molecule inhibitor of HBV protein expression; or a combination thereof.

16. The method according to claim 15, wherein said therapeutic vaccine is selected from: HBsAG-HBIG, HB-Vac, ABX203, NASVAC, GS-4774, GX-110 (HB-110E), CVI-HBV-002, RG7944 (INO-1800), TG-1050, FP-02 (Hepsyn-B), AIC649, VGX-6200, KW-2, TomegaVax-HBV, ISA-204, NU-500, INX-102-00557, HBV MVA and PepTcell; wherein said RNA interference therapeutic is selected from: TKM-HBV (ARB-1467), ARB-1740, ARC-520, ARC-521, BB-HB-331, REP-2139, ALN-HBV, ALN-PDL, LUNAR-HBV, GS3228836 and GS3389404; wherein said immunomodulator is a TLR agonist; wherein said RIG-I modulator is SB-9200; wherein said IL agonist or other immune acting protein is INO-9112 or recombinant IL12; wherein said immune checkpoint modulator/inhibitor is BMS-936558 (Opdivo (nivolumab)) or pembrolizumab; wherein said HBV entry inhibitor is Myrcludex B, IVIG-Tonrol or GC-1102; wherein said cccDNA modulator is selected from: a direct cccDNA inhibitor, an inhibitor of cccDNA formation or maintenance, a cccDNA epigenetic modifier and an inhibitor of cccDNA transcription; wherein said capsid assembly inhibitor/modulator, core or X protein targeting agent, direct cccDNA inhibitor, inhibitor of cccDNA formation or maintenance, or cccDNA epigenetic modifier is selected from: BAY 41-4109, NVR 3-778, GLS-4, NZ-4 (W28F), Y101, ARB-423, ARB-199, ARB-596, AB-506, JNJ-56136379, ASMB-101 (AB-V102), ASMB-103, CHR-101, CC-31326, AT-130 and R07049389; wherein said interferon or modified interferon is selected from: interferon alpha (IFN-α), pegylated interferon alpha (PEG-IFN-α), interferon alpha-2a, recombinant interferon alpha-2a, peginterferon alpha-2a (Pegasys), interferon alpha-2b (Intron A), recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-λ), peginterferon lambda-1, interferon omega, interferon tau, interferon gamma (IFN-γ), interferon alfacon-1, interferon alpha-n1, interferon alpha-n3, albinterferon alpha-2b, BLX-883, DA-3021, PI 101 (also known as AOP2014), PEG-infergen, Belerofon, INTEFEN-IFN, albumin/interferon alpha 2a fusion protein, rHSA-IFN alpha 2a, rHSA-IFN alpha 2b, PEG-IFN-SA and interferon alpha biobetter; wherein said HBV antiviral of distinct or unknown mechanism is selected from: AT-61 ((E)-N-(1-chloro-3-oxo-1-phenyl-3-(piperidin-1-yl)prop-1-en-2-yl)benzamide), AT130 ((E)-N-(1-bromo-1-(2-methoxyphenyl)-3-oxo-3-(piperidin-1-yl)prop-1-en-2-yl)-4-nitrobenzamide), analogues thereof, REP-9AC (REP-2055), REP-9AC′ (REP-2139), REP-2165 and HBV-0259; wherein said cyclophilin inhibitor is selected from: OCB-030 (NVP-018), SCY-635, SCY-575 and CPI-431-32; wherein said HBV polymerase inhibitor is selected from: entecavir (Baraclude, Entavir), lamivudine (3TC, Zeffix, Heptovir, Epivir, and Epivir-HBV), telbivudine (Tyzeka, Sebivo), clevudine, besifovir, adefovir (hepsera), tenofovir, tenofovir disoproxil fumarate (Viread), tenofovir alafenamide fumarate (TAF), tenofovir disoproxil orotate (DA-2802), tenofovir disopropxil aspartate (CKD-390), AGX-1009, and CMX157; wherein said dinucleotide is SB9200; wherein said SMAC inhibitor is Birinapant; wherein said HDV targeting agent is Lonafamib; wherein said HBV RNA destabilizer or other small-molecule inhibitor of HBV protein expression is RG7834 or AB-452.

17. A pharmaceutical composition comprising the compound or the pharmaceutically acceptable salt, tautomer, isomer, stereoisomer thereof as defined in claim 1, alone or in combination with at least one further therapeutic agent, and at least one pharmaceutically acceptable excipient.

18. The pharmaceutical composition according to claim 17, wherein the at least one further therapeutic agent is selected from the group consisting of: a therapeutic vaccine; an RNA interference therapeutic/antisense oligonucleotide; an immunomodulator; a STING agonist; a RIG-I modulator; a NKT modulator; an IL agonist; an interleukin or another immune acting protein; a therapeutic and prophylactic vaccine; an immune checkpoint modulator/inhibitor; an HBV entry inhibitor; a cccDNA modulator; an inhibitor of HBV protein espression; an agent targeting HBV RNA; a capsid assembly inhibitor/modulator; a core or X protein targeting agent; a nucleotide analogue; a nucleoside analogue; an interferon or a modified interferon; an HBV antiviral of distinct or unknown mechanism; a cyclophilin inhibitor; a sAg release inhibitor; an HBV polymerase inhibitor; a dinucleotide; a SMAC inhibitor; a HDV targeting agent; a viral maturation inhibitor; a reverse transcriptase inhibitor and an HBV RNA destabilizer or another small-molecule inhibitor of HBV protein expression; or a combination thereof.

19. The pharmaceutical composition according to claim 18, wherein said therapeutic vaccine is selected from: HBsAG-HBIG, HB-Vac, ABX203, NASVAC, GS-4774, GX-110 (HB-110E), CVI-HBV-002, RG7944 (INO-1800), TG-1050, FP-02 (Hepsyn-B), AIC649, VGX-6200, KW-2, TomegaVax-HBV, ISA-204, NU-500, INX-102-00557, HBV MVA and PepTcell; wherein said RNA interference therapeutic is selected from: TKM-HBV (ARB-1467), ARB-1740, ARC-520, ARC-521, BB-HB-331, REP-2139, ALN-HBV, ALN-PDL, LUNAR-HBV, GS3228836 and GS3389404; wherein said immunomodulator is a TLR agonist; wherein said RIG-I modulator is SB-9200; wherein said IL agonist or other immune acting protein is INO-9112 or recombinant IL12; wherein said immune checkpoint modulator/inhibitor is BMS-936558 (Opdivo (nivolumab)) or pembrolizumab; wherein said HBV entry inhibitor is Myrcludex B, IVIG-Tonrol or GC-1102; wherein said cccDNA modulator is selected from: a direct cccDNA inhibitor, an inhibitor of cccDNA formation or maintenance, a cccDNA epigenetic modifier and an inhibitor of cccDNA transcription; wherein said capsid assembly inhibitor/modulator, core or X protein targeting agent, direct cccDNA inhibitor, inhibitor of cccDNA formation or maintenance, or cccDNA epigenetic modifier is selected from: BAY 41-4109, NVR 3-778, GLS-4, NZ-4 (W28F), Y101, ARB-423, ARB-199, ARB-596, AB-506, JNJ-56136379, ASMB-101 (AB-V102), ASMB-103, CHR-101, CC-31326, AT-130 and R07049389; wherein said interferon or modified interferon is selected from: interferon alpha (IFN-α), pegylated interferon alpha (PEG-IFN-α), interferon alpha-2a, recombinant interferon alpha-2a, peginterferon alpha-2a (Pegasys), interferon alpha-2b (Intron A), recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-λ), peginterferon lambda-1, interferon omega, interferon tau, interferon gamma (IFN-γ), interferon alfacon-1, interferon alpha-n1, interferon alpha-n3, albinterferon alpha-2b, BLX-883, DA-3021, PI 101 (also known as AOP2014), PEG-infergen, Belerofon, INTEFEN-IFN, albumin/interferon alpha 2a fusion protein, rHSA-IFN alpha 2a, rHSA-IFN alpha 2b, PEG-IFN-SA and interferon alpha biobetter; wherein said HBV antiviral of distinct or unknown mechanism is selected from: AT-61 ((E)-N-(1-chloro-3-oxo-1-phenyl-3-(piperidin-1-yl)prop-1-en-2-yl)benzamide), AT130 ((E)-N-(1-bromo-1-(2-methoxyphenyl)-3-oxo-3-(piperidin-1-yl)prop-1-en-2-yl)-4-nitrobenzamide), analogues thereof, REP-9AC (REP-2055), REP-9AC′ (REP-2139), REP-2165 and HBV-0259; wherein said cyclophilin inhibitor is selected from: OCB-030 (NVP-018), SCY-635, SCY-575 and CPI-431-32; wherein said HBV polymerase inhibitor is selected from: entecavir (Baraclude, Entavir), lamivudine (3TC, Zeffix, Heptovir, Epivir, and Epivir-HBV), telbivudine (Tyzeka, Sebivo), clevudine, besifovir, adefovir (hepsera), tenofovir, tenofovir disoproxil fumarate (Viread), tenofovir alafenamide fumarate (TAF), tenofovir disoproxil orotate (DA-2802), tenofovir disopropxil aspartate (CKD-390), AGX-1009, and CMX157; wherein said dinucleotide is SB9200; wherein said SMAC inhibitor is Birinapant; wherein said HDV targeting agent is Lonafamib; wherein said HBV RNA destabilizer or other small-molecule inhibitor of HBV protein expression is RG7834 or AB-452.

20-22. (canceled)

23. A process for the synthesis of the compound of formula (I) or the pharmaceutically acceptable salt, tautomer, solvate, isomer or stereoisomer thereof as defined in claim 1, said process comprising at least one of the following steps: 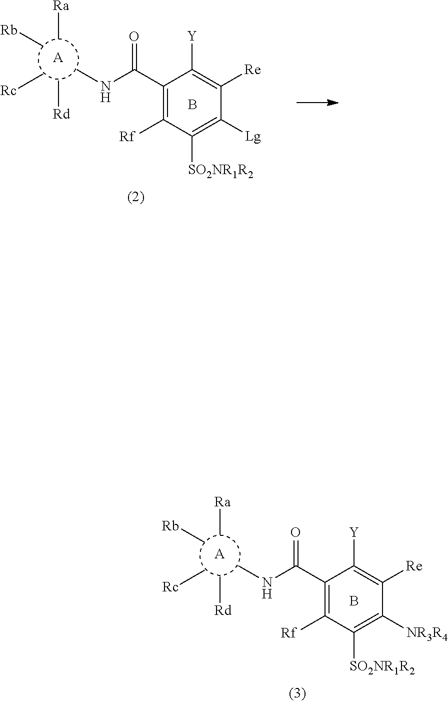
reacting a compound of formula (2) with an amine of formula NHR₃R₄ to obtain a compound of formula (3), wherein A, B, Ra, Rb, Rc, Rd, Re, Rf, Y, R₁, R₂, R₃ and R₄ are as defined in claim 1, and Lg is a leaving group such as Cl or F; or 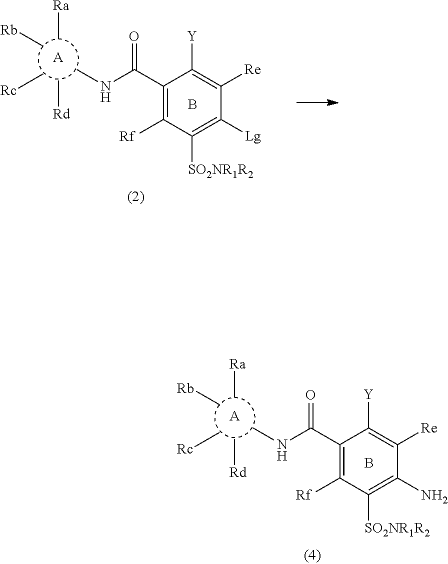
reacting a compound of formula (2) with an ammonium salt such as NH₄OH to obtain a compound of formula (4), wherein A, B, Ra, Rb, Rc, Rd, Re, Rf, Y, R₁ and R₂ are as defined in claim 1, and Lg is a leaving group such as Cl or F; or 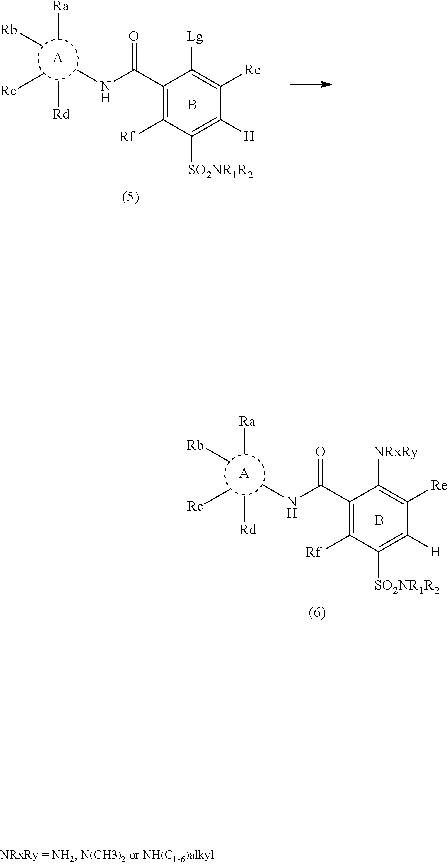
reacting a compound of formula (5) with an amine of formula (CH₃)₂NH or (C₁₋₆)alkylNH₂ or with NH₄OH to obtain a compound of formula (6) wherein A, B, Ra, Rb, Rc, Rd, Re, Rf, R₁ and R₂ are as defined in claim 1 and Lg is a leaving group such as Cl or F.


SEQUENCE LISTING (4 sequences)

SEQ ID NO: 1 - DNA, 22 bp, Artificial Sequence
  synthetic primer
atttgttcagtggttcgtaggg

SEQ ID NO: 2 - DNA, 22 bp, Artificial Sequence
  synthetic primer
cggtaaaaagggactcaagatg

SEQ ID NO: 3 - DNA, 22 bp, Artificial Sequence
  synthetic primer
tgcttaatcagtgaggcaccta

SEQ ID NO: 4 - DNA, 22 bp, Artificial Sequence
  synthetic primer
agccctcccgtatcgtagttat